(19) United States
(12) Patent Application Publication
(10) Pub. No.: US 2023/0314417 A1
(43) Pub. Date: Oct. 5, 2023

(54) MONOCLONAL ANTIBODIES TO CHEMICALLY-MODIFIED NUCLEIC ACIDS AND USES THEREOF

(71) Applicant: AMGEN INC., Thousand Oaks, CA (US)

(72) Inventors: Brian CHAN, Port Coquitlam (CA); Weihsu Claire CHEN, Vancouver (CA); Sara HUMPHREYS, Oakland, CA (US); Agnieszka KIELCZEWSKA, Vancouver (CA); Mai THAYER, Alameda, CA (US)

(73) Assignee: AMGEN INC., Thousand Oaks, CA (US)

(21) Appl. No.: 17/913,629

(22) PCT Filed: Mar. 22, 2021
(86) PCT No.: PCT/US2021/023538

§ 371 Date: Sep. 22, 2022

Related U.S. Application Data
(60) Provisional application No. 62/993,575, filed on Mar. 23, 2020.

(51) Int. Cl. G01N 33/53; C07K 16/44; G01N 33/531; C12N 15/113

(52) U.S. Cl. CPC G01N 33/5308; C07K 16/44; G01N 33/531; C12N 15/113; C12N 2310/14; C12N 2310/351; C12N 2310/321; C12N 2310/313

(57) ABSTRACT

The present invention relates to monoclonal antibodies that specifically bind to chemically-modified nucleic acid molecules, including pan-specific antibodies that bind to chemically-modified nucleic acid molecules independent of nucleotide sequence. The invention also relates to methods of generating monoclonal antibodies to chemically-modified nucleic acid molecules as well as methods of using such antibodies to detect nucleic acid molecules in biological samples. Various immunoassays incorporating the monoclonal antibodies of the invention are also disclosed.

### CROSS-REFERENCE TO RELATED APPLICATIONS

This application claims the benefit of U.S. Provisional Application No. 62/993,575, filed Mar. 23, 2020, which is hereby incorporated by reference in its entirety.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Mar. 22, 2021, is named A-2544-WO-PCT ST25 and is 176 kilobytes in size.

### FIELD OF THE INVENTION

The present invention relates to the fields of bioanalytics and immunology. In particular, the present invention relates to monoclonal antibodies that specifically bind to chemically-modified nucleic acid molecules and methods for generating such monoclonal antibodies. The present invention also relates to methods of using the monoclonal antibodies, for example, to detect nucleic acid molecules in biological samples, including samples from subjects treated with chemically-modified nucleic molecules.

### BACKGROUND OF THE INVENTION

Nucleic acid-based drugs have continued to develop as a unique and effective therapeutic class with numerous nucleic acid therapies being studied in clinical trials and others receiving regulatory approval for various indications in humans (Sridharan and Gogtay, British Journal of Clinical Pharmacology, Vol. 82: 659-672, 2016; Stein et al., Molecular Therapy, Vol. 25: 1069-1075, 2017; Adams et al., New England Journal of Medicine, Vol. 379: 11-21, 2018). The development of therapeutic nucleic acid molecules requires an understanding of the pharmacokinetic parameters, metabolism and distribution of the molecules. Thus, robust and sensitive bioanalytical assays and reagents capable of detecting nucleic acid molecules in bodily fluids, cells, tissues, and tissue samples are essential. Although various polymerase-chain reaction (Cheng et al., Oligonucleotides, Vol. 19: 203-208, 2009; Cheng et al., In: Therapeutic Oligonucleotides: Methods and Protocols (Goodchild, ed.), Humana Press, pgs. 183-197, 2011), size-exclusion chromatography (Shimoyama et al., Journal of Pharmaceutical and Biomedical Analysis, Vol. 136: 55-65, 2017), and liquid chromatography-mass spectrometry-based methods (Basin et al., Bioanalysis, Vol. 6: 1525-1542, 2014; Ewles et al., Bioanalysis, Vol. 6: 447-464, 2014) exist, they are limited by their sensitivities and time-consuming extraction steps.

Immunoassays offer high specificity, high-throughput, and high sensitivity for the analysis of a broad range of analytes in biological samples (Darwish, Int. J. Biomed. Sci., Vol. 2: 217-235, 2006). However, effective immunoassays require immunoanalytical reagents, namely antibodies that specifically bind to the analyte of interest. The generation of antibodies to nucleic acids is challenging as nucleic acids are poorly immunogenic (see e.g., Hu et al., Expert Rev. Mol. Diagn., Vol. 14: 895-916, 2014 and Feederle and Schepers, RNA Biology, Vol. 14: 1089-1098, 2017). Even more difficult is the production of antibodies, particularly monoclonal antibodies, that recognize chemically-modified nucleic acid molecules independent of nucleotide sequence or pattern of chemical modifications because most antibodies that recognize modified nucleotides are directed to single modified nucleotides (Feederle and Schepers, 2017).

Thus, there is a need in the art for novel antibodies that specifically bind to chemically-modified nucleic acid molecules and methods of generating such antibodies. Such antibodies are particularly useful for the development of novel immunoassays to detect chemically-modified nucleic acid molecules in various types of biological samples.

### SUMMARY OF THE INVENTION

The present invention provides monoclonal antibodies that specifically bind to chemically-modified nucleic acid molecules as well as methods of generating such antibodies. The present invention also provides methods of using such antibodies to detect chemically-modified nucleic acid molecules in biological samples, particularly samples obtained from subjects administered the chemically-modified nucleic acid molecules.

In some embodiments, the present invention provides a method for generating a monoclonal antibody that specifically binds to a chemically-modified nucleic acid molecule. In one embodiment, the method comprises conjugating a plurality of nucleic acid molecules to a bead to form an immunogen, wherein the nucleic acid molecules each comprise one or more modified nucleotides; administering the immunogen to an animal; obtaining splenocytes from the immunized animal; selecting splenocytes that are IgG positive and bind to the chemically-modified nucleic acid molecule thereby isolating antigen-specific antibody producing cells; plating the antigen-specific antibody producing cells in single-cell culture; and isolating the monoclonal antibody from the single-cell culture. In certain embodiments, the methods further comprise lysing the B-cells derived from the single-cell culture and sequencing the antibody genes from the clonal B cells. In alternative embodiments, the methods comprise conjugating a plurality of nucleic acid molecules to a bead to form an immunogen, wherein the nucleic acid molecules each comprise one or more modified nucleotides; administering the immunogen to an animal; obtaining splenocytes from the immunized animal; fusing the splenocytes to a myeloma cell line, thereby generating hybridoma cells; establishing hybridoma cell lines from the hybridoma cells, and identifying a hybridoma cell line that produces an antibody that binds to the chemically-modified nucleic acid molecules of interest. The animal to be administered the immunogen can be any immunocompetent mammal, including a mouse, rabbit, rat, goat, or non-human primate. In one particular embodiment, the animal to be administered the immunogen is a rabbit.

The present invention includes monoclonal antibodies or antigen-binding fragments thereof produced by any of the methods described herein. The monoclonal antibodies and antigen-binding fragments thereof find use in a variety of applications, including detection and isolation of chemically-modified nucleic acid molecules in biological fluids and tissues, for example, using immunoassay, immunoprecipitation, and immunohistochemistry techniques. In some embodiments, the present invention provides antibodies that specifically bind to chemically-modified nucleic acid molecules independent of nucleotide sequence. Antibodies with such a binding specificity are referred to herein as pan-specific antibodies. In certain embodiments, the pan-specific antibodies or antigen-binding fragments thereof of the invention comprise one or more CDRs or variable regions from any of the pan-specific antibodies described herein. For instance, in some embodiments, the pan-specific antibodies or antigen-binding fragments thereof comprise a CDRL1 comprising a sequence selected from SEQ ID NOs: 1-3; a CDRL2 comprising the sequence of SEQ ID NOs: 14-16; a CDRL3 comprising a sequence selected from SEQ ID NOs: 25-27; a CDRH1 comprising a sequence selected from SEQ ID NOs: 51-53; a CDRH2 comprising a sequence selected from SEQ ID NOs: 64-66; and a CDRH3 comprising a sequence selected from SEQ ID NOs: 77-79. The pan-specific antibodies or antigen-binding fragments thereof may comprise a light chain variable region that comprises a sequence that is at least 90% identical or at least 95% identical to a sequence selected from SEQ ID NOs: 38-40. In these and other embodiments, the pan-specific antibodies or antigen-binding fragments thereof of the invention comprise a heavy chain variable region that comprises a sequence that is at least 90% identical or at least 95% identical to a sequence selected from SEQ ID NOs: 90-92. In one embodiment, the pan-specific antibodies or antigen-binding fragments thereof comprise a light chain variable region comprising a sequence selected from SEQ ID NOs: 38-40 and a heavy chain variable region comprising a sequence selected from SEQ ID NOs: 90-92.

In other embodiments, the present invention provides antibodies that bind in a sequence specific manner to an RNAi construct comprising the nucleotide sequence of SEQ ID NO: 192. Such antibodies, which are referred to herein as 1851 RNAi construct-specific antibodies, specifically bind to the 1851 RNAi construct molecule described herein and do not significantly bind or cross-react with other chemically-modified nucleic acid molecules having different nucleotide sequences. The 1851 RNAi construct-specific antibodies or antigen-binding fragments thereof of the invention may comprise one or more CDRs or variable regions from any of the 1851 RNAi construct-specific antibodies described herein. For instance, in some embodiments, the 1851 RNAi construct-specific antibodies or antigen-binding fragments thereof comprise a CDRL1 comprising a sequence selected from SEQ ID NOs: 4-8; a CDRL2 comprising the sequence of SEQ ID NOs: 17-20; a CDRL3 comprising a sequence selected from SEQ ID NOs: 28-32; a CDRH1 comprising a sequence selected from SEQ ID NOs: 54-58; a CDRH2 comprising a sequence selected from SEQ ID NOs: 67-71; and a CDRH3 comprising a sequence selected from SEQ ID NOs: 80-84. The 1851 RNAi construct-specific antibodies or antigen-binding fragments thereof may comprise a light chain variable region that comprises a sequence that is at least 90% identical or at least 95% identical to a sequence selected from SEQ ID NOs: 41-45. In these and other embodiments, the 1851 RNAi construct-specific antibodies or antigen-binding fragments thereof of the invention comprise a heavy chain variable region that comprises a sequence that is at least 90% identical or at least 95% identical to a sequence selected from SEQ ID NOs: 93-97. In one embodiment, the 1851 RNAi construct-specific antibodies or antigen-binding fragments thereof comprise a light chain variable region comprising a sequence selected from SEQ ID NOs: 41-45 and a heavy chain variable region comprising a sequence selected from SEQ ID NOs: 93-97.

In still other embodiments, the present invention provides antibodies that specifically bind to a N-acetyl-galactosamine (GalNAc) moiety, such as those described herein. These GalNAc moiety-specific antibodies or antigen-binding fragments thereof of the invention may comprise one or more CDRs or variable regions from any of the GalNAc moiety-specific antibodies described herein. For instance, in some embodiments, the GalNAc moiety-specific antibodies or antigen-binding fragments thereof comprise a CDRL1 comprising a sequence selected from SEQ ID NOs: 9-13; a CDRL2 comprising the sequence of SEQ ID NOs: 19 and 21-24; a CDRL3 comprising a sequence selected from SEQ ID NOs: 33-37; a CDRH1 comprising a sequence selected from SEQ ID NOs: 59-63; a CDRH2 comprising a sequence selected from SEQ ID NOs: 72-76; and a CDRH3 comprising a sequence selected from SEQ ID NOs: 85-89. In certain embodiments, the GalNAc moiety-specific antibodies or antigen-binding fragments thereof may comprise a light chain variable region that comprises a sequence that is at least 90% identical or at least 95% identical to a sequence selected from SEQ ID NOs: 46-50. In these and other embodiments, the GalNAc moiety-specific antibodies or antigen-binding fragments thereof of the invention comprise a heavy chain variable region that comprises a sequence that is at least 90% identical or at least 95% identical to a sequence selected from SEQ ID NOs: 98-102. In one embodiment, the GalNAc moiety-specific antibodies or antigen-binding fragments thereof comprise a light chain variable region comprising a sequence selected from SEQ ID NOs: 46-50 and a heavy chain variable region comprising a sequence selected from SEQ ID NOs: 98-102.

The present invention also includes one or more isolated polynucleotides and expression vectors encoding any of the antibodies or antigen-binding fragments described herein or components thereof, as well as recombinant host cells comprising the encoding polynucleotides and expression vectors. Methods of producing the antibodies or antigen-binding fragments of the invention using recombinant methods are also contemplated. In some embodiments, such methods comprise culturing a host cell comprising an expression vector encoding the antibody or antigen-binding fragment under conditions that allow expression of the antibody or antigen-binding fragment, and recovering the antibody or antigen-binding fragment from the culture medium or host cell.

Any of the monoclonal antibodies or antigen-binding fragments thereof described herein can be coupled to a detectable label for use in various immunoassays, including the immunoassays described herein. In some embodiments, the detectable label is a fluorophore (e.g. fluorescein, rhodamine, Alexa dyes molecules, etc.), metallic nanoparticle (e.g. gold nanoparticles, silver nanoparticles, composite nanoparticles, etc.), enzyme (e.g. alkaline phosphatase, horseradish peroxidase, beta-galactosidase, etc.), radiolabel (¹²⁵I, ¹³¹I, ³H, ³⁵S, etc.), or ECL luminophore (e.g. ruthenium complexes, iridium complexes, etc.). In certain embodiments, the detectable label coupled to a monoclonal antibody or antigen-binding fragment of the invention is an ECL luminophore, such as a ruthenium complex.

The present invention provides methods for detecting a chemically-modified nucleic acid molecule in a sample using the monoclonal antibodies or antigen-binding fragments of the invention. In some embodiments, the methods comprise providing a surface comprising a capture antibody that specifically binds to the chemically-modified nucleic acid molecule; contacting the surface with the sample under conditions allowing the chemically-modified nucleic acid molecule, if present in the sample, to bind to the capture antibody on the surface; contacting the surface with a detection reagent, wherein the detection reagent comprises a detectable label coupled to a binding partner that specifically binds to the chemically-modified nucleic acid molecule; and detecting a signal from the detectable label. In certain embodiments, the capture antibody is any of the pan-specific antibodies described herein, such as the 14K10 antibody.

In some embodiments of the detection methods of the invention, the binding partner in the detection reagent can be a labeled form of one of the pan-specific antibodies described herein (e.g. labeled 14K10 antibody) or it can be another antibody that specifically binds to the chemically-modified nucleic acid molecule, such as a polyclonal antibody. The binding partner in the detection reagent can vary depending on the specific chemically-modified nucleic acid molecule to be detected. For instance, in some embodiments in which the chemically-modified nucleic acid molecule to be detected is the 1851 RNAi construct, the binding partner in the detection reagent can be any of the 1851 RNAi construct-specific antibodies described herein. In other embodiments in which the chemically-modified nucleic acid molecule to be detected is covalently linked to a ligand comprising a GalNAc moiety, the binding partner in the detection reagent can be any of the GalNAc moiety-specific antibodies described herein, such as the 14D4 antibody. In still other embodiments in which the chemically-modified nucleic acid molecule to be detected is conjugated to an antibody, the binding partner in the detection reagent can be a target antigen of the antibody, an anti-Fc region antibody, or an anti-idiotypic antibody. The detectable label coupled to the binding partner can be any type of signal-generating entity, such as a fluorophore, metallic nanoparticle, enzyme, radiolabel, or ECL luminophore, such as those described herein.

The present invention also includes methods for detecting an anti-drug antibody to a chemically-modified nucleic acid molecule in a subject using labeled forms of the monoclonal antibodies or antigen-binding fragments thereof of the invention (e.g. pan-specific antibodies) in a competitive immunoassay format. In some embodiments, the method comprises providing a surface comprising the chemically-modified nucleic acid molecule; contacting the surface with a sample obtained from a subject administered the chemically-modified nucleic acid molecule; contacting the surface with a detection reagent, wherein the detection reagent comprises a monoclonal antibody of the invention coupled to a detectable label; and detecting a signal from the detectable label, wherein a signal from the detectable label is indicative of the absence of anti-drug antibody in the sample. In certain embodiments, the detection reagent comprises any one of the pan-specific antibodies of the invention coupled to a detectable label. In one particular embodiment, the detection reagent comprises the 14K10 antibody coupled to a detectable label.

The methods of the invention can be used to generate monoclonal antibodies to or detect or measure in a sample any type of chemically-modified nucleic acid molecule, such as those described herein. In some embodiments, the chemically-modified nucleic acid molecule comprises one or more modified nucleotides selected from 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNAs), or combinations thereof. The chemically-modified nucleic acid molecules may also comprise one or more phosphorothioate internucleotide linkages. In certain embodiments, the chemically-modified nucleic acid molecule used in or detected or measured according to the methods of the invention is double-stranded. For instance, in some embodiments, the chemically-modified nucleic acid molecule is an RNAi construct comprising a sense strand and an antisense strand. In particular embodiments, the chemically-modified nucleic acid molecules to be used in or detected or measured according to the methods of the invention are covalently linked to a ligand, such as any of the ligands described herein. In some embodiments, the ligand comprises a GalNAc moiety. The GalNAc moiety can be a multivalent GalNAc moiety, such as a trivalent or tetravalent GalNAc moiety. In one embodiment, the GalNAc moiety has the structure of Structure 1. In another embodiment, the GalNAc moiety is the TL01 GalNAc moiety. In yet another embodiment, the GalNAc moiety is the TL02 GalNAc moiety. In still another embodiment, the GalNAc moiety is the TL03 GalNAc moiety.

The methods of the invention can be used to detect or measure chemically-modified nucleic acid molecules in a variety of sample types. In some embodiments, the sample is a biological sample, such as serum, plasma, cell lysate, sub-cellular fraction, or tissue (e.g. tissue homogenate). Such samples may be obtained from animal or human subjects who have been administered the chemically-modified nucleic acid molecules. The sample may be obtained from a subject prior to, during, or after treatment with the chemically-modified nucleic acid molecule. In some embodiments, the samples are obtained from cell cultures (e.g. supernatants, sub-cellular fractions, or lysates) that have been exposed to the chemically-modified nucleic acid molecules.

The present invention also encompasses kits for detecting a chemically-modified nucleic acid molecule according to the methods described herein. The kits may comprise different combinations of the monoclonal antibodies or antigen-binding fragments thereof described herein in unlabeled or labeled forms. In some embodiments, the kits comprise a capture antibody immobilized to a surface (e.g. a well in a microtiter plate), wherein the capture antibody specifically binds to the chemically-modified nucleic acid molecule; a detection reagent comprising a detectable label coupled to a binding partner that specifically binds to the chemically-modified nucleic acid molecule; and instructions for contacting the sample with the immobilized capture antibody and detection reagent, and instructions for detecting a signal from the detectable label. In certain embodiments, the capture antibody and the binding partner in the detection reagent are selected from the pan-specific antibodies described herein. In one embodiment, the capture antibody is the 14K10 antibody and the detection reagent comprises a labeled form of the 14K10 antibody. In certain other embodiments, the capture antibody is one of the pan-specific antibodies described herein and the binding partner in the detection reagent is one of the GalNAc moiety-specific antibodies described herein. In one particular embodiment, the capture antibody is the 14K10 antibody and the detection reagent comprises a labeled form of the 14D4 antibody.

### BRIEF DESCRIPTION OF THE DRAWINGS

FIG. 1 is a schematic showing the generation of a multivalent GalNAc-siRNA-coated nanobead immunogen. siRNA molecules, which were conjugated to a trivalent GalNAc moiety (represented as a solid square) at the 5′ end of the sense strand, were covalently attached to biotin (represented as a solid circle) at the 5′ end of the antisense strand. The biotinylated siRNA molecules were attached to streptavidin nanobeads having an average diameter of 0.1 μm.

FIG. 2A is a plot comparing binding of rabbit monoclonal antibodies to the 1851 RNAi construct with the GalNAc moiety versus binding to the 1851 RNAi construct without the GalNAc moiety as measured by a colorimetric ELISA assay. Optical density (OD) values at 450 nm are shown with higher values indicating a higher level of antibody binding. The black box denotes monoclonal antibodies that exhibit a greater level of binding to the 1851 RNAi construct with the GalNAc moiety than the RNAi construct without the GalNAc moiety, indicating that the antibodies likely bind to the GalNAc moiety of the RNAi construct (GalNAc binders).

FIG. 2B is a plot comparing binding of rabbit monoclonal antibodies to the 1851 RNAi construct with the GalNAc moiety versus binding to the 6189 RNAi construct with the GalNAc moiety as measured by a colorimetric ELISA assay. The 6189 RNAi construct has the same GalNAc moiety, chemical modification pattern, and format as the 1851 RNAi construct, but has different nucleotide sequences in the sense and antisense strands. OD values at 450 nm are shown with higher values indicating a higher level of antibody binding. The top black box denotes monoclonal antibodies that exhibit binding to both RNAi constructs, suggesting that these antibodies either bind to the GalNAc moiety (GalNAc binder) or to the double-strand RNA structure independent of nucleotide sequence (pan-siRNA binder). The bottom black box denotes monoclonal antibodies that exhibit a greater level of binding to the 1851 RNAi construct than to the 6189 RNAi construct, indicating that these antibodies specifically bind to the 1851 RNAi construct (1851 binders).

FIG. 3A shows the binding activity as measured by the geometric mean in flow cytometry using a fluorochrome labeled secondary antibody against recombinant antibodies 5I17, 14P2, 14F4, and 14K10 to the indicated antigens. The recombinant antibodies were evaluated for binding at a concentration of 1 μg/mL.

FIG. 3B shows the binding activity as measured by the geometric mean in flow cytometry using a fluorochrome labeled secondary antibody against recombinant antibodies 14D4, 16I3, 16A22, 17D13, and 18J5 to the indicated antigens. The recombinant antibodies were evaluated for binding at a concentration of 1 μg/mL.

FIG. 3C shows the binding activity as measured by the geometric mean in flow cytometry using a fluorochrome labeled secondary antibody against recombinant antibodies 14K23, 17K13, 17F22, 19F24, 20P19, and 20K24 to the indicated antigens. The recombinant antibodies were evaluated for binding at a concentration of 1 μg/mL.

FIG. 4A is a bar graph showing the percent inhibition of the binding of recombinant antibodies 5117, 14F4, and 14K10 to 1851 RNAi construct-coated beads by each of the indicated RNAi constructs as determined by flow cytometry. Each of the indicated RNAi constructs were preincubated with each antibody at a 55:1 molar ratio (RNAi construct:antibody). A higher percent inhibition indicates that the antibody exhibits a higher degree of binding to the indicated RNAi construct.

FIG. 4B is a bar graph showing the percent inhibition of the binding of recombinant antibodies 17K13, 17F22, 19F24, 20P19, and 20K24 to 1851 RNAi construct-coated beads by each of the indicated RNAi constructs as determined by flow cytometry. Each of the indicated RNAi constructs were preincubated with each antibody at a 55:1 molar ratio (RNAi construct:antibody). A higher percent inhibition indicates that the antibody exhibits a higher degree of binding to the indicated RNAi construct.

FIG. 5A is a schematic illustrating one embodiment of an immunoassay method of the invention. In this embodiment, a pan-siRNA antibody (Ab) of the invention is immobilized to a solid surface (e.g. microtiter plate), optionally through a biotin-streptavidin interaction. A sample containing a GalNAc moiety (black triangle) conjugated to a chemically-modified RNAi construct (GalNAc-siRNA conjugate) is contacted with the immobilized pan-siRNA antibody, which recognizes and binds to the double-stranded RNA component of the GalNAc-siRNA conjugate. Detection and quantification of the GalNAc-siRNA conjugate is subsequently accomplished using a labeled pan-siRNA antibody of the invention, such as a ruthenium-labeled pan-siRNA antibody. The capture pan-siRNA antibody may be the same antibody used for the detection pan-siRNA antibody or it can be a different pan-siRNA antibody.

FIG. 5B is a schematic illustrating a second embodiment of an immunoassay method of the invention. In this embodiment, a pan-siRNA antibody (Ab) of the invention is immobilized to a solid surface (e.g. microtiter plate), optionally through a biotin-streptavidin interaction. A sample containing a GalNAc moiety (black triangle) conjugated to a chemically-modified RNAi construct (GalNAc-siRNA conjugate) is contacted with the immobilized pan-siRNA antibody, which recognizes and binds to the double-stranded RNA component of the GalNAc-siRNA conjugate. Detection and quantification of the intact GalNAc-siRNA conjugate is subsequently accomplished using a labeled GalNAc antibody (e.g. ruthenium-labeled GalNAc antibody) of the invention, which binds to the GalNAc component of the GalNAc-siRNA conjugate.

FIG. 5C is a schematic illustrating a third embodiment of an immunoassay method of the invention. In this embodiment, a pan-siRNA antibody (Ab) of the invention is immobilized to a solid surface (e.g. microtiter plate), optionally through a biotin-streptavidin interaction. A sample containing antibody-siRNA conjugate molecules is contacted with the immobilized pan-siRNA antibody, which recognizes and binds to the double-stranded RNA component of the antibody-siRNA conjugate molecule. Detection and quantification of the intact antibody-siRNA conjugate molecule is subsequently accomplished using a labeled binding partner that specifically binds to the antibody component of the conjugate molecule, such as a ruthenium-labeled anti-Fc antibody.

FIG. 6 is a line graph of various concentrations of GalNAc-conjugated RNAi constructs (construct nos. 16081, 16082, 16083, and 16084) in human serum plotted versus electro-chemiluminescent signal in arbitrary units (MSD RFU) measured using the total drug assay format depicted in FIG. 5A (“tot”; open symbols) or the intact drug assay format depicted in FIG. 5B (“int”; solid symbols). Limits of detection (LOD) are shown for each assay format.

FIG. 7A is a line graph of various concentrations of GalNAc-conjugated RNAi constructs (construct nos. 1907, 7213, 8172, and 10927) in human serum plotted versus electro-chemiluminescent signal in arbitrary units (MSD RFU) measured using the intact drug assay format depicted in FIG. 5B.

FIG. 7B is a line graph of various concentrations of GalNAc-conjugated RNAi constructs (construct nos. 1907, 7213, 8172, and 10927) in cynomolgus monkey serum plotted versus electro-chemiluminescent signal in arbitrary units (MSD RFU) measured using the intact drug assay format depicted in FIG. 5B.

FIG. 7C is a line graph of various concentrations of GalNAc-conjugated RNAi constructs (construct nos. 1907, 7213, 8172, and 10927) in rat serum plotted versus electro-chemiluminescent signal in arbitrary units (MSD RFU) measured using the intact drug assay format depicted in FIG. 5B.

FIG. 7D is an overlay of the graphs depicted in FIGS. 7A-7C.

FIG. 8 is a line graph of various concentrations of the indicated GalNAc-conjugated RNAi constructs or their component sense and antisense strands in mouse serum plotted versus electro-chemiluminescent signal in arbitrary units (MSD RFU) measured using the intact drug assay format depicted in FIG. 5B.

FIG. 9 is a graph showing the relationship between concentration of monoclonal antibody-RNAi construct conjugate molecules in mouse serum and electro-chemiluminescent signal in arbitrary units (MSD RFU). The same RNAi construct was conjugated to a human monoclonal antibody recognizing a cell-surface receptor at different conjugation sites within the antibody at an RNA-to-antibody ratio of 1 or 2. The conjugate molecules were evaluated in two different immunoassays. In assay 1, the 14K10 pan-specific RNAi construct antibody was used as a capture reagent and a ruthenium-labeled anti-human Fc antibody was used as the detection reagent (see format depicted in FIG. 5C). Assay 2 was of a similar format except that the anti-GalNAc moiety antibody 14D4 was used as the capture reagent in place of the 14K10 antibody. A ruthenium-labeled anti-human Fc antibody was also used for detection in assay 2.

FIG. 10A is a line graph of the relationship between serum concentration of total drug (“total”) or intact mAb-RNAi conjugate molecule (“intact”) over time in mice receiving the mAb-RNAi conjugate molecules 15722 or 15723 intravenously. Mice received a single dose of 12 mg/kg of either the 15722 conjugate molecule or 15723 conjugate molecule at time 0 and serum samples were taken at various time points following administration of the conjugate molecules. RAR=RNA-to-antibody ratio.

FIG. 10B is a line graph of the relationship between concentration in the pancreas of total drug (“total”) or intact mAb-RNAi conjugate molecule (“intact”) over time in mice receiving the mAb-RNAi conjugate molecules 15722 or 15723 intravenously. Mice received a single dose of 12 mg/kg of either the 15722 conjugate molecule or 15723 conjugate molecule at time 0 and pancreas samples were taken at various time points following administration of the conjugate molecules. RAR=RNA-to-antibody ratio.

FIG. 10C is a line graph of the relationship between concentration in the liver of total drug (“total”) or intact mAb-RNAi conjugate molecule (“intact”) over time in mice receiving the mAb-RNAi conjugate molecules 15722 or 15723 intravenously. Mice received a single dose of 12 mg/kg of either the 15722 conjugate molecule or 15723 conjugate molecule at time 0 and liver samples were taken at various time points following administration of the conjugate molecules. RAR=RNA-to-antibody ratio.

FIG. 10D is a line graph of the relationship between concentration in the kidney of total drug (“total”) or intact mAb-RNAi conjugate molecule (“intact”) over time in mice receiving the mAb-RNAi conjugate molecules 15722 or 15723 intravenously. Mice received a single dose of 12 mg/kg of either the 15722 conjugate molecule or 15723 conjugate molecule at time 0 and kidney samples were taken at various time points following administration of the conjugate molecules. RAR=RNA-to-antibody ratio.

### DETAILED DESCRIPTION

The present invention is based, in part, on the generation of monoclonal antibodies that specifically bind to chemically-modified nucleic acids and novel immunoassays incorporating such monoclonal antibodies. The monoclonal antibodies of the invention are useful for a variety of applications, including detection of nucleic acid-based drugs in biological fluids and tissues, for example, to assess the pharmacokinetic properties, metabolism, and distribution of such nucleic acid-based drugs.

Nucleic acids are notoriously poor immunogens and it is difficult to generate antibodies to specific nucleic acids (see e.g., Hu et al., Expert Rev. Mol. Diagn., Vol. 14: 895-916, 2014 and Feederle and Schepers, RNA Biology, Vol. 14: 1089-1098, 2017). Most antibodies that can recognize modified nucleotides are directed to single modified nucleotides and do not always recognize the modified nucleotide when incorporated into polynucleotide chains (Feederle and Schepers, 2017). The present invention provides a novel method for generating monoclonal antibodies that specifically bind to chemically-modified nucleic acid molecules using a multivalent nucleic acid-displaying nanobead as an immunogen. This method produces significantly greater antigen-specific antibody titers as well as antibodies with varied binding specificity as compared to methods employing the conventional immunogen comprised of the nucleic acid linked to a carrier protein (see Example 1). Moreover, the method is able to generate rabbit monoclonal antibodies, which are more difficult to obtain and less readily available (see Feederle and Schepers, 2017). In some embodiments, the method comprises conjugating a plurality of nucleic acid molecules to a bead to form an immunogen, wherein the nucleic acid molecules each comprise one or more modified nucleotides; administering the immunogen to an animal; obtaining splenocytes from the immunized animal; selecting splenocytes that are IgG positive and bind to the chemically-modified nucleic acid molecules thereby isolating antigen-specific antibody producing cells; plating the antigen-specific antibody producing cells in single-cell culture; and isolating the monoclonal antibody from the single-cell culture.

The methods of the invention can be used to generate monoclonal antibodies against any type of nucleic acid molecule. The term “nucleic acid molecule” refers to a molecule comprising polymer(s) of nucleotides, such as polynucleotides and oligonucleotides. The nucleic acid molecule can comprise ribonucleotides, dexoyribonucleotides, modified nucleotides, or combinations thereof. The nucleic acid molecule can be single-stranded, double-stranded, or comprise both single-stranded and double-stranded regions. In some embodiments, the nucleic acid molecule is an antisense oligonucleotide having a sequence complementary to a region of a target gene or mRNA sequence. In other embodiments, the nucleic acid molecule is an anti-miRNA oligonucleotide (e.g. antagomir or antimiR) having a sequence complementary to a miRNA. In still other embodiments, the nucleic acid molecule is a messenger RNA (mRNA) or fragment thereof. In certain embodiments, the nucleic acid molecule is an RNAi construct. As used herein, the term “RNAi construct” refers to an agent comprising a nucleic acid molecule that is capable of downregulating expression of a target gene via an RNA interference mechanism when introduced into a cell. RNA interference is the process by which a nucleic acid molecule induces the cleavage and degradation of a target RNA molecule (e.g. mRNA molecule) in a sequence-specific manner, e.g. through an RNA-induced silencing complex (RISC) pathway. In some embodiments, the RNAi construct comprises a double-stranded nucleic acid molecule comprising two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g. Watson-Crick, Hoogsteen or reversed Hoogsteen hydrogen bonding) between complementary bases in the two polynucleotides. The strand comprising a region having a sequence that is substantially complementary to a target sequence (e.g. target mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

A first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, about 85%, about 90%, about 95%, about 96%, about 97%, about 98%, or about 99% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized.

In some embodiments, a region of the antisense strand of the RNAi constructs comprises a sequence that is fully complementary to a region of a target gene sequence (e.g. target mRNA). In such embodiments, the sense strand of the RNAi construct may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

In certain embodiments, the RNAi construct comprises a sense strand and an antisense strand, which are two separate molecules that hybridize to form a duplex region but are otherwise unconnected. Such double-stranded nucleic acid molecules formed from two separate strands are referred to as “small interfering RNAs” or “short interfering RNAs” (siRNAs). Thus, in some embodiments, the RNAi constructs comprise an siRNA. In related embodiments, the RNAi constructs comprise a microRNA (miRNA) or a miRNA mimetic. MiRNAs are endogenous double-stranded RNA molecules that regulate gene expression through the RNA interference pathway.

In other embodiments, the RNAi construct comprises a nucleic acid molecule having partially self-complementary regions that hybridize to form a duplex region, i.e. the sense and antisense strands are part of a self-complementary region of a single nucleic acid molecule. Such nucleic acid molecules with at least partially self-complementary regions are referred to as “short hairpin RNAs” (shRNAs). shRNA molecules typically comprise a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. Thus, in certain embodiments, the RNAi constructs comprise a shRNA. In related embodiments, the RNAi constructs comprise a precursor miRNA (pre-miRNA).

The length of the nucleic acid molecule will vary depending on the type of nucleic acid molecule. For purposes of preparing the immunogen as described in more detail below, the nucleic acid molecule will generally be from about 15 nucleotides in length to about 150 nucleotides in length. For instance, each strand of a double-stranded siRNA molecule, a miRNA molecule, or a miRNA mimetic molecule is typically about 15 nucleotides in length to about 30 nucleotides in length, whereas as a single-stranded shRNA molecule and a pre-miRNA molecule, which fold back on themselves to form stem-loop or hairpin structures, can be from about 35 nucleotides to about 120 nucleotides in length. Antisense oligonucleotides and anti-miRNA oligonucleotides are typically from about 15 nucleotides to about 25 nucleotides in length. In certain embodiments, the nucleic acid molecule is an RNAi construct comprising a sense strand and an antisense strand, wherein each strand is independently about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length.

In embodiments in which the RNAi construct comprises an siRNA, the sense strand and antisense strand need not be the same length as the length of the duplex region. A “duplex region” refers to the region in two complementary or substantially complementary polynucleotides that form base pairs with one another, either by Watson-Crick base pairing or other hydrogen bonding interaction, to create a duplex between the two polynucleotides. For instance, one or both strands may be longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. In certain other embodiments, the nucleotide overhang comprises a single nucleotide.

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e. the molecule is double-stranded over its entire length).

The nucleic acid molecules are preferably chemically-modified nucleic acid molecules, which refers to nucleic acid molecules comprising one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate, or deoxyribonucleotides containing deoxyadenosine monophosphate, deoxyguanosine monophosphate, deoxythymidine monophosphate, and deoxycytidine monophosphate. However, the nucleic acid molecules used in the methods of the invention may comprise combinations of modified nucleotides, deoxyribonucleotides, and ribonucleotides. In certain embodiments, all the nucleotides in the nucleic acid molecules are modified nucleotides. In other embodiments, all the nucleotides in the nucleic acid molecules are a combination of modified nucleotides and deoxyribonucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than H or OH. Such 2′-modifications include, but are not limited to, 2′-O-alkyl (e.g. O—C₁-C₁₀ or O—C₁-C₁₀ substituted alkyl), 2′-O-allyl (O—CH₂CH═CH₂), 2′-C-allyl, 2′-deoxy-2′-fluoro (also referred to as 2′-F or 2′-fluoro), 2′-O-methyl (OCH₃), 2′-O-methoxyethyl (O—(CH₂)₂OCH₃), 2′-OCF₃, 2′-O(CH₂)₂SCH₃, 2′-O-aminoalkyl, 2′-amino (e.g. NH₂), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA; Aminooxy (4′-CH₂—O—N(R)-2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)-2′) BNA; propylene carbocyclic (4′-(CH₂)₃-2′) BNA; and Methoxy(ethyleneoxy) (4′-CH(CH₂OMe)—O-2′) BNA (also referred to as constrained MOE or cMOE). These and other sugar-modified nucleotides that can be incorporated into nucleic acid molecules for use in the methods of the invention are described in U.S. Pat. No. 9,181,551, U.S. Patent Publication No. 2016/0122761, and Deleavey and Damha, Chemistry and Biology, Vol. 19: 937-954, 2012, all of which are hereby incorporated by reference in their entireties.

In some embodiments, the nucleic acid molecules comprise one or more 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNAs), or combinations thereof. In certain embodiments, the nucleic acid molecules comprise one or more 2′-fluoro modified nucleotides, 2′-O-methyl modified nucleotides, 2′-O-methoxyethyl modified nucleotides, or combinations thereof. In certain embodiments, the nucleic acid molecules comprise one or more 2′-fluoro modified nucleotides, 2′-O-methyl modified nucleotides or combinations thereof.

In certain embodiments, the modified nucleotides incorporated into the nucleic acid molecules for use in the methods of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the nucleic acid molecules for use in the methods of the invention include those described in Herdewijn, Antisense Nucleic Acid Drug Dev., Vol. 10: 297-310,2000 and Peacock et al., J. Org. Chem., Vol. 76: 7295-7300, 2011, both of which are hereby incorporated by reference in their entireties. The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the nucleic acid molecules may comprise one or more abasic nucleotides. An “abasic nucleotide” or “abasic nucleoside” is a nucleotide or nucleoside that lacks a nucleobase at the 1′ position of the ribose sugar. In certain embodiments, the abasic nucleotides are incorporated into one or both the terminal ends of the nucleic acid molecule, for example, at the terminal ends of the sense and/or antisense strands of RNAi constructs. In one embodiment, the sense strand comprises an abasic nucleotide as the terminal nucleotide at its 3′ end, its 5′ end, or both its 3′ and 5′ ends. In another embodiment, the antisense strand comprises an abasic nucleotide as the terminal nucleotide at its 3′ end, its 5′ end, or both its 3′ and 5′ ends. In such embodiments in which the abasic nucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage. Abasic nucleotides may also comprise a sugar modification, such as any of the sugar modifications described above. In certain embodiments, abasic nucleotides comprise a 2′-modification, such as a 2′-fluoro modification, 2′-O-methyl modification, or a 2′-H (deoxy) modification. In one embodiment, the abasic nucleotide comprises a 2′-O-methyl modification. In another embodiment, the abasic nucleotide comprises a 2′-H modification (i.e. a deoxy abasic nucleotide).

The chemically-modified nucleic acid molecules for use in or detected according to the methods of the invention may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the chemically-modified nucleic acid molecules for use in the methods of the invention are described in U.S. Pat. Nos. 6,693,187, 9,181,551, U.S. Patent Publication No. 2016/0122761, and Deleavey and Damha, Chemistry and Biology, Vol. 19: 937-954, 2012, all of which are hereby incorporated by reference in their entireties.

In certain embodiments, the chemically-modified nucleic acid molecules comprise one or more phosphorothioate internucleotide linkages. In embodiments in which the nucleic acid molecule is double-stranded (e.g. RNAi constructs comprising a sense strand and antisense strand), the phosphorothioate internucleotide linkages may be present in the sense strand, antisense strand, or both strands. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. Double-stranded nucleic acid molecules can comprise one or more phosphorothioate internucleotide linkages at the 3′-end, the 5′-end, or both the 3′- and 5′-ends of the sense strand, the antisense strand, or both strands. For instance, in certain embodiments in which the nucleic acid molecule is an RNAi construct comprising a sense strand and an antisense strand, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

Chemically-modified nucleic acid molecules, including RNAi constructs, can readily be made using techniques known in the art, for example, using conventional nucleic acid solid phase synthesis. Polynucleotides can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA).

A 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion sources are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Preferred catalysts include, e.g., tetrazole, S-ethyl-tetrazole, benzylthiotetrazole, p-nitrophenyltetrazole.

As can be appreciated by the skilled artisan, further methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the chemically-modified nucleic acid molecules described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of chemically-modified nucleic acid molecules is also available from several commercial vendors, including Dharmacon, Inc. (Lafayette, CO), AxoLabs GmbH (Kulmbach, Germany), and Ambion, Inc. (Foster City, CA).

The nucleic acid molecules may be covalently linked to a ligand. As used herein, a “ligand” refers to any compound or molecule that is capable of interacting with another compound or molecule, directly or indirectly. The interaction of a ligand with another compound or molecule may elicit a biological response (e.g. initiate a signal transduction cascade, induce receptor-mediated endocytosis) or may just be a physical association. The ligand can modify one or more properties of the nucleic acid molecule to which is attached, such as the pharmacodynamic, pharmacokinetic, binding, absorption, cellular distribution, cellular uptake, endosomal release, charge and/or clearance properties of the nucleic acid molecule.

The ligand may comprise a serum protein (e.g., human serum albumin, low-density lipoprotein, globulin), a cholesterol moiety, a vitamin (biotin, vitamin E, vitamin B₁₂), a folate moiety, a steroid, a bile acid (e.g. cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or antibody or binding fragment thereof (e.g. antibody or binding fragment that targets the nucleic acid molecule to a specific cell type, such as liver). Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, O3-(oleoyl)lithocholic acid, O3-(oleoyl)cholenic acid, dimethoxytrityl, or phenoxazine), peptides (e.g., antennapedia peptide, Tat peptide, RGD peptides), alkylating agents, polymers, such as polyethylene glycol (PEG)(e.g., PEG-40K), polyamino acids, and polyamines (e.g. spermine, spermidine).

In some embodiments, the ligand comprises a lipid or other hydrophobic molecule. In one embodiment, the ligand comprises a cholesterol moiety or other steroid. Cholesterol-conjugated oligonucleotides have been reported to be more active than their unconjugated counterparts (Manoharan, Antisense Nucleic Acid Drug Development, Vol. 12: 103-228, 2002). Ligands comprising cholesterol moieties and other lipids for conjugation to nucleic acid molecules have also been described in U.S. Pat. Nos. 7,851,615; 7,745,608; and 7,833,992, all of which are hereby incorporated by reference in their entireties. In another embodiment, the ligand comprises a folate moiety. Polynucleotides conjugated to folate moieties can be taken up by cells via a receptor-mediated endocytosis pathway. Such folate-polynucleotide conjugates are described in U.S. Pat. No. 8,188,247, which is hereby incorporated by reference in its entirety.

In certain embodiments, the ligand may target the nucleic acid molecule to a specific tissue or cell type, for example, where it is desirable to restrict the activity of the nucleic acid molecule to that specific tissue or cell type. In one embodiment, the ligand targets delivery of the nucleic acid molecule specifically to liver cells (e.g. hepatocytes) using various approaches as described in more detail below. In certain embodiments, the nucleic acid molecules (e.g. RNAi constructs or antisense oligonucleotides) are targeted to liver cells with a ligand that binds to the surface-expressed asialoglycoprotein receptor (ASGR) or component thereof (e.g. ASGR1, ASGR2).

In some embodiments, the ligand covalently linked to the nucleic acid molecule comprises a carbohydrate. A “carbohydrate” refers to a compound made up of one or more monosaccharide units having at least 6 carbon atoms (which can be linear, branched or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In certain embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In some embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells because such ligands bind to the ASGR expressed on the surface of hepatocytes. See, e.g., D'Souza and Devarajan, J. Control Release, Vol. 203: 126-139, 2015. Examples of GalNAc- or galactose-containing ligands that can be covalently linked to the nucleic acid molecules used in the methods of the invention are described in U.S. Pat. Nos. 7,491,805; 8,106,022; and 8,877,917; U.S. Patent Publication No. 20030130186 and 20170253875; and WIPO Publication Nos. WO 2013166155, WO 2014179620, and WO 2018039647, all of which are hereby incorporated by reference in their entireties.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety can be bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent GalNAc-containing ligands that can be linked to the nucleic acid molecules used in the methods of the invention are described in Example 1 (TL01, TL02, and TL03 GalNAc moieties). Other examples of trivalent and tetravalent galactose and GalNAc-containing ligands that can be linked to the nucleic acid molecules used in the methods of the invention have been described previously. See, e.g., U.S. Pat. Nos. 7,491,805 and 8,106,022; U.S. Patent Publication No. 20170253875; and WIPO Publication Nos. WO 2013166155, WO 2014179620, and WO 2018039647.

The ligand can be attached or linked to the nucleic acid molecule directly or indirectly via a linker moiety. The ligand can be attached to nucleobases, pentose sugars, or internucleotide linkages of the nucleic acid molecule. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2-, 5-, and 6-positions of a pyrimidine nucleobase can be attached to a ligand. Conjugation or attachment to the pentose sugars of nucleotides can occur at any carbon atom. Exemplary carbon atoms of a pentose sugar that can be attached to a ligand include the 2′, 3′, and 5′ carbon atoms. The 1′ position can also be attached to a ligand where the nucleobase is omitted, such as in an abasic nucleotide. Internucleotide linkages can also support ligand attachments. For phosphorus-containing linkages (e.g., phosphodiester, phosphorothioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In certain embodiments, the ligand may be attached to the 3′ or 5′ end of the nucleic acid molecule. For instance, in embodiments in which the nucleic acid molecule is double stranded, the ligand can be attached to the 3′ or 5′ end of either strand, e.g. the sense strand or antisense strand of an RNAi construct. In some such embodiments, the ligand is covalently attached to the 5′ end of the sense strand. In such embodiments, the ligand is attached to the 5′-terminal nucleotide of the sense strand. In these and other embodiments, the ligand is attached at the 5′-position of the 5′-terminal nucleotide of the sense strand. In embodiments in which an inverted abasic nucleotide or inverted deoxyribonucleotide is the 5′-terminal nucleotide of the sense strand and linked to the adjacent nucleotide via a 5′-5′ internucleotide linkage, the ligand can be attached at the 3′-position of the inverted abasic nucleotide or inverted deoxyribonucleotide. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In embodiments in which an inverted abasic nucleotide or inverted deoxyribonucleotide is the 3′-terminal nucleotide of the sense strand and linked to the adjacent nucleotide via a 3′-3′ internucleotide linkage, the ligand can be attached at the 5′-position of the inverted abasic nucleotide or inverted deoxyribonucleotide. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand. In other embodiments, the ligand is attached at the 2′-position of the sugar of the 5′-terminal nucleotide of the sense strand.

In certain embodiments, the ligand is attached to the nucleic acid molecule via a linker moiety. A “linker moiety” is an atom or group of atoms that covalently joins a ligand to the nucleic acid molecule. The linker moiety may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker moiety may comprise a bifunctional linking moiety, which generally comprises an alkyl moiety with two functional groups. One of the functional groups is selected to bind to the nucleic acid molecule and the other is selected to bind essentially any selected group, such as a ligand as described herein. In certain embodiments, the linker moiety comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like. Linker moieties that may be used to attach a ligand to the nucleic acid molecule include, but are not limited to, pyrrolidine, 8-amino-3,6-dioxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl.

Other types of linker moieties suitable for attaching ligands to nucleic acid molecules are known in the art and can include the linker moieties described in U.S. Pat. Nos. 7,723,509; 8,017,762; 8,828,956; 8,877,917; and 9,181,551, all of which are hereby incorporated by reference in their entireties.

In certain embodiments, the ligand covalently attached to the nucleic acid molecule comprises a GalNAc moiety, e.g, a multivalent GalNAc moiety. In some embodiments, the multivalent GalNAc moiety is a trivalent GalNAc moiety and is attached to the 3′ end of the nucleic acid molecule (e.g. at the 3′ end of the sense strand of an RNAi construct). In other embodiments, the multivalent GalNAc moiety is a trivalent GalNAc moiety and is attached to the 5′ end of the nucleic acid molecule (e.g. at the 5′ end of the sense strand of an RNAi construct). In yet other embodiments, the multivalent GalNAc moiety is a tetravalent GalNAc moiety and is attached to the 3′ end of the nucleic acid molecule (e.g. at the 3′ end of the sense strand of an RNAi construct). In still other embodiments, the multivalent GalNAc moiety is a tetravalent GalNAc moiety and is attached to the 5′ end of the nucleic acid molecule (e.g. at the 5′ end of the sense strand of an RNAi construct).

In certain embodiments, the nucleic acid molecules used in or detected according to the methods of the invention are covalently linked to a ligand comprising the following structure, where “Ac” represents an acetyl group:

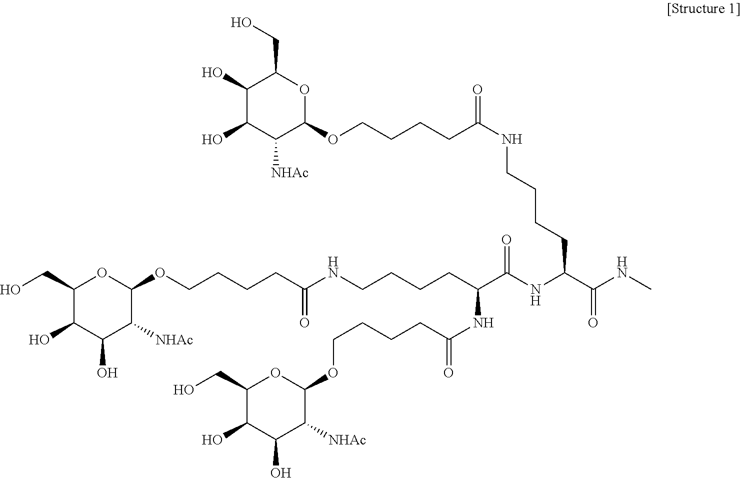 [SMILES: CNC(=O)[C@H](CCCCNC(=O)CCCCO[C@@H]1O[C@H](CO)[C@H](O)[C@H](O)[C@H]1NC(C)=O)NC(=O)[C@H](CCCCNC(=O)CCCCO[C@@H]1O[C@H](CO)[C@H](O)[C@H](O)[C@H]1NC(C)=O)NC(=O)CCCCO[C@@H]1O[C@H](CO)[C@H](O)[C@H](O)[C@H]1NC(C)=O]

In preferred embodiments, the ligand comprising this structure is covalently attached to the 5′ end of the nucleic acid molecule (e.g. at the 5′ end of the sense strand of an RNAi construct) via a linker moiety, such as the linker moieties described herein.

In one embodiment, the nucleic acid molecules used in or detected according to the methods of the invention are covalently attached to the TL01 GalNAc moiety. In another embodiment, the nucleic acid molecules used in or detected according to the methods of the invention are covalently attached to the TL02 GalNAc moiety. In yet another embodiment, the nucleic acid molecules used in or detected according to the methods of the invention are covalently attached to the TL03 GalNAc moiety. The structures of the TL01, TL02, and TL03 GalNAc moieties are provided in Example 1.

In certain embodiments, the methods of the invention for generating monoclonal antibodies to chemically-modified nucleic acid molecules employ a multivalent nucleic acid-displaying nanobead as an immunogen. In some such embodiments, the methods comprise conjugating a plurality of chemically-modified nucleic acid molecules to a bead to form the immunogen. A plurality of chemically-modified nucleic acid molecules refers to more than two molecules, typically 10 or more, 50 or more, 100 or more, 500 or more, or 1000 or more molecules. The bead can be made from any number of materials including, but not limited to, latex, polystyrene, polypropylene, polycarbonate, polyvinylidene fluoride, silica, or other polymer having properties similar to any of the foregoing polymers. In certain embodiments, the bead is a polystyrene bead. In other embodiments, the bead is a silica bead. The average diameter of the bead can be from about 20 nm to about 5 μm or from about 50 nm to 2 μm. In preferred embodiments, the bead has an average diameter of at least 70 nm. Without being bound by theory, it is believed that beads of at least this size or greater are likely to have a decreased in vivo clearance rate thereby increasing the exposure of the immunogen to the immune system. In some embodiments, the bead has an average diameter of about 0.1 μm to about 5 μm. In other embodiments, the bead has an average diameter of about 0.1 μm to about 1 μm. In one embodiment, the bead has an average diameter of about 0.1 μm.

The chemically-modified nucleic acid molecules can be conjugated to the beads through various methods known to those of skill in the art, such as covalent coupling, adsorption, and affinity binding. The chemically-modified nucleic acid molecules can be coupled directly to the beads via functional groups present on the surface the bead. Suitable functional groups can include carboxyl, amino, hydroxyl, hydrazide, and chloromethyl groups for polymeric beads and silanol and carboxyl groups for silica beads. 1-Ethyl-3-(3-dimethylaminopropyl) carbodiimide (EDAC)-mediated coupling is often used for the covalent immobilization of nucleic acid molecules on carboxyl-functionalized beads. Linkers can be employed to act as spacers between the nucleic acid molecules and the surface of the bead. Suitable linkers are described in the art (see, e.g., Hermanson, Bioconjugate techniques, San Diego: Academic Press (1996); Jones, IVD Technology, Vol. Nov./Dec. 39, 2001; and Carmon et al., BioTechniques, Vol. 32: 410-420, 2002). Nucleic acid molecules can also be passively adsorbed to the surface of silica beads having hydroxyl or silanol groups. Methods of covalently coupling nucleic acid molecules to solid supports are known to those of skill in the art and include methods described in Andreadis and Chrisey, Nucleic Acids Res., Vol. 28: e5, 2000; Armstrong et al., Cytometry, Vol 20: 102-108, 2000; Spiro et al., Appl. Environ. Microbiol., Vol. 66: 4258-4265, 2000; Beaucage, Curr. Med. Chem., Vol. 8: 1213-1244, 2001; Taylor et al., BioTechniques, Vol. 30: 661-666, 668-669, 2001; and Walsh et al., J. Biochem. Biophys. Methods, Vol. 47: 221-231, 2001.

Alternatively, the chemically-modified nucleic acid molecules can be coupled to beads via an affinity binding interaction. For example, the bead can be coated with an affinity binding protein (e.g. streptavidin), that interacts with a binding partner (e.g. biotin) coupled to the nucleic acid molecule. In certain embodiments, the bead is coated with streptavidin and the chemically-modified nucleic acid molecule is biotinylated (e.g. at the 5′ end and/or 3′ end of one or both strands). Streptavidin-coated beads and beads containing various functional groups as described herein suitable for use in the methods of the invention are commercially available from several vendors, including the polystyrene and silica microspheres from Bangs Laboratories, Inc.

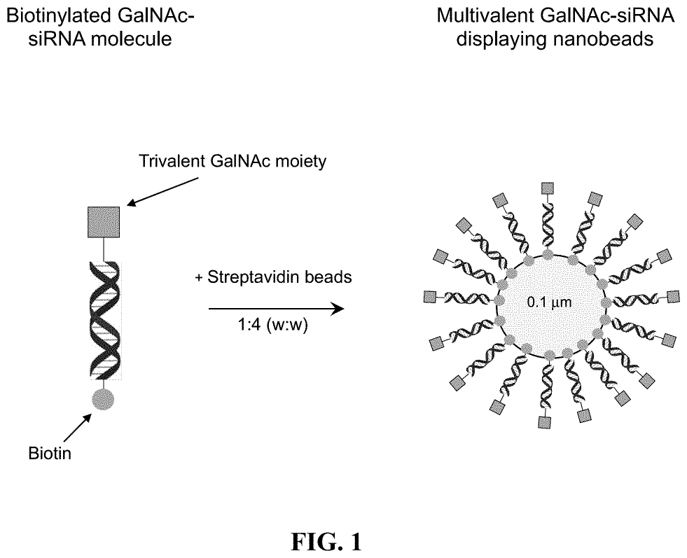

The chemically-modified nucleic acid molecules can be conjugated to the beads in different orientations. In embodiments in which the chemically-modified nucleic acid molecules are covalently linked to a ligand, the nucleic acid molecules are preferably conjugated to the bead at a position within the molecule distant from the ligand such that the ligand is positioned away from the surface of the bead. By way of example, a nucleic acid molecule that is covalently linked to a ligand at its 5′ end would preferably be conjugated to the bead at its 3′ end. In embodiments in which the nucleic acid molecule is double-stranded (e.g. an RNAi construct comprising a sense strand and an antisense strand), either strand can be conjugated to the bead at either its 5′ end or 3′ end. In one embodiment, the sense strand is conjugated to the bead at its 5′ end. In another embodiment, the antisense strand is conjugated to the bead at its 5′ end. In yet another embodiment, the sense strand is conjugated to the bead at its 3′ end. In still another embodiment, the antisense strand is conjugated to the bead at its 3′ end. In certain embodiments, the nucleic acid molecule is an RNAi construct comprising a sense strand and an antisense strand, wherein the sense strand is covalently linked to a ligand (e.g. a GalNAc-containing ligand) at its 5′ end and the antisense strand is conjugated to the bead (e.g. via biotin) at its 5′ end. In certain other embodiments, the nucleic acid molecule is an RNAi construct comprising a sense strand and an antisense strand, wherein the sense strand is covalently linked to a ligand (e.g. a GalNAc-containing ligand) at its 3′ end and the antisense strand is conjugated to the bead (e.g. via biotin) at its 3′ end. An exemplary orientation of GalNAc-conjugated RNAi constructs on a bead is shown in FIG. 1.

The density of the chemically-modified nucleic acid molecules on the beads may vary by the type of nucleic acid molecule used, for example, with a higher density for nucleic acid molecules having a lower molecular weight and a lower density for nucleic acid molecules having a higher molecular weight. Suitable densities of the chemically-modified nucleic acid molecules on the beads can be from about 10² molecules/μm² to about 10⁶ molecules/μm², from about 10⁴ molecules/μm² to about 10⁶ molecules/μm², from about 10³ molecules/μm² to about 10⁵ molecules/μm², or from about 5×10⁴ molecules/μm² to about 8×10⁵ molecules/μm². For embodiments in which the chemically-modified nucleic acid molecules are double-stranded RNAi constructs (e.g. siRNA molecules), a bead having a diameter of about 0.1 μm may have about 2,000 to 6,400 RNAi constructs conjugated thereto.

Following preparation of the immunogen, according to some embodiments of the methods of the invention, the immunogen is administered to an animal to generate an immune response to the immunogen. Any immunocompetent animal can be used, such as a mouse, rabbit, rat, goat, non-human primate (e.g. cynomolgus or rhesus monkey), or other mammal. In certain embodiments of the methods of the invention, the animal administered the immunogen is a rabbit. In certain other embodiments of the methods of the invention, the animal administered the immunogen is a mouse. Next, splenocytes are obtained from the immunized animal and selected for IgG surface expression (i.e. IgG positive) and ability to bind to the chemically-modified nucleic acid molecule used in the immunogen to identify antigen-specific antibody producing cells. Splenocytes that are IgG positive and antigen-specific can be identified using a labeled version of the antigen (e.g. fluorescently-labeled chemically-modified nucleic acid molecule) and a labeled (e.g. fluorescently-labeled) anti-IgG antibody specific for IgG molecules from the immunized animal (e.g. anti-rabbit IgG antibody). The cells that are positive for both labels (i.e. antigen-specific antibody producing cells) can then be plated in single-cell culture using methods known in the art, such as limiting dilution, fluorescence activated cell sorting, and microfluidic techniques. In certain embodiments of the methods of the invention, the antigen-specific antibody producing cells are selected and plated in single-cell culture using a fluorescence activated cell sorting technique, such as that described in Example 1 herein.

The monoclonal antibodies can be isolated from the single-cell culture and optionally purified using any known technique in the art, such as protein A chromatography. The monoclonal antibodies can also be optionally screened to confirm or further characterize the binding properties of the antibodies, for example, using ELISA or competition-binding assays as described in Example 1 herein. In some embodiments, the methods further comprise lysing the B-cells derived from the single-cell culture and sequencing the antibody genes from the clonal B cells. The sequences can then be used to recombinantly produce the monoclonal antibody in cell culture as further described herein.

In alternative embodiments of the methods of the invention, hybridomas can be generated from splenocytes obtained from the animals immunized with the bead-based immunogen described herein. For instance, a hybridoma cell line is produced by immunizing an animal (e.g., a rabbit, rat, mouse, or other mammal) with a bead-based immunogen comprising a plurality of chemically-modified nucleic acid molecules of interest as described herein; harvesting splenocytes from the immunized animal; fusing the harvested splenocytes to a myeloma cell line, thereby generating hybridoma cells; establishing hybridoma cell lines from the hybridoma cells, and identifying a hybridoma cell line that produces an antibody that binds to the chemically-modified nucleic acid molecules of interest. Methods of generating hybridoma cell lines by fusing splenocytes obtained from the immunized animals with myeloma cells are known in the art. See, e.g., Antibodies; Harlow and Lane, Cold Spring Harbor Laboratory Press, 1st Edition, e.g. from 1988, or 2nd Edition, e.g. from 2014. Myeloma cells for use in hybridoma-producing fusion procedures preferably are non-antibody-producing, have high fusion efficiency, and enzyme deficiencies that render them incapable of growing in certain selective media, which support the growth of only the desired fused cells (hybridomas). Examples of suitable cell lines for use in fusions with mouse cells include, but are not limited to, Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul. Examples of suitable cell lines used for fusions with rat cells include, but are not limited to, R210.RCY3, Y3-Ag 1.2.3, IR983F and 4B210. Other cell lines useful for cell fusions are U-266, GM1500-GRG2, LICR-LON-HMy2 and UC729-6.

The present invention includes monoclonal antibodies produced by any of the methods described herein. The monoclonal antibodies find use in a variety of applications, including detection and isolation of chemically-modified nucleic acid molecules in biological fluids and tissues, for example, using immunoassay, immunoprecipitation, and immunohistochemistry techniques. The detection methods employing the monoclonal antibodies of the invention can be used, for example, to assess the pharmacokinetic properties (e.g. bioavailability), metabolism, and distribution of therapeutic molecules comprising chemically-modified nucleic acids. Labeled forms of the monoclonal antibodies of the invention can be used in competitive assay formats to detect antibodies against nucleic acid-based drugs in samples from subjects administered nucleic acid-based drugs. The monoclonal antibodies of the invention can also be used as positive controls in such anti-drug antibody assays.

In some embodiments, the present invention provides antibodies that specifically bind to chemically-modified nucleic acid molecules independent of nucleotide sequence (e.g. pan-specific antibodies). Such antibodies have binding specificity for chemically-modified double-stranded nucleic acid molecules and do not significantly bind or cross-react with endogenous nucleic acid molecules. In some embodiments, the antibodies have a greater binding affinity for double-stranded nucleic acid molecules as compared to single-stranded nucleic acid molecules.

In certain embodiments, the present invention provides antibodies that bind in a sequence specific manner to an RNAi construct comprising the nucleotide sequence of SEQ ID NO: 192. Such antibodies specifically bind to the 1851 RNAi construct molecule described herein and do not significantly bind or cross-react with other chemically-modified nucleic acid molecules having different nucleotide sequences. In other embodiments, the present invention provides antibodies that specifically bind to a N-acetyl-galactosamine (GalNAc) moiety, such as those described herein. In some such embodiments, the GalNAc moiety is a multivalent GalNAc moiety. In one embodiment, the GalNAc moiety is a trivalent GalNAc moiety. In another embodiment, the GalNAc moiety has the structure of Structure 1.

An antibody is a protein that comprises an antigen-binding fragment that specifically binds to an antigen, and a scaffold or framework portion that allows the antigen-binding fragment to adopt a conformation that promotes binding of the antibody to the antigen. As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (p), delta (A), gamma (γ), alpha (a), and epsilon (c), and define the antibody's isotype as IgM, IgD, IgG, IgA, and IgE, respectively. In some species (e.g. humans), the IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes.

The present invention also includes antigen-binding fragments of the monoclonal antibodies described herein. An “antigen-binding fragment,” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab)₂ fragment, a Fv fragment, a Fd fragment, and a complementarity determining region (CDR) fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

The term “isolated molecule” (where the molecule is, for example, a polypeptide, a polynucleotide, a nucleic acid molecule, an antibody, or antigen-binding fragment) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

An antibody or antigen-binding fragment “specifically binds” to a target antigen when it has a significantly higher binding affinity for, and consequently is capable of distinguishing, that antigen compared to its affinity for other unrelated molecules, under similar binding assay conditions. Antibodies or antigen-binding fragments that specifically bind an antigen may have an equilibrium dissociation constant (K_{D})≤1×10⁻⁶ M. The antibody or antigen-binding fragment specifically binds antigen with “high affinity” when the K_{D} is ≤1×10⁻⁸ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and kₐ) and affinity (K_{D}) constants can be calculated in real-time using the bio-layer interferometry method. In some embodiments, the antibodies or antigen-binding fragments described herein exhibit desirable characteristics such as binding avidity as measured by kₐ (dissociation rate constant) for their respective target antigens of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for their respective target antigens of about 10⁻⁷, 10⁻⁸, 10⁻⁹, 10⁻¹⁰, 10⁻¹¹, 10⁻¹² M or lower (lower values indicating higher binding affinity).

The antibodies or antigen-binding fragments of the invention may comprise one or more complementarity determining regions (CDR) from the light and heavy chain variable regions of the monoclonal antibodies described herein. The term “CDR” refers to the complementarity determining region (also termed “minimal recognition units” or “hypervariable region”) within antibody variable sequences. There are three heavy chain variable region CDRs (CDRH1, CDRH2 and CDRH3) and three light chain variable region CDRs (CDRL1, CDRL2 and CDRL3). The term “CDR region” as used herein refers to a group of three CDRs that occur in a single variable region (i.e. the three light chain CDRs or the three heavy chain CDRs). The CDRs in each of the two chains typically are aligned by the framework regions (FRs) to form a structure that binds specifically with a specific epitope or domain on the target antigen. From N-terminus to C-terminus, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

In certain embodiments, the antibodies or antigen-binding fragments of the invention comprise at least one light chain variable region comprising a CDRL1, CDRL2, and CDRL3, and at least one heavy chain variable region comprising a CDRH1, CDRH2, and CDRH3 from any of the monoclonal antibodies described herein. The monoclonal antibodies of the invention can be separated into the following three categories based on binding specificity: (i) monoclonal antibodies that specifically bind to chemically-modified nucleic acid molecules independent of nucleotide sequence (“pan-specific mAbs”), (ii) monoclonal antibodies that bind in a sequence specific manner to an RNAi construct comprising the nucleotide sequence of SEQ ID NO: 192 (“1851 RNAi construct-specific mAbs”), and (iii) monoclonal antibodies that specifically bind to a GalNAc moiety (“GalNAc moiety-specific mAbs”). Light chain and heavy chain variable regions and associated CDRs of exemplary monoclonal antibodies in each of these three categories are set forth below in Tables 1A and 1B, respectively.

TABLE 1A
Exemplary Light Chain Variable Region Amino Acid Sequences for Anti-Nucleic
Acid and Anti-GalNAc Moiety Antibodies

| | | | | |
|---|---|---|---|---|
| Antibody | | | | |
| ID. | VL Amino Acid Sequence | CDRL1 | CDRL2 | CDRL3 |

Pan-specific mAbs

| | | | | |
|---|---|---|---|---|
| 14K10 | ALVMTQTPSPVSAAVGGTVTI | QASQSVLNNNYLS | SASKLAT | AGYKNFGNDDNA |
| | NCQASQSVLNNNYLSWYQQK | (SEQ ID NO: 1) | (SEQ ID NO: | (SEQ ID NO: 25) |
| | PGQPPKLLIYSASKLATGVPSR | | 14) | |
| | FSGSGSGTQFTLTISGVQCDDA | | | |
| | ATYYCAGYKNFGNDDNAFGG | | | |
| | GTEVVVK (SEQ ID NO: 38) | | | |
| 14F4 | AQVLTQTSSPVSVNMGGTVTI | QSSLSVNRSDLS | LASNLES | AGGYSSGNDRNA |
| | NCQSSLSVNRSDLSWYQQKPG | (SEQ ID NO: 2) | (SEQ ID NO: | (SEQ ID NO: 26) |
| | QPPKLLIYLASNLESGVPSRFK | | 15) | |
| | GSGSGTHFTLTINGVECDDAA | | | |
| | TYYCAGGYSSGNDRNAFGGG | | | |
| | TEVVVK (SEQ ID NO: 39) | | | |
| 5I17 | ALVMTQTPSPVSAAVGGTVTI | QASQSVANNNYLA | QASTLAS | AGYRSYINADNA |
| | SCQASQSVANNNYLAWYQQK | (SEQ ID NO: 3) | (SEQ ID NO: | (SEQ ID NO: 27) |
| | PGQPPKLLIYQASTLASGVPSR | | 16) | |
| | FKGSGSGTQFTLTISGVECDDA | | | |
| | ATYYCAGYRSYINADNAFGG | | | |
| | GTEVVVE (SEQ ID NO: 40) | | | |

1851 RNAi construct-specific mAbs

| | | | | |
|---|---|---|---|---|
| 17K13 | AQVLTQTPSSVSAAVGGTVTI | QSSQSVWQKNWLS | RASTLAS | AGAYSSNSDVRA |
| | NCQSSQSVWQKNWLSWFQQK | (SEQ ID NO: 4) | (SEQ ID NO: | (SEQ ID NO: 28) |
| | PGQPPKLLIYRASTLASGVPSR | | 17) | |
| | FKSSGSGTQFTLTISGVQCDDA | | | |
| | ATYYCAGAYSSNSDVRAFGG | | | |
| | GTELVVK (SEQ ID NO: 41) | | | |
| 17F22 | AQVLTQTASPVSAAVGSTVTI | QASQSVYNNNNLA | YTSTLAS | QGEFACSTADCLT |
| | NCQASQSVYNNNNLAWYQQK | (SEQ ID NO: 5) | (SEQ ID NO: | (SEQ ID NO: 29) |
| | PGQPPKLLIYYTSTLASGVSSR | | 18) | |
| | FKGSGSGTQFTLTISGMQCDD | | | |
| | AATYYCQGEFACSTADCLTFG | | | |
| | GGTEVVVK (SEQ ID NO: 42) | | | |
| 20K24 | ALVMTQTPASVSAAVGGTVTI | QASESISSWLA | YASTLAS | AGYGSANDDKNG |
| | NCQASESISSWLAWYQQKPGQ | (SEQ ID NO: 6) | (SEQ ID NO: | (SEQ ID NO: 30) |
| | PPKLLIYYASTLASGVSSRFKG | | 19) | |
| | SGSRTEYTLTISDLECADAATY | | | |
| | YCAGYGSANDDKNGFGGGTE | | | |
| | VVVK (SEQ ID NO: 43) | | | |
| 20P19 | AYDMTQTPASVEVAVGGTVTI | QASQSINNELA | LAFTLAS | QQGYIISGVDNV |
| | KCQASQSINNELAWYQQKPG | (SEQ ID NO: 7) | (SEQ ID NO: | (SEQ ID NO: 31) |
| | QPPKLLIYLAFTLASGVPSRFK | | 20) | |
| | GSRSGTEFTLTISDLECADAAT | | | |
| | YYCQQGYIISGVDNVFGGGTE | | | |
| | VVVK (SEQ ID NO: 44) | | | |
| 19F24 | AQVLTQTPSSVSAAVGGTVTI | QSSQSVWEKNWLS | RASTLAS | AGAYSVNSDVRA |
| | NCQSSQSVWEKNWLSWFQQK | (SEQ ID NO: 8) | (SEQ ID NO: | (SEQ ID NO: 32) |
| | PGQPPKLLIYRASTLASGVPSR | | 17) | |
| | FKSSGSGTQFTLTISGVQCDDA | | | |
| | AVYYCAGAYSVNSDVRAFGG | | | |
| | GTELVVK (SEQ ID NO: 45) | | | |

GalNAc moiety-specific mAbs

| | | | | |
|---|---|---|---|---|
| 14D4 | AIVMTQTPSSKSVPVGGTVTIN | QSSESVYENNDLS | WASSLAS | AGYKSMSTDGFA |
| | CQSSESVYENNDLSWYQQKPG | (SEQ ID NO: 9) | (SEQ ID NO: | (SEQ ID NO: 33) |
| | QPPKLLIYWASSLASGVPSRFE | | 21) | |
| | GSGSGTQFTLTISNVVCDDAA | | | |
| | TYYCAGYKSMSTDGFAFGGG | | | |
| | TEVVVK (SEQ ID NO: 46) | | | |
| 16I3 | AIVMTQTPSSKSVPVGGTVTIS | QASQSLYKNTDLA | FASNLAS | AGYKSSTTDGFG |
| | CQASQSLYKNTDLAWFQQKP | (SEQ ID NO: 10) | (SEQ ID NO: | (SEQ ID NO: 34) |
| | GQPPKLLIYFASNLASGVTSRF | | 22) | |
| | KGSGSGTQFTLTISDVVCDDA | | | |
| | ATYYCAGYKSSTTDGFGFGGG | | | |
| | TEVVVK (SEQ ID NO: 47) | | | |
| 16A22 | AQVLAQTPSSVSAAVGGTVTI | QSSQSVGNNAYLS | YASTLAS | QAYYHVGVAA |
| | DCQSSQSVGNNAYLSWYQQK | (SEQ ID NO: 11) | (SEQ ID NO: | (SEQ ID NO: 35) |
| | PGQPPKLLIYYASTLASGVPSR | | 19) | |
| | FSGSGSGTHFTLTISGVQCDDA | | | |
| | ATYYCQAYYHVGVAAFGGGT | | | |
| | EVVVK (SEQ ID NO: 48) | | | |
| 17D13 | DVVMTQTPASVSESVGGTVTI | QASQDLYSNCLS | LTSTLAS | QGFHGYGVGAA |
| | KCQASQDLYSNCLSWYQQKP | (SEQ ID NO: 12) | (SEQ ID NO: | (SEQ ID NO: 36) |
| | GQRPKLLMYLTSTLASGVPSR | | 23) | |
| | FKGSGSGTDFTLTISDLECADA | | | |
| | ATYYCQGFHGYGVGAAFGGG | | | |
| | TEVVVK (SEQ ID NO: 49) | | | |
| 18J5 | DVVMTQTPASVEAAVGGTVTI | QASQSINSWLS | AASTLAS | QGYDGSSGSAAS |
| | KCQASQSINSWLSWYQQKPG | (SEQ ID NO: 13) | (SEQ ID NO: | (SEQ ID NO: 37) |
| | QRPKLLIYAASTLASGVSSRFK | | 24) | |
| | GSKSGTEFTLTISGVQCDDAAT | | | |
| | YYCQGYDGSSGSAASFGGGTE | | | |
| | VVVK (SEQ ID NO: 50) | | | |

TABLE 1B
Exemplary Heavy Chain Variable Region Amino Acid Sequences for Anti-
Nucleic Acid and Anti-GalNAc Moiety Antibodies

| | | | | |
|---|---|---|---|---|
| Antibody | | | | |
| ID. | VH Amino Acid Sequence | CDRH1 | CDRH2 | CDRH3 |

Pan-specific mAbs

| | | | | |
|---|---|---|---|---|
| 14K10 | QSLEESGGDLVKPGASLTLTC | SSYYMW (SEQ ID | CINGGSRGTTYYA | DPYGFSGSIYAL |
| | TASGFSFSSSYYMWWVRQAP | NO: 51) | SWAKG (SEQ ID | (SEQ ID NO: 77) |
| | GKGLEWIACINGGSRGTTYY | | NO: 64) | |
| | ASWAKGRFTISKTSSTTVTLQ | | | |
| | MTSLTVADTATYFCARDPYG | | | |
| | FSGSIYALWGPGTLVTVSS | | | |
| | (SEQ ID NO: 90) | | | |
| 14F4 | QSLEESGGRLVTPGTPLTLSC | GHYMS (SEQ ID | HIYGSGRGLWYA | YRPVDYVMDI |
| | KASGLSLSGHYMSWVRQAPG | NO: 52) | NWAKG (SEQ ID | (SEQ ID NO: 78) |
| | KGLEWIGHIYGSGRGLWYAN | | NO: 65) | |
| | WAKGRFTISKTSTTVDLKIISP | | | |
| | TNEDTATYFCARYRPVDYVM | | | |
| | DIWGPGTLVTVSL (SEQ ID | | | |
| | NO: 91) | | | |
| 5I17 | QEQLEESGGDLVKPEGSLTLT | SNYYMC (SEQ ID | CIYAGSSGSTYYA | ERENYIGVGYYL |
| | CTASGFSFSSNYYMCWVRQA | NO: 53) | SWAKG (SEQ ID | (SEQ ID NO: 79) |
| | PGKGLEWIACIYAGSSGSTYY | | NO: 66) | |
| | ASWAKGRFTVSKTSSTTVTL | | | |
| | QMTSLTAADTATYFCARERE | | | |
| | NYIGVGYYLWGPGTLVTVSS | | | |
| | (SEQ ID NO: 92) | | | |

1851 RNAi construct-specific mAbs

| | | | | |
|---|---|---|---|---|
| 17K13 | EQLKESGGRLVTPGTPLTLTC | GSYWIN (SEQ ID | IIAAGGRIWYASSV | DDIGIPGGDI |
| | TVSGFSLSGSYWINWVRQAP | NO: 54) | KG (SEQ ID NO: 67) | (SEQ ID NO: 80) |
| | GKGLEWIAIIAAGGRIWYASS | | | |
| | VKGRFTISKTSTTVDLKMTSP | | | |
| | TTEDTATYFCARDDIGIPGGDI | | | |
| | WGPGTLVTVSL (SEQ ID NO: | | | |
| | 93) | | | |
| 17F22 | QEQLKESGGGLVTPGGTLTLT | SYFMS (SEQ ID | IIYGRDKTYYATW | SSSSGRGLYYGG |
| | CTASGFTINSYFMSWVRQAP | NO: 55) | TKG (SEQ ID NO: | MDP (SEQ ID NO: |
| | GKGLEWIGIIYGRDKTYYAT | | 68) | 81) |
| | WTKGRFTISKTSTTVDLIITSP | | | |
| | TTEDTATYFCARSSSSGRGLY | | | |
| | YGGMDPWGPGTLVTVSS | | | |
| | (SEQ ID NO: 94) | | | |
| 20K24 | QSLEESGGDLVKPGASLTLTC | SGYYMC (SEQ ID | CIYAGSGARTYYA | DPYSVNDPVGD |
| | TASGFSFSSGYYMCWVRQAP | NO: 56) | SWAKG (SEQ ID | TL (SEQ ID NO: |
| | GKGLEWIACIYAGSGARTYY | | NO: 69) | 82) |
| | ASWAKGRFTISKTSSTTVTLQ | | | |
| | MTSLTAADTATYFCARDPYS | | | |
| | VNDPVGDTLWGPGTLVTVSS | | | |
| | (SEQ ID NO: 95) | | | |
| 20P19 | ERLEESGGDLVKPGASLTLTC | NNYIMC (SEQ ID | CISTLTTATYYAT | DGWSGDGVITF |
| | KASGADFNNNYIMCWVRQA | NO: 57) | WAKG (SEQ ID | NL (SEQ ID NO: |
| | PGKGLEWIACISTLTTATYYA | | NO: 70) | 83) |
| | TWAKGRFTISTTSSTTVTLQM | | | |
| | TSLTAADTATYFCAGDGWSG | | | |
| | DGVITFNLWGPGTLVTVSS | | | |
| | (SEQ ID NO: 96) | | | |
| 19F24 | QSLEESGGRLVTPGTPLTLTC | SSYWIN (SEQ ID | IMPAGGRPYYAT | DDIGTPGGDI |
| | TASGFSLSSSYWINWVRQAP | NO: 58) | WAKG (SEQ ID | (SEQ ID NO: 84) |
| | GKGLEWIAIMPAGGRPYYAT | | NO: 71) | |
| | WAKGRFIISKTSTTVDLKMTS | | | |
| | PTTEDTATYFCARDDIGTPGG | | | |
| | DIWGPGTLVTVSL (SEQ ID | | | |
| | NO: 97) | | | |

GalNAc moiety-specific mAbs

| | | | | |
|---|---|---|---|---|
| 14D4 | QSVEESGGRLVTPGTPLTLTC | RYVVD (SEQ ID | TIGYGSTWYASW | GNVGSTGVSI |
| | TVSRIDLSRYVVDWVRQAPG | NO: 59) | VKG (SEQ ID NO: | (SEQ ID NO: 85) |
| | EGLEWIGTIGYGSTWYASWV | | 72) | |
| | KGRFTISRTSTTVDLKMTSLT | | | |
| | TEDTATYFCARGNVGSTGVSI | | | |
| | WGPGTLVTVSL (SEQ ID NO: | | | |
| | 98) | | | |
| 16I3 | QSVEESGGRLVAPGTPLTLTC | YFGMY (SEQ ID | TIDSSDIIYYASWA | SGGVAGGDSV |
| | TVSGFSLSYFGMYWVRQAPG | NO: 60) | KG (SEQ ID NO: | (SEQ ID NO: 86) |
| | RGLEWIGTIDSSDIIYYASWA | | 73) | |
| | KGRFTISKTSTTVDLKITSPTT | | | |
| | EDTATYFCARSGGVAGGDSV | | | |
| | WGPGTLVTVSL (SEQ ID NO: | | | |
| | 99) | | | |
| 16A22 | QSLEESGGRLVTPGGYLTLTC | SYDMG (SEQ ID | YIFINDNTYYATW | GYFGGMDP |
| | TVSGFSLSSYDMGWVRQAPG | NO: 61) | AKG (SEQ ID NO: | (SEQ ID NO: 87) |
| | KGLEWIGYIFINDNTYYATW | | 74) | |
| | AKGRFTISKTSTTMDLKMTSL | | | |
| | TTEDTATYFCVRGYFGGMDP | | | |
| | WGPGTLVTVSS (SEQ ID NO: | | | |
| | 100) | | | |
| 17D13 | QSVEESGGRLVTPGASLTLTC | TNAMT (SEQ ID | YIYEGSGNTFYAS | GYLGAMDP |
| | TVSGIDFSTNAMTWVRQAPG | NO: 62) | WAKG (SEQ ID | (SEQ ID NO: 88) |
| | KGLEWIGYIYEGSGNTFYAS | | NO: 75) | |
| | WAKGRFTISRTSTTVDLKMTS | | | |
| | LTMEDTATYFCARGYLGAM | | | |
| | DPWGPGTLVTVSS (SEQ ID | | | |
| | NO: 101) | | | |
| 18J5 | QSVEESGGGLVKPGASLTLTC | DHYMS (SEQ ID | YISEGGATYYASW | GWLAAFDP |
| | QVSGFSLSDHYMSWVRQAPG | NO: 63) | AKG (SEQ ID NO: | (SEQ ID NO: 89) |
| | KGLEWVAYISEGGATYYASW | | 76) | |
| | AKGRFTISKTSSTTVDLKMTS | | | |
| | LTTEDTATYFCARGWLAAFD | | | |
| | PWGPGTLVTVSS (SEQ ID | | | |
| | NO: 102) | | | |

The antibodies or antigen-binding fragments of the invention may comprise one or more of the light chain CDRs (i.e. CDRLs) and/or heavy chain CDRs (i.e. CDRHs) presented in Tables 1A and 1B. For instance, in some embodiments, the antibodies or antigen-binding fragments of the invention that specifically bind to a chemically-modified nucleic acid molecule independent of nucleotide sequence (e.g. a pan-specific antibody or antigen-binding fragment thereof) comprise a CDRL1 comprising a sequence selected from SEQ ID NOs: 1 to 3; a CDRL2 comprising a sequence selected from SEQ ID NOs: 14 to 16; a CDRL3 comprising a sequence selected from SEQ ID NOs: 25 to 27; a CDRH1 comprising a sequence selected from SEQ ID NOs: 51 to 53; a CDRH2 comprising a sequence selected from SEQ ID NOs: 64 to 66; and a CDRH3 comprising a sequence selected from SEQ ID NOs: 77 to 79. In one embodiment, the pan-specific antibody or antigen-binding fragment thereof of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 1, 14, and 25, respectively, and CDRH1, CDRH2, and CDRH3 have the sequence of SEQ ID NOs: 51, 64, and 77, respectively. In another embodiment, the pan-specific antibody or antigen-binding fragment thereof of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 2, 15, and 26, respectively, and CDRH1, CDRH2, and CDRH3 have the sequence of SEQ ID NOs: 52, 65, and 78, respectively. In yet another embodiment, the pan-specific antibody or antigen-binding fragment thereof of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 3, 16, and 27, respectively, and CDRH1, CDRH2, and CDRH3 have the sequence of SEQ ID NOs: 53, 66, and 79, respectively.

In certain embodiments, the antibodies or antigen-binding fragments of the invention that bind in a sequence specific manner to an RNAi construct comprising the nucleotide sequence of SEQ ID NO: 192 (e.g. an 1851 RNAi construct-specific antibody or antigen-binding fragment thereof) comprise a CDRL1 comprising a sequence selected from SEQ ID NOs: 4 to 8; a CDRL2 comprising a sequence selected from SEQ ID NOs: 17 to 20; a CDRL3 comprising a sequence selected from SEQ ID NOs: 28 to 32; a CDRH1 comprising a sequence selected from SEQ ID NOs: 54 to 58; a CDRH2 comprising a sequence selected from SEQ ID NOs: 67 to 71; and a CDRH3 comprising a sequence selected from SEQ ID NOs: 80 to 84. In one embodiment, the 1851 RNAi construct-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 4, 17, and 28, respectively, and CDRH1, CDRH2, and CDRH3 have the sequence of SEQ ID NOs: 54, 67, and 80, respectively. In another embodiment, the 1851 RNAi construct-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 5, 18, and 29, respectively, and CDRH1, CDRH2, and CDRH3 have the sequence of SEQ ID NOs: 55, 68, and 81, respectively. In another embodiment, the 1851 RNAi construct-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 6, 19, and 30, respectively, and CDRH1, CDRH2, and CDRH3 have the sequence of SEQ ID NOs: 56, 69, and 82, respectively. In yet another embodiment, the 1851 RNAi construct-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 7, 20, and 31, respectively, and CDRH1, CDRH2, and CDRH3 have the sequence of SEQ ID NOs: 57, 70, and 83, respectively. In still another embodiment, the 1851 RNAi construct-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 8, 17, and 32, respectively, and CDRH1, CDRH2, and CDRH3 have the sequence of SEQ ID NOs: 58, 71, and 84, respectively.

In some embodiments, the antibodies or antigen-binding fragments of the invention that specifically bind to a GalNAc moiety (e.g. a GalNAc moiety-specific antibody or antigen-binding fragment thereof) comprise a CDRL1 comprising a sequence selected from SEQ ID NOs: 9 to 13; a CDRL2 comprising a sequence selected from SEQ ID NOs: 19 and 21 to 24; a CDRL3 comprising a sequence selected from SEQ ID NOs: 33 to 37; a CDRH1 comprising a sequence selected from SEQ ID NOs: 59 to 63; a CDRH2 comprising a sequence selected from SEQ ID NOs: 72 to 76; and a CDRH3 comprising a sequence selected from SEQ ID NOs: 85 to 89. In one embodiment, the GalNAc moiety-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 9, 21, and 33, respectively, and CDRH1, CDRH2, and CDRH3 have the sequence of SEQ ID NOs: 59, 72, and 85, respectively. In another embodiment, the GalNAc moiety-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 10, 22, and 34, respectively, and CDRH1, CDRH2, and CDRH3 have the sequence of SEQ ID NOs: 60, 73, and 86, respectively. In another embodiment, the GalNAc moiety-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 11, 19, and 35, respectively, and CDRH1, CDRH2, and CDRH3 have the sequence of SEQ ID NOs: 61, 74, and 87, respectively. In yet another embodiment, the GalNAc moiety-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 12, 23, and 36, respectively, and CDRH1, CDRH2, and CDRH3 have the sequence of SEQ ID NOs: 62, 75, and 88, respectively. In still another embodiment, the GalNAc moiety-specific antibody or antigen-binding fragment of the invention comprises a light chain variable region comprising a CDRL1, a CDRL2, and a CDRL3, and a heavy chain variable region comprising a CDRH1, a CDRH2, and a CDRH3, wherein CDRL1, CDRL2, and CDRL3 have the sequence of SEQ ID NOs: 13, 24, and 37, respectively, and CDRH1, CDRH2, and CDRH3 have the sequence of SEQ ID NOs: 63, 76, and 89, respectively.

In some embodiments, the antibodies or antigen-binding fragments of the invention comprise an immunoglobulin heavy chain variable region (VH) and an immunoglobulin light chain variable region (VL) from one of the monoclonal antibodies described herein. The “variable region,” used interchangeably herein with “variable domain” (variable region of a light chain (VL), variable region of a heavy chain (VH)), refers to the region in each of the light and heavy immunoglobulin chains which is involved directly in binding the antibody to the antigen. As discussed above, the regions of variable light and heavy chains have the same general structure and each region comprises four framework (FR) regions, the sequences of which are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site. Thus, in some embodiments, the antibodies and antigen-binding fragments of the invention may comprise a light chain variable region presented in Table 1A, and/or a heavy chain variable region presented in Table 1B, or variants of these light chain and heavy chain variable regions.

In certain embodiments, the pan-specific antibodies or antigen-binding fragments of the invention comprise a light chain variable region comprising a sequence selected from SEQ ID NOs: 38-40 or a sequence that is at least 90% identical or at least 95% identical to a sequence selected from SEQ ID NOs: 38-40. In these and other embodiments, the pan-specific antibodies or antigen-binding fragments of the invention comprise a heavy chain variable region comprising a sequence selected from SEQ ID NOs: 90-92 or a sequence that is at least 90% identical or at least 95% identical to a sequence selected from SEQ ID NOs: 90-92. In one embodiment, the pan-specific antibody or antigen-binding fragment of the invention comprises a light chain variable region comprising the sequence of SEQ ID NO: 38 and a heavy chain variable region comprising the sequence of SEQ ID NO: 90. In another embodiment, the pan-specific antibody or antigen-binding fragment of the invention comprises a light chain variable region comprising the sequence of SEQ ID NO: 39 and a heavy chain variable region comprising the sequence of SEQ ID NO: 91. In another embodiment, the pan-specific antibody or antigen-binding fragment of the invention comprises a light chain variable region comprising the sequence of SEQ ID NO: 40 and a heavy chain variable region comprising the sequence of SEQ ID NO: 92.

In other embodiments, the 1851 RNAi construct-specific antibodies or antigen-binding fragments of the invention comprise a light chain variable region comprising a sequence selected from SEQ ID NOs: 41-45 or a sequence that is at least 90% identical or at least 95% identical to a sequence selected from SEQ ID NOs: 41-45. In these and other embodiments, the 1851 RNAi construct-specific antibodies or antigen-binding fragments of the invention comprise a heavy chain variable region comprising a sequence selected from SEQ ID NOs: 93-97 or a sequence that is at least 90% identical or at least 95% identical to a sequence selected from SEQ ID NOs: 93-97. In one embodiment, the 1851 RNAi construct-specific antibody or antigen-binding fragment of the invention comprises a light chain variable region comprising the sequence of SEQ ID NO: 41 and a heavy chain variable region comprising the sequence of SEQ ID NO: 93. In another embodiment, the 1851 RNAi construct-specific antibody or antigen-binding fragment of the invention comprises a light chain variable region comprising the sequence of SEQ ID NO: 42 and a heavy chain variable region comprising the sequence of SEQ ID NO: 94. In another embodiment, the 1851 RNAi construct-specific antibody or antigen-binding fragment of the invention comprises a light chain variable region comprising the sequence of SEQ ID NO: 43 and a heavy chain variable region comprising the sequence of SEQ ID NO: 95. In yet another embodiment, the 1851 RNAi construct-specific antibody or antigen-binding fragment of the invention comprises a light chain variable region comprising the sequence of SEQ ID NO: 44 and a heavy chain variable region comprising the sequence of SEQ ID NO: 96. In still another embodiment, the 1851 RNAi construct-specific antibody or antigen-binding fragment of the invention comprises a light chain variable region comprising the sequence of SEQ ID NO: 45 and a heavy chain variable region comprising the sequence of SEQ ID NO: 97.

In some embodiments, the GalNAc moiety-specific antibodies or antigen-binding fragments of the invention comprise a light chain variable region comprising a sequence selected from SEQ ID NOs: 46-50 or a sequence that is at least 90% identical or at least 95% identical to a sequence selected from SEQ ID NOs: 46-50. In these and other embodiments, the GalNAc moiety-specific antibodies or antigen-binding fragments of the invention comprise a heavy chain variable region comprising a sequence selected from SEQ ID NOs: 98-102 or a sequence that is at least 90% identical or at least 95% identical to a sequence selected from SEQ ID NOs: 98-102. In one embodiment, the GalNAc moiety-specific antibody or antigen-binding fragment of the invention comprises a light chain variable region comprising the sequence of SEQ ID NO: 46 and a heavy chain variable region comprising the sequence of SEQ ID NO: 98. In another embodiment, the GalNAc moiety-specific antibody or antigen-binding fragment of the invention comprises a light chain variable region comprising the sequence of SEQ ID NO: 47 and a heavy chain variable region comprising the sequence of SEQ ID NO: 99. In another embodiment, the GalNAc moiety-specific antibody or antigen-binding fragment of the invention comprises a light chain variable region comprising the sequence of SEQ ID NO: 48 and a heavy chain variable region comprising the sequence of SEQ ID NO: 100. In yet another embodiment, the GalNAc moiety-specific antibody or antigen-binding fragment of the invention comprises a light chain variable region comprising the sequence of SEQ ID NO: 49 and a heavy chain variable region comprising the sequence of SEQ ID NO: 101. In still another embodiment, the GalNAc moiety-specific antibody or antigen-binding fragment of the invention comprises a light chain variable region comprising the sequence of SEQ ID NO: 50 and a heavy chain variable region comprising the sequence of SEQ ID NO: 102.

The term “identity,” as used herein, refers to a relationship between the sequences of two or more polypeptide molecules or two or more nucleic acid molecules, as determined by aligning and comparing the sequences. “Percent identity,” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an algorithm). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, WI). The computer algorithm GAP is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:
Algorithm: Needleman et al. 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The antibodies of the invention can comprise any immunoglobulin constant region. The term “constant region,” used interchangeably herein with “constant domain” refers to all domains of an antibody other than the variable region. The constant region is not involved directly in binding of an antigen but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. Antibodies provided herein that comprise constant regions from one subclass or from one species (e.g. rabbit) can be changed to antibodies comprising constant regions from a different subclass or different species (e.g. mouse or human) using recombinant DNA techniques. Thus, antibodies comprising rabbit IgG constant regions can be converted to antibodies comprising IgG constant regions from a different species, such as mouse or human, depending on the desired application for the antibodies. Immunoglobulin constant regions from various subclasses of antibodies from several species are known in the art and can be combined with the variable region sequences provided in Tables 1A and 1B to form complete antibody light and heavy chains with the desired isotype. Further, each of the so generated heavy and light chain sequences may be combined to form a complete antibody structure (e.g. comprising two light chains and two heavy chains).

Full-length light chain and full-length heavy chain amino acid sequences of exemplary monoclonal antibodies of the invention, including pan-specific antibodies, 1851 RNAi construct-specific antibodies, and GalNAc moiety-specific antibodies, are set forth below in Table 2.

TABLE 2
Exemplary Light Chain and Heavy Chain Amino Acid Sequences for Anti-Nucleic
Acid and Anti-GalNAc Moiety Antibodies

| | | |
|---|---|---|
| Antibody | | |
| ID. | Light Chain Amino Acid Sequence | Heavy Chain Amino Acid Sequence |

Pan-specific mAbs

| | | |
|---|---|---|
| 14K10 | ALVMTQTPSPVSAAVGGTVTINCQASQS | QSLEESGGDLVKPGASLTLTCTASGFSFSSSYYM |
| | VLNNNYLSWYQQKPGQPPKLLIYSASKL | WWVRQAPGKGLEWIACINGGSRGTTYYASWA |
| | ATGVPSRFSGSGSGTQFTLTISGVQCDDA | KGRFTISKTSSTTVTLQMTSLTVADTATYFCAR |
| | ATYYCAGYKNFGNDDNAFGGGTEVVV | DPYGFSGSIYALWGPGTLVTVSSGQPKAPSVFPL |
| | KGDPVAPTVLLFPPSSDEVATGTVTIVCV | APCCGDTPSSTVTLGCLVKGYLPEPVTVTWNSG |
| | ANKYFPDVTVTWEVDGTTQTTGIENSKT | TLTNGVRTFPSVRQSSGLYSLSSVVSVTSSSQPV |
| | PQNSADCTYNLSSTLTLTSTQYNSHKEY | TCNVAHPATNTKVDKTVAPSTCSKPTCPPPELL |
| | TCKVTQGTTSVVQSFSRKNC (SEQ ID | GGPSVFIFPPKPKDTLMISRTPEVTCVVVDVSQD |
| | NO: 103) | DPEVQFTWYINNEQVRTARPPLREQQFNSTIRV |
| | | VSTLPIAHQDWLRGKEFKCKVHNKALPAPIEKTI |
| | | SKARGQPLEPKVYTMGPPREELSSRSVSLTCMIN |
| | | GFYPSDISVEWEKNGKAEDNYKTTPAVLDSDGS |
| | | YFLYSKLSVPTSEWQRGDVFTCSVMHEALHNH |
| | | YTQKSISRSPGK (SEQ ID NO: 116) |
| 14F4 | AQVLTQTSSPVSVNMGGTVTINCQSSLS | QSLEESGGRLVTPGTPLTLSCKASGLSLSGHYMS |
| | VNRSDLSWYQQKPGQPPKLLIYLASNLE | WVRQAPGKGLEWIGHIYGSGRGLWYANWAKG |
| | SGVPSRFKGSGSGTHFTLTINGVECDDA | RFTISKTSTTVDLKIISPTNEDTATYFCARYRPVD |
| | ATYYCAGGYSSGNDRNAFGGGTEVVVK | YVMDIWGPGTLVTVSLGQPKAPSVFPLAPCCGD |
| | GDPVAPTVLLFPPSSDEVATGTVTIVCVA | TPSSTVTLGCLVKGYLPEPVTVTWNSGTLTNGV |
| | NKYFPDVTVTWEVDGTTQTTGIENSKTP | RTFPSVRQSSGLYSLSSVVSVTSSSQPVTCNVAH |
| | QNSADCTYNLSSTLTLTSTQYNSHKEYT | PATNTKVDKTVAPSTCSKPTCPPPELLGGPSVFIF |
| | CKVTQGTTSVVQSFSRKNC (SEQ ID NO: | PPKPKDTLMISRTPEVTCVVVDVSQDDPEVQFT |
| | 104) | WYINNEQVRTARPPLREQQFNSTIRVVSTLPIAH |
| | | QDWLRGKEFKCKVHNKALPAPIEKTISKARGQP |
| | | LEPKVYTMGPPREELSSRSVSLTCMINGFYPSDI |
| | | SVEWEKNGKAEDNYKTTPAVLDSDGSYFLYSK |
| | | LSVPTSEWQRGDVFTCSVMHEALHNHYTQKSIS |
| | | RSPGK (SEQ ID NO: 117) |
| 5I17 | ALVMTQTPSPVSAAVGGTVTISCQASQS | QEQLEESGGDLVKPEGSLTLTCTASGFSFSSNYY |
| | VANNNYLAWYQQKPGQPPKLLIYQAST | MCWVRQAPGKGLEWIACIYAGSSGSTYYASWA |
| | LASGVPSRFKGSGSGTQFTLTISGVECDD | KGRFTVSKTSSTTVTLQMTSLTAADTATYFCAR |
| | AATYYCAGYRSYINADNAFGGGTEVVV | ERENYIGVGYYLWGPGTLVTVSSGQPKAPSVFP |
| | EGDPVAPTVLLFPPSSDEVATGTVTIVCV | LAPCCGDTPSSTVTLGCLVKGYLPEPVTVTWNS |
| | ANKYFPDVTVTWEVDGTTQTTGIENSKT | GTLTNGVRTFPSVRQSSGLYSLSSVVSVTSSSQP |
| | PQNSADCTYNLSSTLTLTSTQYNSHKEY | VTCNVAHPATNTKVDKTVAPSTCSKPTCPPPEL |
| | TCKVTQGTTSVVQSFSRKNC (SEQ ID | LGGPSVFIFPPKPKDTLMISRTPEVTCVVVDVSQ |
| | NO: 105) | DDPEVQFTWYINNEQVRTARPPLREQQFNSTIR |
| | | VVSTLPIAHQDWLRGKEFKCKVHNKALPAPIEK |
| | | TISKARGQPLEPKVYTMGPPREELSSRSVSLTCM |
| | | INGFYPSDISVEWEKNGKAEDNYKTTPAVLDSD |
| | | GSYFLYSKLSVPTSEWQRGDVFTCSVMHEALH |
| | | NHYTQKSISRSPGK (SEQ ID NO: 118) |

1851 RNAi construct-specific mAbs

| | | |
|---|---|---|
| 17K13 | AQVLTQTPSSVSAAVGGTVTINCQSSQS | EQLKESGGRLVTPGTPLTLTCTVSGFSLSGSYWI |
| | VWQKNWLSWFQQKPGQPPKLLIYRAST | NWVRQAPGKGLEWIAIIAAGGRIWYASSVKGRF |
| | LASGVPSRFKSSGSGTQFTLTISGVQCDD | TISKTSTTVDLKMTSPTTEDTATYFCARDDIGIP |
| | AATYYCAGAYSSNSDVRAFGGGTELVV | GGDIWGPGTLVTVSLGQPKAPSVFPLAPCCGDT |
| | KGDPVAPTVLLFPPSSDEVATGTVTIVCV | PSSTVTLGCLVKGYLPEPVTVTWNSGTLTNGVR |
| | ANKYFPDVTVTWEVDGTTQTTGIENSKT | TFPSVRQSSGLYSLSSVVSVTSSSQPVTCNVAHP |
| | PQNSADCTYNLSSTLTLTSTQYNSHKEY | ATNTKVDKTVAPSTCSKPTCPPPELLGGPSVFIFP |
| | TCKVTQGTTSVVQSFSRKNC (SEQ ID | PKPKDTLMISRTPEVTCVVVDVSQDDPEVQFTW |
| | NO: 106) | YINNEQVRTARPPLREQQFNSTIR VVSTLPIAHQ |
| | | DWLRGKEFKCKVHNKALPAPIEKTISKARGQPL |
| | | EPKVYTMGPPREELSSRSVSLTCMINGFYPSDIS |
| | | VEWEKNGKAEDNYKTTPAVLDSDGSYFLYSKL |
| | | SVPTSEWQRGDVFTCSVMHEALHNHYTQKSISR |
| | | SPGK (SEQ ID NO: 119) |
| 17F22 | AQVLTQTASPVSAAVGSTVTINCQASQS | QEQLKESGGGLVTPGGTLTLTCTASGFTINSYF |
| | VYNNNNLAWYQQKPGQPPKLLIYYTST | MSWVRQAPGKGLEWIGIIYGRDKTYYATWTKG |
| | LASGVSSRFKGSGSGTQFTLTISGMQCD | RFTISKTSTTVDLIITSPTTEDTATYFCARSSSSGR |
| | DAATYYCQGEFACSTADCLTFGGGTEV | GLYYGGMDPWGPGTLVTVSSGQPKAPSVFPLA |
| | VVKGDPVAPTVLLFPPSSDEVATGTVTIV | PCCGDTPSSTVTLGCLVKGYLPEPVTVTWNSGT |
| | CVANKYFPDVTVTWEVDGTTQTTGIENS | LTNGVRTFPSVRQSSGLYSLSSVVSVTSSSQPVT |
| | KTPQNSADCTYNLSSTLTLTSTQYNSHK | CNVAHPATNTKVDKTVAPSTCSKPTCPPPELLG |
| | EYTCKVTQGTTSVVQSFSRKNC (SEQ ID | GPSVFIFPPKPKDTLMISRTPEVTCVVVDVSQDD |
| | NO: 107) | PEVQFTWYINNEQVRTARPPLREQQFNSTIRVVS |
| | | TLPIAHQDWLRGKEFKCKVHNKALPAPIEKTISK |
| | | ARGQPLEPKVYTMGPPREELSSRSVSLTCMINGF |
| | | YPSDISVEWEKNGKAEDNYKTTPAVLDSDGSYF |
| | | LYSKLSVPTSEWQRGDVFTCSVMHEALHNHYT |
| | | QKSISRSPGK (SEQ ID NO: 120) |
| 20K24 | ALVMTQTPASVSAAVGGTVTINCQASES | QSLEESGGDLVKPGASLTLTCTASGFSFSSGYY |
| | ISSWLAWYQQKPGQPPKLLIYYASTLAS | MCWVRQAPGKGLEWIACIYAGSGARTYYASW |
| | GVSSRFKGSGSRTEYTLTISDLECADAAT | AKGRFTISKTSSTTVTLQMTSLTAADTATYFCA |
| | YYCAGYGSANDDKNGFGGGTEVVVKG | RDPYSVNDPVGDTLWGPGTLVTVSSGQPKAPS |
| | DPVAPTVLLFPPSSDEVATGTVTIVCVAN | VFPLAPCCGDTPSSTVTLGCLVKGYLPEPVTVT |
| | KYFPDVTVTWEVDGTTQTTGIENSKTPQ | WNSGTLTNGVRTFPSVRQSSGLYSLSSVVSVTSS |
| | NSADCTYNLSSTLTLTSTQYNSHKEYTC | SQPVTCNVAHPATNTKVDKTVAPSTCSKPTCPP |
| | KVTQGTTSVVQSFSRKNC (SEQ ID NO: | PELLGGPSVFIFPPKPKDTLMISRTPEVTCVVVD |
| | 108 | VSQDDPEVQFTWYINNEQVRTARPPLREQQFNS |
| | | TIRVVSTLPIAHQDWLRGKEFKCKVHNKALPAP |
| | | IEKTISKARGQPLEPK VYTMGPPREELSSRSVSLT |
| | | CMINGFYPSDISVEWEKNGKAEDNYKTTPAVLD |
| | | SDGSYFLYSKLSVPTSEWQRGDVFTCSVMHEAL |
| | | HNHYTQKSISRSPGK (SEQ ID NO: 121) |
| 20P19 | AYDMTQTPASVEVAVGGTVTIKCQASQ | ERLEESGGDLVKPGASLTLTCKASGADFNNNYI |
| | SINNELA WYQQKPGQPPKLLIYLAFTLAS | MCWVRQAPGKGLEWIACISTLTTATYYATWAK |
| | GVPSRFKGSRSGTEFTLTISDLECADAAT | GRFTISTTSSTTVTLQMTSLTAADTATYFCAGDG |
| | YYCQQGYIISGVDNVFGGGTEVVVKGDP | WSGDGVITFNLWGPGTLVTVSSGQPKAPSVFPL |
| | VAPTVLLFPPSSDEVATGTVTIVCVANK | APCCGDTPSSTVTLGCLVKGYLPEPVTVTWNSG |
| | YFPDVTVTWEVDGTTQTTGIENSKTPQN | TLTNGVRTFPSVRQSSGLYSLSSVVSVTSSSQPV |
| | SADCTYNLSSTLTLTSTQYNSHKEYTCK | TCNVAHPATNTKVDKTVAPSTCSKPTCPPPELL |
| | VTQGTTSVVQSFSRKNC (SEQ ID NO: | GGPSVFIFPPKPKDTLMISRTPEVTCVVVDVSQD |
| | 109) | DPEVQFTWYINNEQVRTARPPLREQQFNSTIRV |
| | | VSTLPIAHQDWLRGKEFKCKVHNKALPAPIEKTI |
| | | SKARGQPLEPKVYTMGPPREELSSRSVSLTCMIN |
| | | GFYPSDISVEWEKNGKAEDNYKTTPAVLDSDGS |
| | | YFLYSKLSVPTSEWQRGDVFTCSVMHEALHNH |
| | | YTQKSISRSPGK (SEQ ID NO: 122) |
| 19F24 | AQVLTQTPSSVSAAVGGTVTINCQSSQS | QSLEESGGRLVTPGTPLTLTCTASGFSLSSSYWI |
| | VWEKNWLSWFQQKPGQPPKLLIYRAST | NWVRQAPGKGLEWIAIMPAGGRPYYATWAKG |
| | LASGVPSRFKSSGSGTQFTLTISGVQCDD | RFIISKTSTTVDLKMTSPTTEDTATYFCARDDIGT |
| | AAVYYCAGAYSVNSDVRAFGGGTELVV | PGGDIWGPGTLVTVSLGQPKAPSVFPLAPCCGD |
| | KGDPVAPTVLLFPPSSDEVATGTVTIVCV | TPSSTVTLGCLVKGYLPEPVTVTWNSGTLTNGV |
| | ANKYFPDVTVTWEVDGTTQTTGIENSKT | RTFPSVRQSSGLYSLSSVVSVTSSSQPVTCNVAH |
| | PQNSADCTYNLSSTLTLTSTQYNSHKEY | PATNTKVDKTVAPSTCSKPTCPPPELLGGPSVFIF |
| | TCKVTQGTTSVVQSFSRKNC (SEQ ID | PPKPKDTLMISRTPEVTCVVVDVSQDDPEVQFT |
| | NO: 110) | WYINNEQVRTARPPLREQQFNSTIR VVSTLPIAH |
| | | QDWLRGKEFKCKVHNKALPAPIEKTISKARGQP |
| | | LEPKVYTMGPPREELSSRSVSLTCMINGFYPSDI |
| | | SVEWEKNGKAEDNYKTTPAVLDSDGSYFLYSK |
| | | LSVPTSEWQRGDVFTCSVMHEALHNHYTQKSIS |
| | | RSPGK (SEQ ID NO: 123) |

GalNAc moiety-specific mAbs

| | | |
|---|---|---|
| 14D4 | AIVMTQTPSSKSVPVGGTVTINCQSSESV | QSVEESGGRLVTPGTPLTLTCTVSRIDLSRYVVD |
| | YENNDLSWYQQKPGQPPKLLIYWASSL | WVRQAPGEGLEWIGTIGYGSTWYASWVKGRFT |
| | ASGVPSRFEGSGSGTQFTLTISNVVCDDA | ISRTSTTVDLKMTSLTTEDTATYFCARGNVGST |
| | ATYYCAGYKSMSTDGFAFGGGTEVVVK | GVSIWGPGTLVTVSLGQPKAPSVFPLAPCCGDT |
| | GDPVAPTVLLFPPSSDEVATGTVTIVCVA | PSSTVTLGCLVKGYLPEPVTVTWNSGTLTNGVR |
| | NKYFPDVTVTWEVDGTTQTTGIENSKTP | TFPSVRQSSGLYSLSSVVSVTSSSQPVTCNVAHP |
| | QNSADCTYNLSSTLTLTSTQYNSHKEYT | ATNTKVDKTVAPSTCSKPTCPPPELLGGPSVFIFP |
| | CKVTQGTTSVVQSFSRKNC (SEQ ID NO: | PKPKDTLMISRTPEVTCVVVDVSQDDPEVQFTW |
| | 111) | YINNEQVRTARPPLREQQFNSTIRVVSTLPIAHQ |
| | | DWLRGKEFKCKVHNKALPAPIEKTISKARGQPL |
| | | EPKVYTMGPPREELSSRSVSLTCMINGFYPSDIS |
| | | VEWEKNGKAEDNYKTTPAVLDSDGSYFLYSKL |
| | | SVPTSEWQRGDVFTCSVMHEALHNHYTQKSISR |
| | | SPGK (SEQ ID NO: 124) |
| 16I3 | AIVMTQTPSSKSVPVGGTVTISCQASQSL | QSVEESGGRLVAPGTPLTLTCTVSGFSLSYFGM |
| | YKNTDLAWFQQKPGQPPKLLIYFASNLA | YWVRQAPGRGLEWIGTIDSSDIIYYASWAKGRF |
| | SGVTSRFKGSGSGTQFTLTISDVVCDDA | TISKTSTTVDLKITSPTTEDTATYFCARSGGVAG |
| | ATYYCAGYKSSTTDGFGFGGGTEVVVK | GDSVWGPGTLVTVSLGQPKAPSVFPLAPCCGDT |
| | GDPVAPTVLLFPPSSDEVATGTVTIVCVA | PSSTVTLGCLVKGYLPEPVTVTWNSGTLTNGVR |
| | NKYFPDVTVTWEVDGTTQTTGIENSKTP | TFPSVRQSSGLYSLSSVVSVTSSSQPVTCNVAHP |
| | QNSADCTYNLSSTLTLTSTQYNSHKEYT | ATNTKVDKTVAPSTCSKPTCPPPELLGGPSVFIFP |
| | CKVTQGTTSVVQSFSRKNC (SEQ ID NO: | PKPKDTLMISRTPEVTCVVVDVSQDDPEVQFTW |
| | 112) | YINNEQVRTARPPLREQQFNSTIR VVSTLPIAHQ |
| | | DWLRGKEFKCKVHNKALPAPIEKTISKARGQPL |
| | | EPKVYTMGPPREELSSRSVSLTCMINGFYPSDIS |
| | | VEWEKNGKAEDNYKTTPAVLDSDGSYFLYSKL |
| | | SVPTSEWQRGDVFTCSVMHEALHNHYTQKSISR |
| | | SPGK (SEQ ID NO: 125) |
| 16A22 | AQVLAQTPSSVSAAVGGTVTIDCQSSQS | QSLEESGGRLVTPGGYLTLTCTVSGFSLSSYDM |
| | VGNNAYLSWYQQKPGQPPKLLIYYASTL | GWVRQAPGKGLEWIGYIFINDNTYYATWAKGR |
| | ASGVPSRFSGSGSGTHFTLTISGVQCDDA | FTISKTSTTMDLKMTSLTTEDTATYFCVRGYFG |
| | ATYYCQAYYHVGVAAFGGGTEVVVKG | GMDPWGPGTLVTVSSGQPKAPSVFPLAPCCGDT |
| | DPVAPTVLLFPPSSDEVATGTVTIVCVAN | PSSTVTLGCLVKGYLPEPVTVTWNSGTLTNGVR |
| | KYFPDVTVTWEVDGTTQTTGIENSKTPQ | TFPSVRQSSGLYSLSSVVSVTSSSQPVTCNVAHP |
| | NSADCTYNLSSTLTLTSTQYNSHKEYTC | ATNTKVDKTVAPSTCSKPTCPPPELLGGPSVFIFP |
| | KVTQGTTSVVQSFSRKNC (SEQ ID NO: | PKPKDTLMISRTPEVTCVVVDVSQDDPEVQFTW |
| | 113) | YINNEQVRTARPPLREQQFNSTIR VVSTLPIAHQ |
| | | DWLRGKEFKCKVHNKALPAPIEKTISKARGQPL |
| | | EPKVYTMGPPREELSSRSVSLTCMINGFYPSDIS |
| | | VEWEKNGKAEDNYKTTPAVLDSDGSYFLYSKL |
| | | SVPTSEWQRGDVFTCSVMHEALHNHYTQKSISR |
| | | SPGK (SEQ ID NO: 126) |
| 17D13 | DVVMTQTPASVSESVGGTVTIKCQASQD | QSVEESGGRLVTPGASLTLTCTVSGIDFSTNAMT |
| | LYSNCLSWYQQKPGQRPKLLMYLTSTL | WVRQAPGKGLEWIGYIYEGSGNTFYASWAKGR |
| | ASGVPSRFKGSGSGTDFTLTISDLECADA | FTISRTSTTVDLKMTSLTMEDTATYFCARGYLG |
| | ATYYCQGFHGYGVGAAFGGGTEVVVK | AMDPWGPGTLVTVSSGQPKAPSVFPLAPCCGDT |
| | GDPVAPTVLLFPPSSDEVATGTVTIVCVA | PSSTVTLGCLVKGYLPEPVTVTWNSGTLTNGVR |
| | NKYFPDVTVTWEVDGTTQTTGIENSKTP | TFPSVRQSSGLYSLSSVVSVTSSSQPVTCNVAHP |
| | QNSADCTYNLSSTLTLTSTQYNSHKEYT | ATNTKVDKTVAPSTCSKPTCPPPELLGGPSVFIFP |
| | CKVTQGTTSVVQSFSRKNC (SEQ ID NO: | PKPKDTLMISRTPEVTCVVVDVSQDDPEVQFTW |
| | 114) | YINNEQVRTARPPLREQQFNSTIR VVSTLPIAHQ |
| | | DWLRGKEFKCKVHNKALPAPIEKTISKARGQPL |
| | | EPKVYTMGPPREELSSRSVSLTCMINGFYPSDIS |
| | | VEWEKNGKAEDNYKTTPAVLDSDGSYFLYSKL |
| | | SVPTSEWQRGDVFTCSVMHEALHNHYTQKSISR |
| | | SPGK (SEQ ID NO: 127) |
| 18J5 | DVVMTQTPASVEAAVGGTVTIKCQASQ | QSVEESGGGLVKPGASLTLTCQVSGFSLSDHYM |
| | SINSWLSWYQQKPGQRPKLLIYAASTLA | SWVRQAPGKGLEWVAYISEGGATYYASWAKG |
| | SGVSSRFKGSKSGTEFTLTISGVQCDDAA | RFTISKTSSTTVDLKMTSLTTEDTATYFCARGW |
| | TYYCQGYDGSSGSAASFGGGTEVVVKG | LAAFDPWGPGTLVTVSSGQPKAPSVFPLAPCCG |
| | DPVAPTVLLFPPSSDEVATGTVTIVCVAN | DTPSSTVTLGCLVKGYLPEPVTVTWNSGTLTNG |
| | KYFPDVTVTWEVDGTTQTTGIENSKTPQ | VRTFPSVRQSSGLYSLSSVVSVTSSSQPVTCNVA |
| | NSADCTYNLSSTLTLTSTQYNSHKEYTC | HPATNTKVDKTVAPSTCSKPTCPPPELLGGPSVF |
| | KVTQGTTSVVQSFSRKNC (SEQ ID NO: | IFPPKPKDTLMISRTPEVTCVVVDVSQDDPEVQF |
| | 115) | TWYINNEQVRTARPPLREQQFNSTIR VVSTLPIA |
| | | HQDWLRGKEFKCKVHNKALPAPIEKTISKARGQ |
| | | PLEPKVYTMGPPREELSSRSVSLTCMINGFYPSD |
| | | ISVEWEKNGKAEDNYKTTPAVLDSDGSYFLYSK |
| | | LSVPTSEWQRGDVFTCSVMHEALHNHYTQKSIS |
| | | RSPGK (SEQ ID NO: 128) |

In certain embodiments, the antibodies or antigen-binding fragments of the invention may comprise a light chain selected from the light chains provided in Table 2, and/or a heavy chain selected from the heavy chains provided in Table 2, or variants of these light chains and heavy chains. In some embodiments, the pan-specific antibody or antigen-binding fragment of the invention comprises: (a) a light chain comprising the sequence of SEQ ID NO: 103 and a heavy chain comprising the sequence of SEQ ID NO: 116; (b) a light chain comprising the sequence of SEQ ID NO: 104 and a heavy chain comprising the sequence of SEQ ID NO: 117; or (c) a light chain comprising the sequence of SEQ ID NO: 105 and a heavy chain comprising the sequence of SEQ ID NO: 118. In certain embodiments, the 1851 RNAi construct-specific antibody or antigen-binding fragment of the invention comprises: (a) a light chain comprising the sequence of SEQ ID NO: 106 and a heavy chain comprising the sequence of SEQ ID NO: 119; (b) a light chain comprising the sequence of SEQ ID NO: 107 and a heavy chain comprising the sequence of SEQ ID NO: 120; (c) a light chain comprising the sequence of SEQ ID NO: 108 and a heavy chain comprising the sequence of SEQ ID NO: 121; (d) a light chain comprising the sequence of SEQ ID NO: 109 and a heavy chain comprising the sequence of SEQ ID NO: 122; or (e) a light chain comprising the sequence of SEQ ID NO: 110 and a heavy chain comprising the sequence of SEQ ID NO: 123. In certain other embodiments, the GalNAc moiety-specific antibody or antigen-binding fragment of the invention comprises: (a) a light chain comprising the sequence of SEQ ID NO: 111 and a heavy chain comprising the sequence of SEQ ID NO: 124; (b) a light chain comprising the sequence of SEQ ID NO: 112 and a heavy chain comprising the sequence of SEQ ID NO: 125; (c) a light chain comprising the sequence of SEQ ID NO: 113 and a heavy chain comprising the sequence of SEQ ID NO: 126; (d) a light chain comprising the sequence of SEQ ID NO: 114 and a heavy chain comprising the sequence of SEQ ID NO: 127; or (e) a light chain comprising the sequence of SEQ ID NO: 115 and a heavy chain comprising the sequence of SEQ ID NO: 128. In any of the above-described embodiments, the antibody may be a rabbit monoclonal antibody, particularly a rabbit IgG antibody.

Variants of the antibodies disclosed herein are also contemplated. For instance, variants of the pan-specific antibodies may comprise a light chain comprising a sequence that is at least 80%, 85%, 90%, or 95% identical to a sequence selected from SEQ ID NOs: 103-105 and a heavy chain comprising a sequence that is at least 80%, 85%, 90%, or 95% identical to a sequence selected from SEQ ID NOs: 116-118. In some embodiments, the 1851 RNAi construct-specific antibodies may comprise a light chain comprising a sequence that is at least 80%, 85%, 90%, or 95% identical to a sequence selected from SEQ ID NOs: 106-110 and a heavy chain comprising a sequence that is at least 80%, 85%, 90%, or 95% identical to a sequence selected from SEQ ID NOs: 119-123. In certain embodiments, the GalNAc moiety-specific antibodies may comprise a light chain comprising a sequence that is at least 80%, 85%, 90%, or 95% identical to a sequence selected from SEQ ID NOs: 111-115 and a heavy chain comprising a sequence that is at least 80%, 85%, 90%, or 95% identical to a sequence selected from SEQ ID NOs: 124-128.

Antibodies or antigen-binding fragments of the invention can be produced using the methods for generating monoclonal antibodies of the invention described above. Antibodies or antigen-binding fragments of the invention can also be produced using recombinant expression methods known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988). Relevant amino acid sequences from an immunoglobulin or region thereof (e.g. variable region, constant region, etc.) may be determined by direct protein sequencing, and suitable encoding nucleotide sequences can be designed according to a universal codon table. Alternatively, genomic or cDNA encoding monoclonal antibodies or binding fragments thereof of the invention can be isolated and sequenced from cells producing such antibodies (e.g. clonal B-cells or hybridomas) using conventional procedures (e.g., by using oligonucleotide probes that are capable of binding specifically to genes encoding the heavy and light chains of the monoclonal antibodies).

Table 3 provides exemplary nucleic acid sequences encoding the light and heavy chain variable regions of the pan-specific, 1851 RNAi construct-specific, and GalNAc moiety-specific antibodies of the invention, and Table 4 lists exemplary nucleic acid sequences encoding the full-length light and heavy chains of the antibodies of the invention. Polynucleotides encoding the variable regions and full chains can be used to recombinantly express the antibodies described herein or variants thereof.

TABLE 3
Exemplary Variable Region Nucleic Acid Sequences for Anti-Nucleic Acid and
Anti-GalNAc Moiety Antibodies

| | | |
|---|---|---|
| Antibody | | |
| ID. | VL Nucleic Acid Sequence | VH Nucleic Acid Sequence |

Pan-specific mAbs

| | | |
|---|---|---|
| 14K10 | GCGCTTGTGATGACCCAGACTCCATC | CAGTCGCTGGAGGAGTCCGGGGGAGACCTGG |
| | CCCTGTGTCTGCAGCTGTGGGAGGCA | TCAAGCCTGGGGCATCCCTGACACTCACCTGC |
| | CAGTCACCATCAACTGCCAGGCCAGT | ACAGCCTCTGGATTCTCCTTCAGTAGCAGCTA |
| | CAGAGTGTTCTTAATAACAACTACTTA | TTACATGTGGTGGGTCCGCCAGGCTCCAGGGA |
| | TCCTGGTATCAGCAGAAACCAGGGCA | AGGGGCTGGAGTGGATCGCATGCATTAATGG |
| | GCCTCCCAAGCTCCTGATCTATTCTGC | TGGTAGTCGTGGTACCACTTACTACGCGAGCT |
| | ATCCAAACTGGCAACTGGGGTCCCAT | GGGCGAAAGGCCGATTCACCATCTCCAAAAC |
| | CGCGGTTCAGCGGCAGTGGATCTGGG | CTCGTCGACCACGGTGACTCTGCAAATGACCA |
| | ACACAGTTTACTCTCACCATCAGCGG | GTCTGACAGTCGCGGACACGGCCACCTATTTC |
| | CGTGCAGTGTGACGATGCTGCCACTT | TGTGCGAGAGATCCATATGGTTTTAGTGGTAG |
| | ACTACTGTGCAGGCTATAAAAATTTC | TATTTATGCCTTGTGGGGCCCAGGCACCCTGG |
| | GGTAATGATGATAATGCTTTCGGCGG | TCACCGTCTCCTCA (SEQ ID NO: 142) |
| | AGGGACCGAGGTGGTGGTCAAA (SEQ | |
| | ID NO: 129) | |
| 14F4 | GCGCAAGTGCTGACCCAGACATCATC | CAGTCGCTGGAGGAGTCCGGGGGTCGCCTGG |
| | CCCCGTGTCTGTAAATATGGGAGGCA | TCACGCCTGGGACACCCCTGACACTCTCCTGC |
| | CAGTCACCATCAACTGCCAGTCCAGT | AAAGCCTCTGGACTCTCCCTCAGTGGCCACTA |
| | CTGAGTGTTAATAGGAGCGACTTATC | CATGAGCTGGGTCCGCCAGGCTCCAGGGAAG |
| | CTGGTATCAGCAGAAACCAGGGCAGC | GGGCTGGAGTGGATCGGACACATTTATGGTA |
| | CTCCCAAACTCCTGATCTATCTGGCAT | GTGGCCGTGGTTTATGGTACGCGAACTGGGCG |
| | CCAATCTGGAATCTGGGGTCCCCTCG | AAAGGCCGATTCACCATCTCCAAGACCTCGAC |
| | CGGTTCAAAGGCAGTGGATCTGGGAC | CACGGTAGATCTGAAAATTATCAGTCCGACA |
| | GCACTTCACTCTCACCATCAACGGCGT | AACGAGGACACGGCCACCTATTTCTGTGCCAG |
| | GGAATGTGACGATGCTGCCACTTACT | ATATCGCCCTGTTGATTATGTGATGGACATCT |
| | ACTGTGCAGGCGGTTATAGTAGTGGT | GGGGCCCAGGCACCCTGGTCACCGTCTCGTTA |
| | AATGATAGGAATGCTTTCGGCGGAGG | (SEQ ID NO: 143) |
| | GACCGAGGTGGTGGTCAAA (SEQ ID | |
| | NO: 130) | |
| 5I17 | GCGCTTGTGATGACCCAGACTCCATC | CAGGAGCAGCTGGAGGAGTCCGGGGGAGACC |
| | CCCTGTGTCTGCAGCTGTGGGAGGCA | TGGTCAAGCCTGAGGGATCCCTGACACTCACC |
| | CAGTCACCATCAGTTGCCAGGCCAGT | TGCACAGCCTCTGGATTCTCCTTCAGTAGCAA |
| | CAGAGTGTTGCTAATAACAACTACTT | CTACTACATGTGCTGGGTCCGCCAGGCTCCAG |
| | AGCCTGGTATCAGCAGAAACCAGGGC | GGAAGGGACTGGAGTGGATCGCATGCATTTA |
| | AGCCTCCCAAGCTCCTGATTTACCAG | TGCTGGTAGTAGTGGTAGCACTTACTACGCGA |
| | GCATCCACTCTGGCATCTGGGGTCCC | GCTGGGCGAAAGGCCGGTTCACCGTCTCCAA |
| | ATCGCGGTTCAAAGGCAGTGGATCTG | AACCTCGTCGACCACGGTGACTCTGCAAATGA |
| | GGACACAGTTCACTCTCACCATCAGT | CCAGTCTGACAGCCGCGGACACGGCCACCTA |
| | GGCGTGGAGTGTGACGATGCTGCCAC | TTTCTGTGCGAGAGAGCGTGAAAATTATATTG |
| | TTACTACTGTGCAGGCTATCGAAGTTA | GTGTTGGTTACTATTTGTGGGGCCCAGGCACC |
| | TATTAATGCTGATAATGCTTTCGGCGG | CTGGTCACCGTCTCCTCA (SEQ ID NO: 144) |
| | AGGGACCGAGGTGGTGGTCGAA (SEQ | |
| | ID NO: 131) | |

1851 RNAi construct-specific mAbs

| | | |
|---|---|---|
| 17K13 | GCCCAAGTGCTGACCCAGACTCCATC | GAGCAGCTGAAGGAGTCCGGGGGTCGCCTGG |
| | CTCCGTGTCTGCAGCTGTGGGAGGCA | TCACGCCTGGGACACCCCTGACACTCACCTGC |
| | CAGTCACCATCAATTGCCAGTCCAGT | ACCGTCTCTGGCTTCTCCCTCAGTGGCAGCTA |
| | CAGAGTGTTTGGCAGAAAAACTGGTT | TTGGATAAATTGGGTTCGCCAGGCTCCAGGGA |
| | ATCCTGGTTTCAGCAGAAACCAGGGC | AGGGGCTGGAATGGATCGCGATTATTGCGGC |
| | AGCCTCCCAAGCTCCTGATCTACAGG | AGGTGGGCGTATATGGTACGCGAGCTCGGTG |
| | GCGTCCACTCTGGCATCTGGGGTCCC | AAAGGCCGATTCACCATCTCCAAAACCTCGAC |
| | ATCGCGGTTCAAGAGCAGTGGATCTG | CACGGTGGATCTGAAAATGACCAGTCCGACA |
| | GGACACAGTTCACTCTCACCATCAGC | ACCGAGGACACGGCCACCTATTTCTGTGCCAG |
| | GGCGTGCAGTGTGACGATGCTGCCAC | AGACGATATTGGTATTCCTGGTGGGGACATCT |
| | TTACTACTGTGCAGGCGCTTATAGTAG | GGGGCCCAGGCACCCTGGTCACCGTCTCCTTA |
| | TAATAGTGATGTTAGGGCTTTCGGCG | (SEQ ID NO: 145) |
| | GAGGGACCGAGCTGGTGGTCAAA | |
| | (SEQ ID NO: 132) | |
| 17F22 | GCCCAAGTGCTGACCCAGACTGCATC | CAGGAGCAGCTGAAGGAGTCCGGAGGAGGCC |
| | GCCCGTGTCTGCAGCTGTGGGAAGCA | TGGTAACGCCTGGAGGAACCCTGACACTCAC |
| | CAGTCACCATCAATTGCCAGGCCAGT | CTGCACAGCCTCTGGATTCACCATCAATAGTT |
| | CAGAGTGTTTATAATAACAACAACTT | ACTTCATGAGCTGGGTCCGCCAGGCTCCAGGG |
| | AGCCTGGTATCAGCAGAAACCAGGGC | AAGGGACTGGAATGGATCGGAATCATTTATG |
| | AGCCTCCCAAGCTCCTGATCTATTATA | GTCGTGATAAGACATACTACGCGACCTGGAC |
| | CATCCACTCTGGCATCTGGGGTCTCAT | GAAAGGCCGATTCACCATCTCCAAAACCTCG |
| | CGCGGTTCAAAGGCAGTGGATCTGGG | ACCACGGTGGATCTGATAATCACCAGTCCGAC |
| | ACACAGTTCACTCTCACCATCAGCGG | AACCGAGGACACGGCCACCTATTTCTGTGCCA |
| | CATGCAGTGTGACGATGCTGCCACTT | GAAGTTCTAGTAGTGGTAGGGGTTTATATTAC |
| | ACTACTGTCAAGGCGAATTTGCTTGTA | GGTGGCATGGACCCCTGGGGCCCAGGGACCC |
| | GTACTGCTGATTGTCTTACTTTCGGCG | TCGTCACCGTCTCTTCA (SEQ ID NO: 146) |
| | GAGGGACCGAGGTGGTGGTCAAA | |
| | (SEQ ID NO: 133) | |
| 20K24 | GCGCTTGTGATGACCCAGACTCCAGC | CAGTCGTTGGAGGAGTCCGGGGGAGACCTGG |
| | CTCCGTGTCTGCAGCTGTGGGAGGCA | TCAAGCCTGGGGCATCCCTGACACTCACCTGC |
| | CAGTCACCATCAATTGCCAGGCCAGT | ACAGCCTCTGGATTCTCCTTCAGTAGCGGCTA |
| | GAGAGCATTAGCAGTTGGTTAGCCTG | CTACATGTGCTGGGTCCGCCAGGCTCCAGGGA |
| | GTATCAGCAGAAACCAGGGCAGCCTC | AGGGGCTGGAGTGGATCGCATGTATTTATGCT |
| | CCAAACTCCTGATCTATTATGCATCCA | GGTAGTGGTGCTAGAACTTACTACGCGAGCTG |
| | CTCTGGCATCTGGGGTCTCATCGCGGT | GGCGAAAGGCCGATTCACCATCTCCAAAACC |
| | TCAAAGGCAGTGGATCTAGGACAGAG | TCGTCGACCACGGTGACTCTGCAAATGACCAG |
| | TACACTCTTACCATCAGCGACCTGGA | TCTGACAGCCGCGGACACGGCCACCTATTTCT |
| | GTGTGCCGATGCTGCCACTTACTACTG | GTGCGAGAGACCCATATAGCGTGAATGATCC |
| | TGCAGGCTATGGAAGTGCTAATGATG | TGTTGGCGATACCTTGTGGGGCCCAGGCACCC |
| | ATAAAAATGGTTTCGGCGGAGGGACC | TGGTCACCGTCTCCTCA (SEQ ID NO: 147) |
| | GAGGTGGTGGTCAAA (SEQ ID NO: | |
| | 134) | |
| 20P19 | GCCTATGATATGACCCAGACTCCAGC | GAGCGCCTGGAGGAGTCCGGGGGAGACCTGG |
| | CTCTGTGGAGGTAGCTGTGGGAGGCA | TCAAGCCTGGGGCATCCCTGACACTCACCTGC |
| | CAGTCACCATCAAGTGCCAGGCCAGT | AAAGCCTCTGGAGCCGACTTCAATAACAACT |
| | CAGAGTATTAACAATGAATTGGCCTG | ACATAATGTGTTGGGTCCGCCAGGCTCCAGGG |
| | GTATCAGCAGAAACCAGGGCAGCCTC | AAGGGGCTGGAGTGGATCGCATGCATTAGCA |
| | CCAAGCTCCTGATCTATCTGGCATTCA | CTCTCACTACTGCCACTTACTACGCGACCTGG |
| | CTCTGGCATCTGGGGTCCCATCGCGGT | GCAAAAGGCCGATTCACCATCTCCACAACCTC |
| | TCAAAGGCAGTAGATCTGGGACAGAG | GTCGACCACGGTGACTCTGCAAATGACCAGC |
| | TTCACTCTCACCATTAGCGACCTGGAG | CTGACAGCCGCGGACACGGCCACCTATTTCTG |
| | TGTGCCGATGCTGCCACTTACTACTGT | TGCGGGAGATGGTTGGAGTGGTGATGGTGTG |
| | CAACAGGGTTATATTATTAGTGGTGTT | ATCACATTTAACTTGTGGGGCCCAGGCACCCT |
| | GATAATGTTTTCGGCGGAGGGACCGA | GGTCACCGTCTCCTCA (SEQ ID NO: 148) |
| | GGTGGTGGTCAAA (SEQ ID NO: 135) | |
| 19F24 | GCCCAAGTGCTGACCCAGACTCCATC | CAGTCGCTGGAGGAGTCCGGGGGTCGCCTGG |
| | CTCCGTGTCTGCAGCTGTGGGAGGCA | TCACGCCTGGGACACCCTTGACACTCACCTGC |
| | CAGTCACCATCAATTGCCAGTCCAGT | ACAGCCTCTGGATTCTCCCTCAGTAGCAGCTA |
| | CAGAGTGTTTGGGAGAAAAACTGGTT | CTGGATAAACTGGGTTCGCCAGGCTCCAGGG |
| | ATCCTGGTTTCAGCAGAAACCAGGGC | AAGGGCCTGGAATGGATCGCGATTATGCCGG |
| | AGCCTCCCAAGCTCCTGATCTACAGG | CAGGTGGGCGTCCTTACTACGCGACCTGGGCA |
| | GCATCCACTCTGGCATCTGGGGTCCC | AAAGGCCGATTCATCATCTCCAAAACCTCGAC |
| | ATCGCGGTTCAAGAGCAGTGGATCTG | CACGGTGGATCTGAAAATGACCAGTCCGACA |
| | GGACACAGTTCACTCTCACCATCAGC | ACCGAGGACACGGCCACCTATTTCTGTGCCAG |
| | GGCGTGCAGTGTGACGATGCTGCCGT | AGACGATATTGGTACTCCTGGTGGGGACATCT |
| | TTATTATTGTGCAGGCGCTTATAGTGT | GGGGCCCAGGCACCCTGGTCACCGTCTCCTTA |
| | TAATAGTGATGTTAGGGCTTTCGGCG | (SEQ ID NO: 149) |
| | GAGGGACCGAGCTGGTGGTCAAA | |
| | (SEQ ID NO: 136) | |

GalNAc moiety-specific mAbs

| | | |
|---|---|---|
| 14D4 | GCCATCGTGATGACCCAGACTCCATC | CAGTCGGTGGAGGAGTCCGGGGGTCGCCTGG |
| | TTCCAAGTCTGTCCCTGTGGGAGGCA | TCACGCCTGGGACACCCCTGACACTCACCTGC |
| | CAGTCACCATCAATTGCCAGTCCAGT | ACAGTCTCTAGAATCGACCTCAGTAGATATGT |
| | GAGAGTGTTTATGAGAACAACGACTT | GGTGGACTGGGTCCGCCAGGCTCCAGGGGAG |
| | ATCCTGGTATCAGCAGAAACCAGGGC | GGGCTGGAATGGATCGGAACCATTGGTTATG |
| | AGCCTCCCAAGCTCCTGATCTACTGG | GTAGCACATGGTACGCGAGCTGGGTGAAAGG |
| | GCATCCAGCCTGGCATCTGGGGTCCC | CCGATTCACCATCTCCAGAACCTCGACCACGG |
| | ATCGCGGTTCGAAGGCAGTGGATCTG | TGGATCTGAAAATGACCAGTCTGACAACCGA |
| | GGACACAGTTCACTCTCACCATCAGC | GGACACGGCCACCTATTTCTGTGCCAGGGGA |
| | AATGTGGTGTGTGACGATGCTGCCAC | AATGTTGGGAGTACTGGGGTCAGCATCTGGG |
| | TTACTACTGTGCAGGATATAAAAGTA | GCCCAGGCACGCTGGTCACCGTCTCCTTA |
| | TGAGTACTGATGGCTTTGCTTTCGGCG | (SEQ ID NO: 150) |
| | GAGGGACCGAGGTGGTGGTCAAA | |
| | (SEQ ID NO: 137) | |
| 16I3 | GCCATCGTGATGACCCAGACTCCATC | CAGTCGGTGGAGGAGTCCGGGGGTCGCCTGG |
| | TTCCAAGTCTGTCCCTGTGGGAGGCA | TCGCGCCTGGGACACCCCTGACACTCACCTGC |
| | CAGTCACCATCAGTTGCCAGGCCAGT | ACCGTCTCTGGATTCTCCCTCAGTTACTTTGG |
| | CAGAGTCTTTATAAGAACACCGACTT | AATGTACTGGGTCCGCCAGGCTCCAGGGAGG |
| | AGCCTGGTTTCAACAGAAACCAGGGC | GGGCTGGAATGGATCGGAACCATTGATAGTA |
| | AGCCTCCCAAACTCCTGATCTATTTTG | GTGATATCATATACTACGCGAGCTGGGCGAA |
| | CATCGAATCTGGCATCTGGAGTCACA | AGGCCGATTCACCATCTCCAAAACCTCGACCA |
| | TCGCGGTTCAAAGGCAGTGGATCTGG | CGGTGGATCTGAAAATCACCAGTCCGACAAC |
| | GACACAGTTCACTCTCACCATCAGCG | CGAGGACACGGCCACCTATTTCTGTGCCAGAT |
| | ATGTGGTGTGTGACGATGCTGCCACTT | CTGGTGGTGTGGCTGGTGGAGATAGCGTCTGG |
| | ACTACTGTGCAGGATATAAAAGTAGT | GGCCCAGGCACCCTGGTCACCGTCTCCTTA |
| | ACTACTGATGGGTTTGGTTTCGGCGG | (SEQ ID NO: 151) |
| | AGGGACCGAGGTGGTGGTCAAA (SEQ | |
| | ID NO: 138) | |
| 16A22 | GCGCAAGTGCTGGCCCAGACTCCATC | CAGTCACTGGAGGAGTCCGGGGGTCGCCTGG |
| | CTCCGTGTCTGCAGCTGTGGGAGGCA | TCACGCCTGGAGGATACCTGACACTCACCTGC |
| | CAGTCACCATCGATTGCCAGTCCAGT | ACAGTCTCTGGATTCTCCCTCAGCAGCTATGA |
| | CAGAGTGTTGGTAATAACGCCTATTT | CATGGGCTGGGTCCGCCAGGCTCCAGGGAAG |
| | ATCCTGGTATCAGCAGAAACCAGGGC | GGGCTGGAGTGGATCGGATACATTTTTATTAA |
| | AGCCTCCCAAGCTCCTGATCTATTATG | TGATAACACATACTACGCGACCTGGGCGAAA |
| | CATCCACTCTGGCATCTGGGGTCCCCT | GGCCGATTCACCATCTCCAAGACCTCGACCAC |
| | CCCGGTTCAGTGGCAGTGGATCTGGG | GATGGATCTGAAAATGACCAGTCTGACAACC |
| | ACACACTTCACTCTCACCATCAGCGG | GAGGACACGGCCACCTATTTCTGTGTCAGAGG |
| | CGTGCAGTGTGACGATGCTGCCACTT | ATATTTTGGCGGCATGGACCCCTGGGGCCCAG |
| | ACTACTGTCAAGCCTATTACCATGTTG | GGACCCTCGTCACCGTCTCTTCA (SEQ ID NO: |
| | GTGTTGCTGCTTTCGGCGGAGGGACC | 152) |
| | GAGGTGGTGGTCAAA (SEQ ID NO: | |
| | 139) | |
| 17D13 | GACGTCGTGATGACCCAGACTCCAGC | CAGTCGGTGGAGGAGTCCGGGGGTCGCCTGG |
| | CTCCGTGTCTGAATCTGTGGGAGGCA | TCACGCCTGGGGCATCCCTGACACTCACCTGC |
| | CAGTCACCATCAAGTGCCAGGCCAGT | ACAGTCTCTGGAATCGACTTCAGTACCAATGC |
| | CAGGATCTTTATAGTAATTGTTTATCC | AATGACCTGGGTCCGCCAGGCTCCAGGGAAG |
| | TGGTATCAGCAGAAACCAGGGCAGCG | GGGCTGGAATGGATCGGATATATTTATGAGG |
| | TCCCAAGCTCCTGATGTATCTGACATC | GTAGTGGTAATACATTCTACGCGAGCTGGGCG |
| | CACTCTGGCATCTGGGGTCCCATCGC | AAAGGCCGATTCACCATTTCCAGAACCTCGAC |
| | GGTTCAAAGGCAGTGGATCTGGGACA | CACGGTGGATTTGAAAATGACCAGTCTGACA |
| | GATTTCACTCTCACCATCAGCGACCTG | ATGGAGGACACGGCCACCTATTTCTGTGCCAG |
| | GAGTGTGCCGATGCTGCCACTTACTA | AGGATATCTCGGCGCCATGGACCCCTGGGGC |
| | CTGTCAAGGCTTCCATGGTTATGGTGT | CCAGGGACCCTCGTCACCGTCTCTTCA (SEQ |
| | TGGTGCCGCTTTCGGCGGAGGGACCG | ID NO: 153) |
| | AGGTGGTGGTCAAG (SEQ ID NO: 140) | |
| 18J5 | GACGTCGTGATGACCCAGACTCCAGC | CAGTCAGTGGAGGAGTCCGGGGGAGGCCTGG |
| | CTCCGTGGAGGCAGCTGTGGGAGGCA | TCAAGCCTGGGGCATCCCTGACACTCACCTGT |
| | CAGTCACCATCAAGTGCCAGGCCAGT | CAAGTCTCTGGATTCTCCCTCAGTGACCACTA |
| | CAGAGCATTAATAGTTGGTTATCCTG | CATGAGCTGGGTCCGCCAGGCTCCAGGGAAG |
| | GTATCAGCAGAAACCAGGGCAGCGTC | GGGCTGGAATGGGTCGCATATATTAGTGAGG |
| | CCAAACTCCTGATCTATGCTGCATCCA | GTGGTGCCACATACTACGCGAGCTGGGCAAA |
| | CTCTGGCATCTGGGGTCTCATCGCGGT | AGGCCGATTCACCATCTCCAAAACCTCGTCGA |
| | TCAAAGGCAGTAAATCTGGGACAGAG | CCACGGTGGATCTGAAAATGACCAGTCTGAC |
| | TTCACTCTCACCATCAGCGGTGTGCAG | AACCGAGGACACGGCCACCTATTTTTGTGCCA |
| | TGTGACGATGCTGCCACTTATTATTGT | GAGGATGGCTTGCTGCTTTTGATCCCTGGGGC |
| | CAAGGCTATGATGGTAGTAGTGGTAG | CCAGGCACCCTGGTCACCGTCTCCTCA (SEQ |
| | TGCTGCTAGTTTCGGCGGAGGGACCG | ID NO: 154) |
| | AGGTGGTGGTCAAA (SEQ ID NO: 141) | |

TABLE 4
Exemplary Light Chain and Heavy Chain Nucleic Acid Sequences for Anti-
Nucleic Acid and Anti-GalNAc Moiety Antibodies

| | | |
|---|---|---|
| Antibody | Light Chain Nucleic | |
| ID. | Acid Sequence | Heavy Chain Nucleic Acid Sequence |

Pan-specific mAbs

| | | |
|---|---|---|
| 14K10 | GCGCTTGTGATGACCCAGACTCC | CAGTCGCTGGAGGAGTCCGGGGGAGACCTGGTCA |
| | ATCCCCTGTGTCTGCAGCTGTGG | AGCCTGGGGCATCCCTGACACTCACCTGCACAGCC |
| | GAGGCACAGTCACCATCAACTGC | TCTGGATTCTCCTTCAGTAGCAGCTATTACATGTGG |
| | CAGGCCAGTCAGAGTGTTCTTAA | TGGGTCCGCCAGGCTCCAGGGAAGGGGCTGGAGT |
| | TAACAACTACTTATCCTGGTATC | GGATCGCATGCATTAATGGTGGTAGTCGTGGTACC |
| | AGCAGAAACCAGGGCAGCCTCCC | ACTTACTACGCGAGCTGGGCGAAAGGCCGATTCAC |
| | AAGCTCCTGATCTATTCTGCATCC | CATCTCCAAAACCTCGTCGACCACGGTGACTCTGC |
| | AAACTGGCAACTGGGGTCCCATC | AAATGACCAGTCTGACAGTCGCGGACACGGCCAC |
| | GCGGTTCAGCGGCAGTGGATCTG | CTATTTCTGTGCGAGAGATCCATATGGTTTTAGTG |
| | GGACACAGTTTACTCTCACCATC | GTAGTATTTATGCCTTGTGGGGCCCAGGCACCCTG |
| | AGCGGCGTGCAGTGTGACGATGC | GTCACCGTCTCCTCAGGGCAACCTAAGGCTCCATC |
| | TGCCACTTACTACTGTGCAGGCT | AGTCTTCCCACTGGCCCCCTGCTGCGGGGACACAC |
| | ATAAAAATTTCGGTAATGATGAT | CCAGCTCCACGGTGACCTTGGGCTGCCTGGTCAAA |
| | AATGCTTTCGGCGGAGGGACCGA | GGCTACCTCCCGGAGCCAGTGACCGTGACCTGGAA |
| | GGTGGTGGTCAAAGGTGATCCAG | CTCGGGCACCCTCACCAATGGGGTACGCACCTTCC |
| | TTGCACCTACTGTCCTCCTCTTCC | CGTCCGTCCGGCAGTCCTCAGGCCTCTACTCGCTG |
| | CACCATCTAGCGATGAGGTGGCA | AGCAGCGTGGTGAGCGTGACCTCAAGCAGCCAGC |
| | ACTGGAACAGTCACCATCGTGTG | CCGTCACCTGCAACGTGGCCCACCCAGCCACCAAC |
| | TGTGGCGAATAAATACTTTCCCG | ACCAAAGTGGACAAGACCGTTGCGCCCTCGACATG |
| | ATGTCACCGTCACCTGGGAGGTG | CAGCAAGCCCACGTGCCCACCCCCTGAACTCCTGG |
| | GATGGCACCACCCAAACAACTGG | GGGGACCGTCTGTCTTCATCTTCCCCCCAAAACCC |
| | CATCGAGAACAGTAAAACACCGC | AAGGACACCCTCATGATCTCACGCACCCCCGAGGT |
| | AGAATTCTGCAGATTGTACCTAC | CACATGCGTGGTGGTGGACGTGAGCCAGGATGAC |
| | AACCTCAGCAGCACTCTGACACT | CCCGAGGTGCAGTTCACATGGTACATAAACAACGA |
| | GACCAGCACACAGTACAACAGCC | GCAGGTGCGCACCGCCCGGCCGCCGCTACGGGAG |
| | ACAAAGAGTACACCTGCAAGGTG | CAGCAGTTCAACAGCACGATCCGCGTGGTCAGCAC |
| | ACCCAGGGCACGACCTCAGTCGT | CCTCCCCATCGCGCACCAGGACTGGCTGAGGGGCA |
| | CCAGAGCTTCAGTAGGAAGAACT | AGGAGTTCAAGTGCAAAGTCCACAACAAGGCACT |
| | GT (SEQ ID NO: 155) | CCCGGCCCCCATCGAGAAAACCATCTCCAAAGCCA |
| | | GAGGGCAGCCCCTGGAGCCGAAGGTCTACACCAT |
| | | GGGCCCTCCCCGGGAGGAGCTGAGCAGCAGGTCG |
| | | GTCAGCCTGACCTGCATGATCAACGGCTTCTACCC |
| | | TTCCGACATCTCGGTGGAGTGGGAGAAGAACGGG |
| | | AAGGCAGAGGACAACTACAAGACCACGCCGGCCG |
| | | TGCTGGACAGCGACGGCTCCTACTTCCTCTACAGC |
| | | AAGCTCTCAGTGCCCACGAGTGAGTGGCAGCGGG |
| | | GCGACGTCTTCACCTGCTCCGTGATGCACGAGGCC |
| | | TTGCACAACCACTACACGCAGAAGTCCATCT |
| | | CCCGCTCTCCGGGTAAA (SEQ ID NO: 168) |
| 14F4 | GCGCAAGTGCTGACCCAGACATC | CAGTCGCTGGAGGAGTCCGGGGGTCGCCTGGTCAC |
| | ATCCCCCGTGTCTGTAAATATGG | GCCTGGGACACCCCTGACACTCTCCTGCAAAGCCT |
| | GAGGCACAGTCACCATCAACTGC | CTGGACTCTCCCTCAGTGGCCACTACATGAGCTGG |
| | CAGTCCAGTCTGAGTGTTAATAG | GTCCGCCAGGCTCCAGGGAAGGGGCTGGAGTGGA |
| | GAGCGACTTATCCTGGTATCAGC | TCGGACACATTTATGGTAGTGGCCGTGGTTTATGG |
| | AGAAACCAGGGCAGCCTCCCAA | TACGCGAACTGGGCGAAAGGCCGATTCACCATCTC |
| | ACTCCTGATCTATCTGGCATCCA | CAAGACCTCGACCACGGTAGATCTGAAAATTATCA |
| | ATCTGGAATCTGGGGTCCCCTCG | GTCCGACAAACGAGGACACGGCCACCTATTTCTGT |
| | CGGTTCAAAGGCAGTGGATCTGG | GCCAGATATCGCCCTGTTGATTATGTGATGGACAT |
| | GACGCACTTCACTCTCACCATCA | CTGGGGCCCAGGCACCCTGGTCACCGTCTCGTTAG |
| | ACGGCGTGGAATGTGACGATGCT | GGCAACCTAAGGCTCCATCAGTCTTCCCACTGGCC |
| | GCCACTTACTACTGTGCAGGCGG | CCCTGCTGCGGGGACACACCCAGCTCCACGGTGAC |
| | TTATAGTAGTGGTAATGATAGGA | CTTGGGCTGCCTGGTCAAAGGCTACCTCCCGGAGC |
| | ATGCTTTCGGCGGAGGGACCGAG | CAGTGACCGTGACCTGGAACTCGGGCACCCTCACC |
| | GTGGTGGTCAAAGGTGATCCAGT | AATGGGGTACGCACCTTCCCGTCCGTCCGGCAGTC |
| | TGCACCTACTGTCCTCCTCTTCCC | CTCAGGCCTCTACTCGCTGAGCAGCGTGGTGAGCG |
| | ACCATCTAGCGATGAGGTGGCAA | TGACCTCAAGCAGCCAGCCCGTCACCTGCAACGTG |
| | CTGGAACAGTCACCATCGTGTGT | GCCCACCCAGCCACCAACACCAAAGTGGACAAGA |
| | GTGGCGAATAAATACTTTCCCGA | CCGTTGCGCCCTCGACATGCAGCAAGCCCACGTGC |
| | TGTCACCGTCACCTGGGAGGTGG | CCACCCCCTGAACTCCTGGGGGGACCGTCTGTCTT |
| | ATGGCACCACCCAAACAACTGGC | CATCTTCCCCCCAAAACCCAAGGACACCCTCATGA |
| | ATCGAGAACAGTAAAACACCGC | TCTCACGCACCCCCGAGGTCACATGCGTGGTGGTG |
| | AGAATTCTGCAGATTGTACCTAC | GACGTGAGCCAGGATGACCCCGAGGTGCAGTTCA |
| | AACCTCAGCAGCACTCTGACACT | CATGGTACATAAACAACGAGCAGGTGCGCACCGC |
| | GACCAGCACACAGTACAACAGCC | CCGGCCGCCGCTAC |
| | ACAAAGAGTACACCTGCAAGGTG | GGGAGCAGCAGTTCAACAGCACGATCCGCGTGGT |
| | ACCCAGGGCACGACCTCAGTCGT | CAGCACCCTCCCCATCGCGCACCAGGACTGGCTGA |
| | CCAGAGCTTCAGTAGGAAGAACT | GGGGCAAGGAGTTCAAGTGCAAAGTCCACAACAA |
| | GT (SEQ ID NO: 156) | GGCACTCCCGGCCCCCATCGAGAAAACCATCTCCA |
| | | AAGCCAGAGGGCAGCCCCTGGAGCCGAAGGTCTA |
| | | CACCATGGGCCCTCCCCGGGAGGAGCTGAGCAGC |
| | | AGGTCGGTCAGCCTGACCTGCATGATCAACGGCTT |
| | | CTACCCTTCCGACATCTCGGTGGAGTGGGAGAAGA |
| | | ACGGGAAGGCAGAGGACAACTACAAGACCACGCC |
| | | GGCCGTGCTGGACAGCGACGGCTCCTACTTCCTCT |
| | | ACAGCAAGCTCTCAGTGCCCACGAGTGAGTGGCA |
| | | GCGGGGCGACGTCTTCACCTGCTCCGTGATGCACG |
| | | AGGCCTTGCACAACCACTACACGCAGAAGTCCATC |
| | | TCCCGCTCTCCGGGTAAA |
| | | (SEQ ID NO: 169) |
| 5I17 | GCGCTTGTGATGACCCAGACTCC | CAGGAGCAGCTGGAGGAGTCCGGGGGAGACCTGG |
| | ATCCCCTGTGTCTGCAGCTGTGG | TCAAGCCTGAGGGATCCCTGACACTCACCTGCACA |
| | GAGGCACAGTCACCATCAGTTGC | GCCTCTGGATTCTCCTTCAGTAGCAACTACTACAT |
| | CAGGCCAGTCAGAGTGTTGCTAA | GTGCTGGGTCCGCCAGGCTCCAGGGAAGGGACTG |
| | TAACAACTACTTAGCCTGGTATC | GAGTGGATCGCATGCATTTATGCTGGTAGTAGTGG |
| | AGCAGAAACCAGGGCAGCCTCCC | TAGCACTTACTACGCGAGCTGGGCGAAAGGCCGGT |
| | AAGCTCCTGATTTACCAGGCATC | TCACCGTCTCCAAAACCTCGTCGACCACGGTGACT |
| | CACTCTGGCATCTGGGGTCCCAT | CTGCAAATGACCAGTCTGACAGCCGCGGACACGG |
| | CGCGGTTCAAAGGCAGTGGATCT | CCACCTATTTCTGTGCGAGAGAGCGTGAAAATTAT |
| | GGGACACAGTTCACTCTCACCAT | ATTGGTGTTGGTTACTATTTGTGGGGCCCAGGCAC |
| | CAGTGGCGTGGAGTGTGACGATG | CCTGGTCACCGTCTCCTCAGGGCAACCTAAGGCTC |
| | CTGCCACTTACTACTGTGCAGGC | CATCAGTCTTCCCACTGGCCCCCTGCTGCGGGGAC |
| | TATCGAAGTTATATTAATGCTGA | ACACCCAGCTCCACGGTGACCTTGGGCTGCCTGGT |
| | TAATGCTTTCGGCGGAGGGACCG | CAAAGGCTACCTCCCGGAGCCAGTGACCGTGACCT |
| | AGGTGGTGGTCGAAGGTGATCCA | GGAACTCGGGCACCCTCACCAATGGGGTACGCACC |
| | GTTGCACCTACTGTCCTCCTCTTC | TTCCCGTCCGTCCGGCAGTCCTCAGGCCTCTACTCG |
| | CCACCATCTAGCGATGAGGTGGC | CTGAGCAGCGTGGTGAGCGTGACCTCAAGCAGCC |
| | AACTGGAACAGTCACCATCGTGT | AGCCCGTCACCTGCAACGTGGCCCACCCAGCCACC |
| | GTGTGGCGAATAAATACTTTCCC | AACACCAAAGTGGACAAGACCGTTGCGCCCTCGA |
| | GATGTCACCGTCACCTGGGAGGT | CATGCAGCAAGCCCACGTGCCCACCCCCTGAACTC |
| | GGATGGCACCACCCAAACAACTG | CTGGGGGGACCGTCTGTCTTCATCTTCCCCCCAAA |
| | GCATCGAGAACAGTAAAACACC | ACCCAAGGACACCCTCATGATCTCACGCACCCCCG |
| | GCAGAATTCTGCAGATTGTACCT | AGGTCACATGCGTGGTGGTGGACGTGAGCCAGGA |
| | ACAACCTCAGCAGCACTCTGACA | TGACCCCGAGGTGCAGTTCACATGGTACATAAACA |
| | CTGACCAGCACACAGTACAACAG | ACGAGCAGGTGCGCACCGCCCGGCCGCCGCTACG |
| | CCACAAAGAGTACACCTGCAAGG | GGAGCAGCAGTTCAACAGCACGATCCGCGTGGTC |
| | TGACCCAGGGCACGACCTCAGTC | AGCACCCTCCCCATCGCGCACCAGGACTGGCTGAG |
| | GTCCAGAGCTTCAGTAGGAAGAA | GGGCAAGGAGTTCAAGTGCAAAGTCCACAACAAG |
| | CTGT (SEQ ID NO: 157) | GCACTCCCGGCCCCCATCGAGAAAACCATCTCCAA |
| | | AGCCAGAGGGCAGCCCCTGGAGCCGAAGGTCTAC |
| | | ACCATGGGCCCTCCCCGGGAGGAGCTGAGCAGCA |
| | | GGTCGGTCAGCCTGACCTGCATGATCAACGGCTTC |
| | | TACCCTTCCGACATCTCGGTGGAGTGGGAGAAGAA |
| | | CGGGAAGGCAGAGGACAACTACAAGACCACGCCG |
| | | GCCGTGCTGGACAGCGACGGCTCCTACTTCCTCTA |
| | | CAGCAAGCTCTCAGTGCCCACGAGTGAGTGGCAGC |
| | | GGGGCGACGTCTTCACCTGCTCCGTGATGCACGAG |
| | | GCCTTGCACAACCACTACACGCAGAAGTCCATCT |
| | | CCCGCTCTCCGGGTAAA (SEQ ID NO: 170) |

1851 RNAi construct-specific mAbs

| | | |
|---|---|---|
| 17K13 | GCCCAAGTGCTGACCCAGACTCC | GAGCAGCTGAAGGAGTCCGGGGGTCGCCTGGTCA |
| | ATCCTCCGTGTCTGCAGCTGTGG | CGCCTGGGACACCCCTGACACTCACCTGCACCGTC |
| | GAGGCACAGTCACCATCAATTGC | TCTGGCTTCTCCCTCAGTGGCAGCTATTGGATAAA |
| | CAGTCCAGTCAGAGTGTTTGGCA | TTGGGTTCGCCAGGCTCCAGGGAAGGGGCTGGAAT |
| | GAAAAACTGGTTATCCTGGTTTC | GGATCGCGATTATTGCGGCAGGTGGGCGTATATGG |
| | AGCAGAAACCAGGGCAGCCTCCC | TACGCGAGCTCGGTGAAAGGCCGATTCACCATCTC |
| | AAGCTCCTGATCTACAGGGCGTC | CAAAACCTCGACCACGGTGGATCTGAAAATGACC |
| | CACTCTGGCATCTGGGGTCCCAT | AGTCCGACAACCGAGGACACGGCCACCTATTTCTG |
| | CGCGGTTCAAGAGCAGTGGATCT | TGCCAGAGACGATATTGGTATTCCTGGTGGGGACA |
| | GGGACACAGTTCACTCTCACCAT | TCTGGGGCCCAGGCACCCTGGTCACCGTCTCCTTA |
| | CAGCGGCGTGCAGTGTGACGATG | GGGCAACCTAAGGCTCCATCAGTCTTCCCACTGGC |
| | CTGCCACTTACTACTGTGCAGGC | CCCCTGCTGCGGGGACACACCCAGCTCCACGGTGA |
| | GCTTATAGTAGTAATAGTGATGT | CCTTGGGCTGCCTGGTCAAAGGCTACCTCCCGGAG |
| | TAGGGCTTTCGGCGGAGGGACCG | CCAGTGACCGTGACCTGGAACTCGGGCACCCTCAC |
| | AGCTGGTGGTCAAAGGTGATCCA | CAATGGGGTACGCACCTTCCCGTCCGTCCGGCAGT |
| | GTTGCACCTACTGTCCTCCTCTTC | CCTCAGGCCTCTACTCGCTGAGCAGCGTGGTGAGC |
| | CCACCATCTAGCGATGAGGTGGC | GTGACCTCAAGCAGCCAGCCCGTCACCTGCAACGT |
| | AACTGGAACAGTCACCATCGTGT | GGCCCACCCAGCCACCAACACCAAAGTGGACAAG |
| | GTGTGGCGAATAAATACTTTCCC | ACCGTTGCGCCCTCGACATGCAGCAAGCCCACGTG |
| | GATGTCACCGTCACCTGGGAGGT | CCCACCCCCTGAACTCCTGGGGGGACCGTCTGTCT |
| | GGATGGCACCACCCAAACAACTG | TCATCTTCCCCCCAAAACCCAAGGACACCCTCATG |
| | GCATCGAGAACAGTAAAACACC | ATCTCACGCACCCCCGAGGTCACATGCGTGGTGGT |
| | GCAGAATTCTGCAGATTGTACCT | GGACGTGAGCCAGGATGACCCCGAGGTGCAGTTC |
| | ACAACCTCAGCAGCACTCTGACA | ACATGGTACATAAACAACGAGCAGGTGCGCACCG |
| | CTGACCAGCACACAGTACAACAG | CCCGGCCGCCGCTACGGGAGCAGCAGTTCAACAG |
| | CCACAAAGAGTACACCTGCAAGG | CACGATCCGCGTGGTCAGCACCCTCCCCATCGCGC |
| | TGACCCAGGGCACGACCTCAGTC | ACCAGGACTGGCTGAGGGGCAAGGAGTTCAAGTG |
| | GTCCAGAGCTTCAGTAGGAAGAA | CAAAGTCCACAACAAGGCACTCCCGGCCCCCATCG |
| | CTGT (SEQ ID NO: 158) | AGAAAACCATCTCCAAAGCCAGAGGGCAGCCCCT |
| | | GGAGCCGAAGGTCTACACCATGGGCCCTCCCCGGG |
| | | AGGAGCTGAGCAGCAGGTCGGTCAGCCTGACCTG |
| | | CATGATCAACGGCTTCTACCCTTCCGACATCTCGG |
| | | TGGAGTGGGAGAAGAACGGGAAGGCAGAGGACA |
| | | ACTACAAGACCACGCCGGCCGTGCTGGACAGCGA |
| | | CGGCTCCTACTTCCTCTACAGCAAGCTCTCAGTGC |
| | | CCACGAGTGAGTGGCAGCGGGGCGACGTCTTCACC |
| | | TGCTCCGTGATGCACGAGGCCTTGCACAACCACTA |
| | | CACGCAGAAGTCCATCT CCCGCTCTCCGGGTAAA |
| | | (SEQ ID NO: 171) |
| 17F22 | GCCCAAGTGCTGACCCAGACTGC | CAGGAGCAGCTGAAGGAGTCCGGAGGAGGCCTGG |
| | ATCGCCCGTGTCTGCAGCTGTGG | TAACGCCTGGAGGAACCCTGACACTCACCTGCACA |
| | GAAGCACAGTCACCATCAATTGC | GCCTCTGGATTCACCATCAATAGTTACTTCATGAG |
| | CAGGCCAGTCAGAGTGTTTATAA | CTGGGTCCGCCAGGCTCCAGGGAAGGGACTGGAA |
| | TAACAACAACTTAGCCTGGTATC | TGGATCGGAATCATTTATGGTCGTGATAAGACATA |
| | AGCAGAAACCAGGGCAGCCTCCC | CTACGCGACCTGGACGAAAGGCCGATTCACCATCT |
| | AAGCTCCTGATCTATTATACATC | CCAAAACCTCGACCACGGTGGATCTGATAATCACC |
| | CACTCTGGCATCTGGGGTCTCAT | AGTCCGACAACCGAGGACACGGCCACCTATTTCTG |
| | CGCGGTTCAAAGGCAGTGGATCT | TGCCAGAAGTTCTAGTAGTGGTAGGGGTTTATATT |
| | GGGACACAGTTCACTCTCACCAT | ACGGTGGCATGGACCCCTGGGGCCCAGGGACCCTC |
| | CAGCGGCATGCAGTGTGACGATG | GTCACCGTCTCTTCAGGGCAACCTAAGGCTCCATC |
| | CTGCCACTTACTACTGTCAAGGC | AGTCTTCCCACTGGCCCCCTGCTGCGGGGACACAC |
| | GAATTTGCTTGTAGTACTGCTGA | CCAGCTCCACGGTGACCTTGGGCTGCCTGGTCAAA |
| | TTGTCTTACTTTCGGCGGAGGGA | GGCTACCTCCCGGAGCCAGTGACCGTGACCTGGAA |
| | CCGAGGTGGTGGTCAAAGGTGAT | CTCGGGCACCCTCACCAATGGGGTACGCACCTTCC |
| | CCAGTTGCACCTACTGTCCTCCTC | CGTCCGTCCGGCAGTCCTCAGGCCTCTACTCGCTG |
| | TTCCCACCATCTAGCGATGAGGT | AGCAGCGTGGTGAGCGTGACCTCAAGCAGCCAGC |
| | GGCAACTGGAACAGTCACCATCG | CCGTCACCTGCAACGTGGCCCACCCAGCCACCAAC |
| | TGTGTGTGGCGAATAAATACTTT | ACCAAAGTGGACAAGACCGTTGCGCCCTCGACATG |
| | CCCGATGTCACCGTCACCTGGGA | CAGCAAGCCCACGTGCCCACCCCCTGAACTCCTGG |
| | GGTGGATGGCACCACCCAAACAA | GGGGACCGTCTGTCTTCATCTTCCCCCCAAAACCC |
| | CTGGCATCGAGAACAGTAAAACA | AAGGACACCCTCATGATCTCACGCACCCCCGAGGT |
| | CCGCAGAATTCTGCAGATTGTAC | CACATGCGTGGTGGTGGACGTGAGCCAGGATGAC |
| | CTACAACCTCAGCAGCACTCTGA | CCCGAGGTGCAGTTCACATGGTACATAAACAACGA |
| | CACTGACCAGCACACAGTACAAC | GCAGGTGCGCACCGCCCGGCCGCCGCTACGGGAG |
| | AGCCACAAAGAGTACACCTGCAA | CAGCAGTTCAACAGCACGATCCGCGTGGTCAGCAC |
| | GGTGACCCAGGGCACGACCTCAG | CCTCCCCATCGCGCACCAGGACTGGCTGAGGGGCA |
| | TCGTCCAGAGCTTCAGTAGGAAG | AGGAGTTCAAGTGCAAAGTCCACAACAAGGCACT |
| | AACTGT (SEQ ID NO: 159) | CCCGGCCCCCATCGAGAAAACCATCTCCAAAGCCA |
| | | GAGGGCAGCCCCTGGAGCCGAAGGTCTACACCAT |
| | | GGGCCCTCCCCGGGAGGAGCTGAGCAGCAGGTCG |
| | | GTCAGCCTGACCTGCATGATCAACGGCTTCTACCC |
| | | TTCCGACATCTCGGTGGAGTGGGAGAAGAACGGG |
| | | AAGGCAGAGGACAACTACAAGACCACGCCGGCCG |
| | | TGCTGGACAGCGACGGCTCCTACTTCCTCTACAGC |
| | | AAGCTCTCAGTGCCCACGAGTGAGTGGCAGCGGG |
| | | GCGACGTCTTCACCTGCTCCGTGATGCACGAGGCC |
| | | TTGCACAACCACTACACGCAGAAGTCCATCT |
| | | CCCGCTCTCCGGGTAAA (SEQ ID NO: 172) |
| 20K24 | GCGCTTGTGATGACCCAGACTCC | CAGTCGTTGGAGGAGTCCGGGGGAGACCTGGTCA |
| | AGCCTCCGTGTCTGCAGCTGTGG | AGCCTGGGGCATCCCTGACACTCACCTGCACAGCC |
| | GAGGCACAGTCACCATCAATTGC | TCTGGATTCTCCTTCAGTAGCGGCTACTACATGTGC |
| | CAGGCCAGTGAGAGCATTAGCAG | TGGGTCCGCCAGGCTCCAGGGAAGGGGCTGGAGT |
| | TTGGTTAGCCTGGTATCAGCAGA | GGATCGCATGTATTTATGCTGGTAGTGGTGCTAGA |
| | AACCAGGGCAGCCTCCCAAACTC | ACTTACTACGCGAGCTGGGCGAAAGGCCGATTCAC |
| | CTGATCTATTATGCATCCACTCTG | CATCTCCAAAACCTCGTCGACCACGGTGACTCTGC |
| | GCATCTGGGGTCTCATCGCGGTT | AAATGACCAGTCTGACAGCCGCGGACACGGCCAC |
| | CAAAGGCAGTGGATCTAGGACA | CTATTTCTGTGCGAGAGACCCATATAGCGTGAATG |
| | GAGTACACTCTTACCATCAGCGA | ATCCTGTTGGCGATACCTTGTGGGGCCCAGGCACC |
| | CCTGGAGTGTGCCGATGCTGCCA | CTGGTCACCGTCTCCTCAGGGCAACCTAAGGCTCC |
| | CTTACTACTGTGCAGGCTATGGA | ATCAGTCTTCCCACTGGCCCCCTGCTGCGGGGACA |
| | AGTGCTAATGATGATAAAAATGG | CACCCAGCTCCACGGTGACCTTGGGCTGCCTGGTC |
| | TTTCGGCGGAGGGACCGAGGTGG | AAAGGCTACCTCCCGGAGCCAGTGACCGTGACCTG |
| | TGGTCAAAGGTGATCCAGTTGCA | GAACTCGGGCACCCTCACCAATGGGGTACGCACCT |
| | CCTACTGTCCTCCTCTTCCCACCA | TCCCGTCCGTCCGGCAGTCCTCAGGCCTCTACTCG |
| | TCTAGCGATGAGGTGGCAACTGG | CTGAGCAGCGTGGTGAGCGTGACCTCAAGCAGCC |
| | AACAGTCACCATCGTGTGTGTGG | AGCCCGTCACCTGCAACGTGGCCCACCCAGCCACC |
| | CGAATAAATACTTTCCCGATGTC | AACACCAAAGTGGACAAGACCGTTGCGCCCTCGA |
| | ACCGTCACCTGGGAGGTGGATGG | CATGCAGCAAGCCCACGTGCCCACCCCCTGAACTC |
| | CACCACCCAAACAACTGGCATCG | CTGGGGGGACCGTCTGTCTTCATCTTCCCCCCAAA |
| | AGAACAGTAAAACACCGCAGAA | ACCCAAGGACACCCTCATGATCTCACGCACCCCCG |
| | TTCTGCAGATTGTACCTACAACC | AGGTCACATGCGTGGTGGTGGACGTGAGCCAGGA |
| | TCAGCAGCACTCTGACACTGACC | TGACCCCGAGGTGCAGTTCACATGGTACATAAACA |
| | AGCACACAGTACAACAGCCACA | ACGAGCAGGTGCGCACCGCCCGGCCGCCGCTACG |
| | AAGAGTACACCTGCAAGGTGACC | GGAGCAGCAGTTCAACAGCACGATCCGCGTGGTC |
| | CAGGGCACGACCTCAGTCGTCCA | AGCACCCTCCCCATCGCGCACCAGGACTGGCTGAG |
| | GAGCTTCAGTAGGAAGAACTGT | GGGCAAGGAGTTCAAGTGCAAAGTCCACAACAAG |
| | (SEQ ID NO: 160) | GCACTCCCGGCCCCCATCGAGAAAACCATCTCCAA |
| | | AGCCAGAGGGCAGCCCCTGGAGCCGAAGGTCTAC |
| | | ACCATGGGCCCTCCCCGGGAGGAGCTGAGCAGCA |
| | | GGTCGGTCAGCCTGACCTGCATGATCAACGGCTTC |
| | | TACCCTTCCGACATCTCGGTGGAGTGGGAGAAGAA |
| | | CGGGAAGGCAGAGGACAACTACAAGACCACGCCG |
| | | GCCGTGCTGGACAGCGACGGCTCCTACTTCCTCTA |
| | | CAGCAAGCTCTCAGTGCCCACGAGTGAGTGGCAGC |
| | | GGGGCGACGTCTTCACCTGCTCCGTGATGCACGAG |
| | | GCCTTGCACAACCACTACACGCAGAAGTCCATCT |
| | | CCCGCTCTCCGGGTAAA (SEQ ID NO: 173) |
| 20P19 | GCCTATGATATGACCCAGACTCC | GAGCGCCTGGAGGAGTCCGGGGGAGACCTGGTCA |
| | AGCCTCTGTGGAGGTAGCTGTGG | AGCCTGGGGCATCCCTGACACTCACCTGCAAAGCC |
| | GAGGCACAGTCACCATCAAGTGC | TCTGGAGCCGACTTCAATAACAACTACATAATGTG |
| | CAGGCCAGTCAGAGTATTAACAA | TTGGGTCCGCCAGGCTCCAGGGAAGGGGCTGGAG |
| | TGAATTGGCCTGGTATCAGCAGA | TGGATCGCATGCATTAGCACTCTCACTACTGCCAC |
| | AACCAGGGCAGCCTCCCAAGCTC | TTACTACGCGACCTGGGCAAAAGGCCGATTCACCA |
| | CTGATCTATCTGGCATTCACTCTG | TCTCCACAACCTCGTCGACCACGGTGACTCTGCAA |
| | GCATCTGGGGTCCCATCGCGGTT | ATGACCAGCCTGACAGCCGCGGACACGGCCACCT |
| | CAAAGGCAGTAGATCTGGGACA | ATTTCTGTGCGGGAGATGGTTGGAGTGGTGATGGT |
| | GAGTTCACTCTCACCATTAGCGA | GTGATCACATTTAACTTGTGGGGCCCAGGCACCCT |
| | CCTGGAGTGTGCCGATGCTGCCA | GGTCACCGTCTCCTCAGGGCAACCTAAGGCTCCAT |
| | CTTACTACTGTCAACAGGGTTAT | CAGTCTTCCCACTGGCCCCCTGCTGCGGGGACACA |
| | ATTATTAGTGGTGTTGATAATGTT | CCCAGCTCCACGGTGACCTTGGGCTGCCTGGTCAA |
| | TTCGGCGGAGGGACCGAGGTGGT | AGGCTACCTCCCGGAGCCAGTGACCGTGACCTGGA |
| | GGTCAAAGGTGATCCAGTTGCAC | ACTCGGGCACCCTCACCAATGGGGTACGCACCTTC |
| | CTACTGTCCTCCTCTTCCCACCAT | CCGTCCGTCCGGCAGTCCTCAGGCCTCTACTCGCT |
| | CTAGCGATGAGGTGGCAACTGGA | GAGCAGCGTGGTGAGCGTGACCTCAAGCAGCCAG |
| | ACAGTCACCATCGTGTGTGTGGC | CCCGTCACCTGCAACGTGGCCCACCCAGCCACCAA |
| | GAATAAATACTTTCCCGATGTCA | CACCAAAGTGGACAAGACCGTTGCGCCCTCGACAT |
| | CCGTCACCTGGGAGGTGGATGGC | GCAGCAAGCCCACGTGCCCACCCCCTGAACTCCTG |
| | ACCACCCAAACAACTGGCATCGA | GGGGGACCGTCTGTCTTCATCTTCCCCCCAAAACC |
| | GAACAGTAAAACACCGCAGAATT | CAAGGACACCCTCATGATCTCACGCACCCCCGAGG |
| | CTGCAGATTGTACCTACAACCTC | TCACATGCGTGGTGGTGGACGTGAGCCAGGATGAC |
| | AGCAGCACTCTGACACTGACCAG | CCCGAGGTGCAGTTCACATGGTACATAAACAACGA |
| | CACACAGTACAACAGCCACAAA | GCAGGTGCGCACCGCCCGGCCGCCGCTACGGGAG |
| | GAGTACACCTGCAAGGTGACCCA | CAGCAGTTCAACAGCACGATCCGCGTGGTCAGCAC |
| | GGGCACGACCTCAGTCGTCCAGA | CCTCCCCATCGCGCACCAGGACTGGCTGAGGGGCA |
| | GCTTCAGTAGGAAGAACTGT | AGGAGTTCAAGTGCAAAGTCCACAACAAGGCACT |
| | (SEQ ID NO: 161) | CCCGGCCCCCATCGAGAAAACCATCTCCAAAGCCA |
| | | GAGGGCAGCCCCTGGAGCCGAAGGTCTACACCAT |
| | | GGGCCCTCCCCGGGAGGAGCTGAGCAGCAGGTCG |
| | | GTCAGCCTGACCTGCATGATCAACGGCTTCTACCC |
| | | TTCCGACATCTCGGTGGAGTGGGAGAAGAACGGG |
| | | AAGGCAGAGGACAACTACAAGACCACGCCGGCCG |
| | | TGCTGGACAGCGACGGCTCCTACTTCCTCTACAGC |
| | | AAGCTCTCAGTGCCCACGAGTGAGTGGCAGCGGG |
| | | GCGACGTCTTCACCTGCTCCGTGATGCACGAGGCC |
| | | TTGCACAACCACTACACGCAGAAGTCCATCT |
| | | CCCGCTCTCCGGGTAAA (SEQ ID NO: 174) |
| 19F24 | GCCCAAGTGCTGACCCAGACTCC | CAGTCGCTGGAGGAGTCCGGGGGTCGCCTGGTCAC |
| | ATCCTCCGTGTCTGCAGCTGTGG | GCCTGGGACACCCTTGACACTCACCTGCACAGCCT |
| | GAGGCACAGTCACCATCAATTGC | CTGGATTCTCCCTCAGTAGCAGCTACTGGATAAAC |
| | CAGTCCAGTCAGAGTGTTTGGGA | TGGGTTCGCCAGGCTCCAGGGAAGGGCCTGGAAT |
| | GAAAAACTGGTTATCCTGGTTTC | GGATCGCGATTATGCCGGCAGGTGGGCGTCCTTAC |
| | AGCAGAAACCAGGGCAGCCTCCC | TACGCGACCTGGGCAAAAGGCCGATTCATCATCTC |
| | AAGCTCCTGATCTACAGGGCATC | CAAAACCTCGACCACGGTGGATCTGAAAATGACC |
| | CACTCTGGCATCTGGGGTCCCAT | AGTCCGACAACCGAGGACACGGCCACCTATTTCTG |
| | CGCGGTTCAAGAGCAGTGGATCT | TGCCAGAGACGATATTGGTACTCCTGGTGGGGACA |
| | GGGACACAGTTCACTCTCACCAT | TCTGGGGCCCAGGCACCCTGGTCACCGTCTCCTTA |
| | CAGCGGCGTGCAGTGTGACGATG | GGGCAACCTAAGGCTCCATCAGTCTTCCCACTGGC |
| | CTGCCGTTTATTATTGTGCAGGC | CCCCTGCTGCGGGGACACACCCAGCTCCACGGTGA |
| | GCTTATAGTGTTAATAGTGATGT | CCTTGGGCTGCCTGGTCAAAGGCTACCTCCCGGAG |
| | TAGGGCTTTCGGCGGAGGGACCG | CCAGTGACCGTGACCTGGAACTCGGGCACCCTCAC |
| | AGCTGGTGGTCAAAGGTGATCCA | CAATGGGGTACGCACCTTCCCGTCCGTCCGGCAGT |
| | GTTGCACCTACTGTCCTCCTCTTC | CCTCAGGCCTCTACTCGCTGAGCAGCGTGGTGAGC |
| | CCACCATCTAGCGATGAGGTGGC | GTGACCTCAAGCAGCCAGCCCGTCACCTGCAACGT |
| | AACTGGAACAGTCACCATCGTGT | GGCCCACCCAGCCACCAACACCAAAGTGGACAAG |
| | GTGTGGCGAATAAATACTTTCCC | ACCGTTGCGCCCTCGACATGCAGCAAGCCCACGTG |
| | GATGTCACCGTCACCTGGGAGGT | CCCACCCCCTGAACTCCTGGGGGGACCGTCTGTCT |
| | GGATGGCACCACCCAAACAACTG | TCATCTTCCCCCCAAAACCCAAGGACACCCTCATG |
| | GCATCGAGAACAGTAAAACACC | ATCTCACGCACCCCCGAGGTCACATGCGTGGTGGT |
| | GCAGAATTCTGCAGATTGTACCT | GGACGTGAGCCAGGATGACCCCGAGGTGCAGTTC |
| | ACAACCTCAGCAGCACTCTGACA | ACATGGTACATAAACAACGAGCAGGTGCGCACCG |
| | CTGACCAGCACACAGTACAACAG | CCCGGCCGCCGCTACGGGAGCAGCAGTTCAACAG |
| | CCACAAAGAGTACACCTGCAAGG | CACGATCCGCGTGGTCAGCACCCTCCCCATCGCGC |
| | TGACCCAGGGCACGACCTCAGTC | ACCAGGACTGGCTGAGGGGCAAGGAGTTCAAGTG |
| | GTCCAGAGCTTCAGTAGGAAGAA | CAAAGTCCACAACAAGGCACTCCCGGCCCCCATCG |
| | CTGT (SEQ ID NO: 162) | AGAAAACCATCTCCAAAGCCAGAGGGCAGCCCCT |
| | | GGAGCCGAAGGTCTACACCATGGGCCCTCCCCGGG |
| | | AGGAGCTGAGCAGCAGGTCGGTCAGCCTGACCTG |
| | | CATGATCAACGGCTTCTACCCTTCCGACATCTCGG |
| | | TGGAGTGGGAGAAGAACGGGAAGGCAGAGGACA |
| | | ACTACAAGACCACGCCGGCCGTGCTGGACAGCGA |
| | | CGGCTCCTACTTCCTCTACAGCAAGCTCTCAGTGC |
| | | CCACGAGTGAGTGGCAGCGGGGCGACGTCTTCACC |
| | | TGCTCCGTGATGCACGAGGCCTTGCACAACCACTA |
| | | CACGCAGAAGTCCATCTCCCGCTCTCCGGGTAAA |
| | | (SEQ ID NO: 175) |

GalNAc moiety-specific mAbs

| | | |
|---|---|---|
| 14D4 | GCCATCGTGATGACCCAGACTCC | CAGTCGGTGGAGGAGTCCGGGGGTCGCCTGGTCAC |
| | ATCTTCCAAGTCTGTCCCTGTGG | GCCTGGGACACCCCTGACACTCACCTGCACAGTCT |
| | GAGGCACAGTCACCATCAATTGC | CTAGAATCGACCTCAGTAGATATGTGGTGGACTGG |
| | CAGTCCAGTGAGAGTGTTTATGA | GTCCGCCAGGCTCCAGGGGAGGGGCTGGAATGGA |
| | GAACAACGACTTATCCTGGTATC | TCGGAACCATTGGTTATGGTAGCACATGGTACGCG |
| | AGCAGAAACCAGGGCAGCCTCCC | AGCTGGGTGAAAGGCCGATTCACCATCTCCAGAAC |
| | AAGCTCCTGATCTACTGGGCATC | CTCGACCACGGTGGATCTGAAAATGACCAGTCTGA |
| | CAGCCTGGCATCTGGGGTCCCAT | CAACCGAGGACACGGCCACCTATTTCTGTGCCAGG |
| | CGCGGTTCGAAGGCAGTGGATCT | GGAAATGTTGGGAGTACTGGGGTCAGCATCTGGG |
| | GGGACACAGTTCACTCTCACCAT | GCCCAGGCACGCTGGTCACCGTCTCCTTAGGGCAA |
| | CAGCAATGTGGTGTGTGACGATG | CCTAAGGCTCCATCAGTCTTCCCACTGGCCCCCTG |
| | CTGCCACTTACTACTGTGCAGGA | CTGCGGGGACACACCCAGCTCCACGGTGACCTTGG |
| | TATAAAAGTATGAGTACTGATGG | GCTGCCTGGTCAAAGGCTACCTCCCGGAGCCAGTG |
| | CTTTGCTTTCGGCGGAGGGACCG | ACCGTGACCTGGAACTCGGGCACCCTCACCAATGG |
| | AGGTGGTGGTCAAAGGTGATCCA | GGTACGCACCTTCCCGTCCGTCCGGCAGTCCTCAG |
| | GTTGCACCTACTGTCCTCCTCTTC | GCCTCTACTCGCTGAGCAGCGTGGTGAGCGTGACC |
| | CCACCATCTAGCGATGAGGTGGC | TCAAGCAGCCAGCCCGTCACCTGCAACGTGGCCCA |
| | AACTGGAACAGTCACCATCGTGT | CCCAGCCACCAACACCAAAGTGGACAAGACCGTT |
| | GTGTGGCGAATAAATACTTTCCC | GCGCCCTCGACATGCAGCAAGCCCACGTGCCCACC |
| | GATGTCACCGTCACCTGGGAGGT | CCCTGAACTCCTGGGGGGACCGTCTGTCTTCATCTT |
| | GGATGGCACCACCCAAACAACTG | CCCCCCAAAACCCAAGGACACCCTCATGATCTCAC |
| | GCATCGAGAACAGTAAAACACC | GCACCCCCGAGGTCACATGCGTGGTGGTGGACGTG |
| | GCAGAATTCTGCAGATTGTACCT | AGCCAGGATGACCCCGAGGTGCAGTTCACATGGTA |
| | ACAACCTCAGCAGCACTCTGACA | CATAAACAACGAGCAGGTGCGCACCGCCCGGCCG |
| | CTGACCAGCACACAGTACAACAG | CCGCTACGGGAGCAGCAGTTCAACAGCACGATCC |
| | CCACAAAGAGTACACCTGCAAGG | GCGTGGTCAGCACCCTCCCCATCGCGCACCAGGAC |
| | TGACCCAGGGCACGACCTCAGTC | TGGCTGAGGGGCAAGGAGTTCAAGTGCAAAGTCC |
| | GTCCAGAGCTTCAGTAGGAAGAA | ACAACAAGGCACTCCCGGCCCCCATCGAGAAAAC |
| | CTGT (SEQ ID NO: 163) | CATCTCCAAAGCCAGAGGGCAGCCCCTGGAGCCG |
| | | AAGGTCTACACCATGGGCCCTCCCCGGGAGGAGCT |
| | | GAGCAGCAGGTCGGTCAGCCTGACCTGCATGATCA |
| | | ACGGCTTCTACCCTTCCGACATCTCGGTGGAGTGG |
| | | GAGAAGAACGGGAAGGCAGAGGACAACTACAAG |
| | | ACCACGCCGGCCGTGCTGGACAGCGACGGCTCCTA |
| | | CTTCCTCTACAGCAAGCTCTCAGTGCCCACGAGTG |
| | | AGTGGCAGCGGGGCGACGTCTTCACCTGCTCCGTG |
| | | ATGCACGAGGCCTTGCACAACCACTACACGCAGA |
| | | AGTCCATCTCCCGCTCTCCGGGTAAA |
| | | (SEQ ID NO: 176) |
| 16I3 | GCCATCGTGATGACCCAGACTCC | CAGTCGGTGGAGGAGTCCGGGGGTCGCCTGGTCGC |
| | ATCTTCCAAGTCTGTCCCTGTGG | GCCTGGGACACCCCTGACACTCACCTGCACCGTCT |
| | GAGGCACAGTCACCATCAGTTGC | CTGGATTCTCCCTCAGTTACTTTGGAATGTACTGGG |
| | CAGGCCAGTCAGAGTCTTTATAA | TCCGCCAGGCTCCAGGGAGGGGGCTGGAATGGAT |
| | GAACACCGACTTAGCCTGGTTTC | CGGAACCATTGATAGTAGTGATATCATATACTACG |
| | AACAGAAACCAGGGCAGCCTCCC | CGAGCTGGGCGAAAGGCCGATTCACCATCTCCAAA |
| | AAACTCCTGATCTATTTTGCATCG | ACCTCGACCACGGTGGATCTGAAAATCACCAGTCC |
| | AATCTGGCATCTGGAGTCACATC | GACAACCGAGGACACGGCCACCTATTTCTGTGCCA |
| | GCGGTTCAAAGGCAGTGGATCTG | GATCTGGTGGTGTGGCTGGTGGAGATAGCGTCTGG |
| | GGACACAGTTCACTCTCACCATC | GGCCCAGGCACCCTGGTCACCGTCTCCTTAGGGCA |
| | AGCGATGTGGTGTGTGACGATGC | ACCTAAGGCTCCATCAGTCTTCCCACTGGCCCCCT |
| | TGCCACTTACTACTGTGCAGGAT | GCTGCGGGGACACACCCAGCTCCACGGTGACCTTG |
| | ATAAAAGTAGTACTACTGATGGG | GGCTGCCTGGTCAAAGGCTACCTCCCGGAGCCAGT |
| | TTTGGTTTCGGCGGAGGGACCGA | GACCGTGACCTGGAACTCGGGCACCCTCACCAATG |
| | GGTGGTGGTCAAAGGTGATCCAG | GGGTACGCACCTTCCCGTCCGTCCGGCAGTCCTCA |
| | TTGCACCTACTGTCCTCCTCTTCC | GGCCTCTACTCGCTGAGCAGCGTGGTGAGCGTGAC |
| | CACCATCTAGCGATGAGGTGGCA | CTCAAGCAGCCAGCCCGTCACCTGCAACGTGGCCC |
| | ACTGGAACAGTCACCATCGTGTG | ACCCAGCCACCAACACCAAAGTGGACAAGACCGT |
| | TGTGGCGAATAAATACTTTCCCG | TGCGCCCTCGACATGCAGCAAGCCCACGTGCCCAC |
| | ATGTCACCGTCACCTGGGAGGTG | CCCCTGAACTCCTGGGGGGACCGTCTGTCTTCATC |
| | GATGGCACCACCCAAACAACTGG | TTCCCCCCAAAACCCAAGGACACCCTCATGATCTC |
| | CATCGAGAACAGTAAAACACCGC | ACGCACCCCCGAGGTCACATGCGTGGTGGTGGACG |
| | AGAATTCTGCAGATTGTACCTAC | TGAGCCAGGATGACCCCGAGGTGCAGTTCACATGG |
| | AACCTCAGCAGCACTCTGACACT | TACATAAACAACGAGCAGGTGCGCACCGCCCGGC |
| | GACCAGCACACAGTACAACAGCC | CGCCGCTACGGGAGCAGCAGTTCAACAGCACGAT |
| | ACAAAGAGTACACCTGCAAGGTG | CCGCGTGGTCAGCACCCTCCCCATCGCGCACCAGG |
| | ACCCAGGGCACGACCTCAGTCGT | ACTGGCTGAGGGGCAAGGAGTTCAAGTGCAAAGT |
| | CCAGAGCTTCAGTAGGAAGAACT | CCACAACAAGGCACTCCCGGCCCCCATCGAGAAA |
| | GT (SEQ ID NO: 164) | ACCATCTCCAAAGCCAGAGGGCAGCCCCTGGAGC |
| | | CGAAGGTCTACACCATGGGCCCTCCCCGGGAGGA |
| | | GCTGAGCAGCAGGTCGGTCAGCCTGACCTGCATGA |
| | | TCAACGGCTTCTACCCTTCCGACATCTCGGTGGAG |
| | | TGGGAGAAGAACGGGAAGGCAGAGGACAACTACA |
| | | AGACCACGCCGGCCGTGCTGGACAGCGACGGCTC |
| | | CTACTTCCTCTACAGCAAGCTCTCAGTGCCCACGA |
| | | GTGAGTGGCAGCGGGGCGACGTCTTCACCTGCTCC |
| | | GTGATGCACGAGGCCTTGCACAACCACTACACGCA |
| | | GAAGTCCATCT CCCGCTCTCCGGGTAAA |
| | | (SEQ ID NO: 177) |
| 16A22 | GCGCAAGTGCTGGCCCAGACTCC | CAGTCACTGGAGGAGTCCGGGGGTCGCCTGGTCAC |
| | ATCCTCCGTGTCTGCAGCTGTGG | GCCTGGAGGATACCTGACACTCACCTGCACAGTCT |
| | GAGGCACAGTCACCATCGATTGC | CTGGATTCTCCCTCAGCAGCTATGACATGGGCTGG |
| | CAGTCCAGTCAGAGTGTTGGTAA | GTCCGCCAGGCTCCAGGGAAGGGGCTGGAGTGGA |
| | TAACGCCTATTTATCCTGGTATCA | TCGGATACATTTTTATTAATGATAACACATACTAC |
| | GCAGAAACCAGGGCAGCCTCCCA | GCGACCTGGGCGAAAGGCCGATTCACCATCTCCAA |
| | AGCTCCTGATCTATTATGCATCC | GACCTCGACCACGATGGATCTGAAAATGACCAGTC |
| | ACTCTGGCATCTGGGGTCCCCTC | TGACAACCGAGGACACGGCCACCTATTTCTGTGTC |
| | CCGGTTCAGTGGCAGTGGATCTG | AGAGGATATTTTGGCGGCATGGACCCCTGGGGCCC |
| | GGACACACTTCACTCTCACCATC | AGGGACCCTCGTCACCGTCTCTTCAGGGCAACCTA |
| | AGCGGCGTGCAGTGTGACGATGC | AGGCTCCATCAGTCTTCCCACTGGCCCCCTGCTGC |
| | TGCCACTTACTACTGTCAAGCCT | GGGGACACACCCAGCTCCACGGTGACCTTGGGCTG |
| | ATTACCATGTTGGTGTTGCTGCTT | CCTGGTCAAAGGCTACCTCCCGGAGCCAGTGACCG |
| | TCGGCGGAGGGACCGAGGTGGT | TGACCTGGAACTCGGGCACCCTCACCAATGGGGTA |
| | GGTCAAAGGTGATCCAGTTGCAC | CGCACCTTCCCGTCCGTCCGGCAGTCCTCAGGCCT |
| | CTACTGTCCTCCTCTTCCCACCAT | CTACTCGCTGAGCAGCGTGGTGAGCGTGACCTCAA |
| | CTAGCGATGAGGTGGCAACTGGA | GCAGCCAGCCCGTCACCTGCAACGTGGCCCACCCA |
| | ACAGTCACCATCGTGTGTGTGGC | GCCACCAACACCAAAGTGGACAAGACCGTTGCGC |
| | GAATAAATACTTTCCCGATGTCA | CCTCGACATGCAGCAAGCCCACGTGCCCACCCCCT |
| | CCGTCACCTGGGAGGTGGATGGC | GAACTCCTGGGGGGACCGTCTGTCTTCATCTTCCC |
| | ACCACCCAAACAACTGGCATCGA | CCCAAAACCCAAGGACACCCTCATGATCTCACGCA |
| | GAACAGTAAAACACCGCAGAATT | CCCCCGAGGTCACATGCGTGGTGGTGGACGTGAGC |
| | CTGCAGATTGTACCTACAACCTC | CAGGATGACCCCGAGGTGCAGTTCACATGGTACAT |
| | AGCAGCACTCTGACACTGACCAG | AAACAACGAGCAGGTGCGCACCGCCCGGCCGCCG |
| | CACACAGTACAACAGCCACAAA | CTACGGGAGCAGCAGTTCAACAGCACGATCCGCGT |
| | GAGTACACCTGCAAGGTGACCCA | GGTCAGCACCCTCCCCATCGCGCACCAGGACTGGC |
| | GGGCACGACCTCAGTCGTCCAGA | TGAGGGGCAAGGAGTTCAAGTGCAAAGTCCACAA |
| | GCTTCAGTAGGAAGAACTGT | CAAGGCACTCCCGGCCCCCATCGAGAAAACCATCT |
| | (SEQ ID NO: 165) | CCAAAGCCAGAGGGCAGCCCCTGGAGCCGAAGGT |
| | | CTACACCATGGGCCCTCCCCGGGAGGAGCTGAGCA |
| | | GCAGGTCGGTCAGCCTGACCTGCATGATCAACGGC |
| | | TTCTACCCTTCCGACATCTCGGTGGAGTGGGAGAA |
| | | GAACGGGAAGGCAGAGGACAACTACAAGACCACG |
| | | CCGGCCGTGCTGGACAGCGACGGCTCCTACTTCCT |
| | | CTACAGCAAGCTCTCAGTGCCCACGAGTGAGTGGC |
| | | AGCGGGGCGACGTCTTCACCTGCTCCGTGATGCAC |
| | | GAGGCCTTGCACAACCACTACACGCAGAAGTCCAT |
| | | CT CCCGCTCTCCGGGTAAA |
| | | (SEQ ID NO: 178) |
| 17D13 | GACGTCGTGATGACCCAGACTCC | CAGTCGGTGGAGGAGTCCGGGGGTCGCCTGGTCAC |
| | AGCCTCCGTGTCTGAATCTGTGG | GCCTGGGGCATCCCTGACACTCACCTGCACAGTCT |
| | GAGGCACAGTCACCATCAAGTGC | CTGGAATCGACTTCAGTACCAATGCAATGACCTGG |
| | CAGGCCAGTCAGGATCTTTATAG | GTCCGCCAGGCTCCAGGGAAGGGGCTGGAATGGA |
| | TAATTGTTTATCCTGGTATCAGCA | TCGGATATATTTATGAGGGTAGTGGTAATACATTC |
| | GAAACCAGGGCAGCGTCCCAAG | TACGCGAGCTGGGCGAAAGGCCGATTCACCATTTC |
| | CTCCTGATGTATCTGACATCCACT | CAGAACCTCGACCACGGTGGATTTGAAAATGACCA |
| | CTGGCATCTGGGGTCCCATCGCG | GTCTGACAATGGAGGACACGGCCACCTATTTCTGT |
| | GTTCAAAGGCAGTGGATCTGGGA | GCCAGAGGATATCTCGGCGCCATGGACCCCTGGGG |
| | CAGATTTCACTCTCACCATCAGC | CCCAGGGACCCTCGTCACCGTCTCTTCAGGGCAAC |
| | GACCTGGAGTGTGCCGATGCTGC | CTAAGGCTCCATCAGTCTTCCCACTGGCCCCCTGCT |
| | CACTTACTACTGTCAAGGCTTCC | GCGGGGACACACCCAGCTCCACGGTGACCTTGGGC |
| | ATGGTTATGGTGTTGGTGCCGCT | TGCCTGGTCAAAGGCTACCTCCCGGAGCCAGTGAC |
| | TTCGGCGGAGGGACCGAGGTGGT | CGTGACCTGGAACTCGGGCACCCTCACCAATGGGG |
| | GGTCAAGGGTGATCCAGTTGCAC | TACGCACCTTCCCGTCCGTCCGGCAGTCCTCAGGC |
| | CTACTGTCCTCCTCTTCCCACCAT | CTCTACTCGCTGAGCAGCGTGGTGAGCGTGACCTC |
| | CTAGCGATGAGGTGGCAACTGGA | AAGCAGCCAGCCCGTCACCTGCAACGTGGCCCACC |
| | ACAGTCACCATCGTGTGTGTGGC | CAGCCACCAACACCAAAGTGGACAAGACCGTTGC |
| | GAATAAATACTTTCCCGATGTCA | GCCCTCGACATGCAGCAAGCCCACGTGCCCACCCC |
| | CCGTCACCTGGGAGGTGGATGGC | CTGAACTCCTGGGGGGACCGTCTGTCTTCATCTTCC |
| | ACCACCCAAACAACTGGCATCGA | CCCCAAAACCCAAGGACACCCTCATGATCTCACGC |
| | GAACAGTAAAACACCGCAGAATT | ACCCCCGAGGTCACATGCGTGGTGGTGGACGTGAG |
| | CTGCAGATTGTACCTACAACCTC | CCAGGATGACCCCGAGGTGCAGTTCACATGGTACA |
| | AGCAGCACTCTGACACTGACCAG | TAAACAACGAGCAGGTGCGCACCGCCCGGCCGCC |
| | CACACAGTACAACAGCCACAAA | GCTACGGGAGCAGCAGTTCAACAGCACGATCCGC |
| | GAGTACACCTGCAAGGTGACCCA | GTGGTCAGCACCCTCCCCATCGCGCACCAGGACTG |
| | GGGCACGACCTCAGTCGTCCAGA | GCTGAGGGGCAAGGAGTTCAAGTGCAAAGTCCAC |
| | GCTTCAGTAGGAAGAACTGT | AACAAGGCACTCCCGGCCCCCATCGAGAAAACCA |
| | (SEQ ID NO: 166) | TCTCCAAAGCCAGAGGGCAGCCCCTGGAGCCGAA |
| | | GGTCTACACCATGGGCCCTCCCCGGGAGGAGCTGA |
| | | GCAGCAGGTCGGTCAGCCTGACCTGCATGATCAAC |
| | | GGCTTCTACCCTTCCGACATCTCGGTGGAGTGGGA |
| | | GAAGAACGGGAAGGCAGAGGACAACTACAAGACC |
| | | ACGCCGGCCGTGCTGGACAGCGACGGCTCCTACTT |
| | | CCTCTACAGCAAGCTCTCAGTGCCCACGAGTGAGT |
| | | GGCAGCGGGGCGACGTCTTCACCTGCTCCGTGATG |
| | | CACGAGGCCTTGCACAACCACTACACGCAGAAGTC |
| | | CATCT CCCGCTCTCCGGGTAAA |
| | | (SEQ ID NO: 179) |
| 18J5 | GACGTCGTGATGACCCAGACTCC | CAGTCAGTGGAGGAGTCCGGGGGAGGCCTGGTCA |
| | AGCCTCCGTGGAGGCAGCTGTGG | AGCCTGGGGCATCCCTGACACTCACCTGTCAAGTC |
| | GAGGCACAGTCACCATCAAGTGC | TCTGGATTCTCCCTCAGTGACCACTACATGAGCTG |
| | CAGGCCAGTCAGAGCATTAATAG | GGTCCGCCAGGCTCCAGGGAAGGGGCTGGAATGG |
| | TTGGTTATCCTGGTATCAGCAGA | GTCGCATATATTAGTGAGGGTGGTGCCACATACTA |
| | AACCAGGGCAGCGTCCCAAACTC | CGCGAGCTGGGCAAAAGGCCGATTCACCATCTCCA |
| | CTGATCTATGCTGCATCCACTCTG | AAACCTCGTCGACCACGGTGGATCTGAAAATGACC |
| | GCATCTGGGGTCTCATCGCGGTT | AGTCTGACAACCGAGGACACGGCCACCTATTTTTG |
| | CAAAGGCAGTAAATCTGGGACA | TGCCAGAGGATGGCTTGCTGCTTTTGATCCCTGGG |
| | GAGTTCACTCTCACCATCAGCGG | GCCCAGGCACCCTGGTCACCGTCTCCTCAGGGCAA |
| | TGTGCAGTGTGACGATGCTGCCA | CCTAAGGCTCCATCAGTCTTCCCACTGGCCCCCTG |
| | CTTATTATTGTCAAGGCTATGAT | CTGCGGGGACACACCCAGCTCCACGGTGACCTTGG |
| | GGTAGTAGTGGTAGTGCTGCTAG | GCTGCCTGGTCAAAGGCTACCTCCCGGAGCCAGTG |
| | TTTCGGCGGAGGGACCGAGGTGG | ACCGTGACCTGGAACTCGGGCACCCTCACCAATGG |
| | TGGTCAAAGGTGATCCAGTTGCA | GGTACGCACCTTCCCGTCCGTCCGGCAGTCCTCAG |
| | CCTACTGTCCTCCTCTTCCCACCA | GCCTCTACTCGCTGAGCAGCGTGGTGAGCGTGACC |
| | TCTAGCGATGAGGTGGCAACTGG | TCAAGCAGCCAGCCCGTCACCTGCAACGTGGCCCA |
| | AACAGTCACCATCGTGTGTGTGG | CCCAGCCACCAACACCAAAGTGGACAAGACCGTT |
| | CGAATAAATACTTTCCCGATGTC | GCGCCCTCGACATGCAGCAAGCCCACGTGCCCACC |
| | ACCGTCACCTGGGAGGTGGATGG | CCCTGAACTCCTGGGGGGACCGTCTGTCTTCATCTT |
| | CACCACCCAAACAACTGGCATCG | CCCCCCAAAACCCAAGGACACCCTCATGATCTCAC |
| | AGAACAGTAAAACACCGCAGAA | GCACCCCCGAGGTCACATGCGTGGTGGTGGACGTG |
| | TTCTGCAGATTGTACCTACAACC | AGCCAGGATGACCCCGAGGTGCAGTTCACATGGTA |
| | TCAGCAGCACTCTGACACTGACC | CATAAACAACGAGCAGGTGCGCACCGCCCGGCCG |
| | AGCACACAGTACAACAGCCACA | CCGCTACGGGAGCAGCAGTTCAACAGCACGATCC |
| | AAGAGTACACCTGCAAGGTGACC | GCGTGGTCAGCACCCTCCCCATCGCGCACCAGGAC |
| | CAGGGCACGACCTCAGTCGTCCA | TGGCTGAGGGGCAAGGAGTTCAAGTGCAAAGTCC |
| | GAGCTTCAGTAGGAAGAACTGT | ACAACAAGGCACTCCCGGCCCCCATCGAGAAAAC |
| | (SEQ ID NO: 167) | CATCTCCAAAGCCAGAGGGCAGCCCCTGGAGCCG |
| | | AAGGTCTACACCATGGGCCCTCCCCGGGAGGAGCT |
| | | GAGCAGCAGGTCGGTCAGCCTGACCTGCATGATCA |
| | | ACGGCTTCTACCCTTCCGACATCTCGGTGGAGTGG |
| | | GAGAAGAACGGGAAGGCAGAGGACAACTACAAG |
| | | ACCACGCCGGCCGTGCTGGACAGCGACGGCTCCTA |
| | | CTTCCTCTACAGCAAGCTCTCAGTGCCCACGAGTG |
| | | AGTGGCAGCGGGGCGACGTCTTCACCTGCTCCGTG |
| | | ATGCACGAGGCCTTGCACAACCACTACACGCAGA |
| | | AGTCCATCT CCCGCTCTCCGGGTAAA |
| | | (SEQ ID NO: 180) |

The nucleic acid sequences provided in Tables 3 and 4 are exemplary only. As will be appreciated by those in the art, due to the degeneracy of the genetic code, an extremely large number of nucleic acids may be made, all of which encode the CDRs, variable regions, and heavy and light chains of the antibodies described herein. Thus, having identified a particular amino acid sequence, those skilled in the art could make any number of different nucleic acids, by modifying the sequence of one or more codons in a way which does not change the amino acid sequence of the encoded protein. Accordingly, isolated polynucleotides encoding the antibodies and antigen-binding fragments of the invention may comprise a nucleotide sequence that is at least 80% identical, at least 90% identical, at least 95% identical, or at least 98% identical to any of the nucleotide sequences listed in Tables 3 and 4.

In some embodiments, an isolated polynucleotide encoding a pan-specific antibody light chain variable region comprises a sequence that is at least 80% identical, at least 90% identical, at least 95% identical, or at least 98% identical to a sequence selected from SEQ ID NOs: 129 to 131. In other embodiments, an isolated polynucleotide encoding a pan-specific antibody light chain variable region comprises a sequence selected from SEQ ID NOs: 129 to 131. In certain embodiments, an isolated polynucleotide encoding a pan-specific antibody light chain comprises a sequence that is at least 80% identical, at least 90% identical, at least 95% identical, or at least 98% identical to a sequence selected from SEQ ID NOs: 155 to 157. In certain other embodiments, an isolated polynucleotide encoding a pan-specific antibody light chain comprises a sequence selected from SEQ ID NOs: 155 to 157. In these and other embodiments, an isolated polynucleotide encoding a pan-specific antibody heavy chain variable region comprises a sequence that is at least 80% identical, at least 90% identical, at least 95% identical, or at least 98% identical to a sequence selected from SEQ ID NOs: 142 to 144. In some embodiments, an isolated polynucleotide encoding a pan-specific antibody heavy chain variable region comprises a sequence selected from SEQ ID NOs: 142 to 144. In related embodiments, an isolated polynucleotide encoding a pan-specific antibody heavy chain comprises a sequence that is at least 80% identical, at least 90% identical, at least 95% identical, or at least 98% identical to a sequence selected from SEQ ID NOs: 168 to 170. In some embodiments, an isolated polynucleotide encoding a pan-specific antibody heavy chain comprises a sequence selected from SEQ ID NOs: 168 to 170.

In certain embodiments, an isolated polynucleotide encoding an 1851 RNAi construct-specific antibody light chain variable region comprises a sequence that is at least 80% identical, at least 90% identical, at least 95% identical, or at least 98% identical to a sequence selected from SEQ ID NOs: 132 to 136. In certain other embodiments, an isolated polynucleotide encoding an 1851 RNAi construct-specific antibody light chain variable region comprises a sequence selected from SEQ ID NOs: 132 to 136. In some embodiments, an isolated polynucleotide encoding an 1851 RNAi construct-specific antibody light chain comprises a sequence that is at least 80% identical, at least 90% identical, at least 95% identical, or at least 98% identical to a sequence selected from SEQ ID NOs: 158 to 162. In other embodiments, an isolated polynucleotide encoding an 1851 RNAi construct-specific antibody light chain comprises a sequence selected from SEQ ID NOs: 158 to 162. In these and other embodiments, an isolated polynucleotide encoding an 1851 RNAi construct-specific antibody heavy chain variable region comprises a sequence that is at least 80% identical, at least 90% identical, at least 95% identical, or at least 98% identical to a sequence selected from SEQ ID NOs: 145 to 149. In some embodiments, an isolated polynucleotide encoding an 1851 RNAi construct-specific antibody heavy chain variable region comprises a sequence selected from SEQ ID NOs: 145 to 149. In related embodiments, an isolated polynucleotide encoding an 1851 RNAi construct-specific antibody heavy chain comprises a sequence that is at least 80% identical, at least 90% identical, at least 95% identical, or at least 98% identical to a sequence selected from SEQ ID NOs: 171 to 175. In some embodiments, an isolated polynucleotide encoding an 1851 RNAi construct-specific antibody heavy chain comprises a sequence selected from SEQ ID NOs: 171 to 175.

In certain other embodiments, an isolated polynucleotide encoding a GalNAc moiety-specific antibody light chain variable region comprises a sequence that is at least 80% identical, at least 90% identical, at least 95% identical, or at least 98% identical to a sequence selected from SEQ ID NOs: 137 to 141. In some embodiments, an isolated polynucleotide encoding a GalNAc moiety-specific antibody light chain variable region comprises a sequence selected from SEQ ID NOs: 137 to 141. In other embodiments, an isolated polynucleotide encoding a GalNAc moiety-specific antibody light chain comprises a sequence that is at least 80% identical, at least 90% identical, at least 95% identical, or at least 98% identical to a sequence selected from SEQ ID NOs: 163 to 167. In yet other embodiments, an isolated polynucleotide encoding a GalNAc moiety-specific antibody light chain comprises a sequence selected from SEQ ID NOs: 163 to 167. In these and other embodiments, an isolated polynucleotide encoding a GalNAc moiety-specific antibody heavy chain variable region comprises a sequence that is at least 80% identical, at least 90% identical, at least 95% identical, or at least 98% identical to a sequence selected from SEQ ID NOs: 150 to 154. In some embodiments, an isolated polynucleotide encoding a GalNAc moiety-specific antibody heavy chain variable region comprises a sequence selected from SEQ ID NOs: 150 to 154. In related embodiments, an isolated polynucleotide encoding a GalNAc moiety-specific antibody heavy chain comprises a sequence that is at least 80% identical, at least 90% identical, at least 95% identical, or at least 98% identical to a sequence selected from SEQ ID NOs: 176 to 180. In other related embodiments, an isolated polynucleotide encoding a GalNAc moiety-specific antibody heavy chain comprises a sequence selected from SEQ ID NOs: 176 to 180.

Expression vectors comprising one or more polynucleotides encoding the antibodies of the invention or portions thereof can be constructed from the polynucleotide sequences described above and used to transform host cells to produce the antibodies or antigen-binding fragments of the invention. The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant nucleic acid molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Typically, expression vectors used in the host cells to produce antibodies and antigen-binding fragments of the invention will contain sequences for cloning and expression of exogenous nucleotide sequences encoding the components of the antibodies or antigen-binding fragments. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Expression and cloning vectors will typically contain a promoter that is recognized by the host cell and operably linked to the nucleic acid molecule encoding the polypeptide. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system. A large number of promoters, recognized by a variety of potential host cells, are well known. A suitable promoter is operably linked to the polynucleotide encoding e.g., heavy chain, light chain, or other component of the antibodies or antigen-binding fragments of the invention, by removing the promoter from the source nucleic acid by restriction enzyme digestion and inserting the desired promoter sequence into the vector.

The expression vectors may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art. The expression vectors can be introduced into host cells to thereby produce the antibodies and antigen-binding fragments encoded by the nucleic acids present in the vectors.

After the vector has been constructed and the one or more nucleic acid molecules encoding the components of the antibodies and antigen-binding fragments described herein has been inserted into the proper site(s) of the vector or vectors, the completed vector(s) may be inserted into a suitable host cell for amplification and/or polypeptide expression. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated polynucleotide of the invention, preferably operably linked to at least one expression control sequence (e.g. promoter or enhancer), is a “recombinant host cell.”

The transformation of an expression vector for an antibody or antigen-binding fragment of the invention into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

A host cell, when cultured under appropriate conditions, synthesizes an antibody, antigen-binding fragment, or antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. Suitable host cells include, but are not limited to, prokaryotic cells (e.g. E. coli, B. subtilis), yeast cells (Saccharmoyces cerevisiae, Pichia pastoris), and mammalian cells (e.g. Chinese hamster ovary (CHO), human embryonic kidney (HEK)).

The host cells used to produce the antibodies and antigen-binding fragments of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinary skilled artisan.

Upon culturing the host cells, the antibody or antigen-binding fragment can be produced intracellularly, in the periplasmic space, or directly secreted into the medium. If the antibody or antigen-binding fragment is produced intracellularly, as a first step, the particulate debris, either host cells or lysed fragments, is removed, for example, by centrifugation or ultrafiltration. The antibody or antigen-binding fragment can be purified from culture medium, culture supernatant or other fluid following a harvest step using, for example, hydroxyapatite chromatography, cation or anion exchange chromatography, or preferably affinity chromatography, using the antigen(s) of interest or protein A or protein G as an affinity ligand.

In certain embodiments, the antibodies or antigen-binding fragments of the invention are used in diagnostic or analyte detection methods, such as those described herein. Accordingly, in some embodiments, the antibodies or antigen-binding fragments of the invention are coupled to a detectable label. The detectable label can be any molecular entity that is capable of producing a detectable signal under a particular set of conditions. Conventional labels may be used which are capable, alone or in concert with other compositions or compounds, of providing a detectable signal. The detectable label can be a radiolabel, an enzyme, a fluorophore, a chromophore, a chemiluminescent label, an electrochemiluminescence (ECL) luminophore, a metallic nanoparticle, or a metallic nanoshell.

In one embodiment, the detectable label coupled to the binding partner is a metallic nanoparticle or metallic nanoshell. Suitable metallic nanoparticles or nanoshells for use as the detectable label include, but are not limited to, gold nanoparticles, silver nanoparticles, copper nanoparticles, platinum nanoparticles, cadmium nanoparticles, composite nanoparticles (e.g. silver and gold or copper and silver), gold hollow spheres, gold-coated silica nanoshells, and silica-coated gold shells. In another embodiment, the detectable label coupled to the binding partner is an enzyme that can convert a substrate into a detectable signal, e.g. a colored, fluorescent, or chemiluminescent product. Non-limiting examples of enzymes that are suitable for coupling to an antibody or antigen-binding fragment of the invention include alkaline phosphatase, horseradish peroxidase, beta-galactosidase, beta-lactamase, galactose oxidase, lactoperoxidase, luciferase, myeloperoxidase, and amylase. In another embodiment, the detectable label coupled to an antibody or antigen-binding fragment of the invention is a fluorophore. Exemplary fluorescent molecules suitable for use as detectable labels include fluorescein, Texas-Red, green fluorescent protein, yellow fluorescent protein, cyan fluorescent protein, Alexa dye molecules, rhodamine dye molecules, and the like. In yet another embodiment, the detectable label coupled to an antibody or antigen-binding fragment of the invention is a radiolabel. Suitable radiolabels include, but are not limited to, ¹²⁵I, ¹³¹I, ³H, ¹⁴C, ¹³N, ¹⁸F, and ³⁵S.

In certain embodiments, the detectable label coupled to an antibody or antigen-binding fragment of the invention is an ECL luminophore. ECL luminophores that can be coupled to an antibody or antigen-binding fragment of the invention include, but are not limited to, ruthenium complexes (e.g. tri-2,2′-bipyridylruthenium(II) [Ru(bpy)₃²⁺]), iridium complexes, aluminum complexes, chromium complexes, copper complexes, europium complexes, osmium complexes, platinum complexes, and rhenium complexes, such as those described in Richter, Chem. Rev., Vol. 104: 3003-3036, 2004, Liu et al., Chem. Soc. Rev., Vol. 44, 3117-3142, 2015, and Zhou et al., Dalton Trans., Vol. 46, 355-363, 2017. In certain embodiments, the ECL luminophore coupled to the antibody or antigen-binding fragment of the invention is a ruthenium complex.

Methods of coupling the detectable label to proteins, such as the antibodies or antigen-binding fragments of the invention, are known in the art and can include passive adsorption (e.g. when metallic nanoparticles or nanoshells are the detectable label) and conjugation chemistries, such as succinimide ester coupling to primary amines and maleimide coupling to sulfhydryl groups. Other methods of coupling macromolecules to detectable labels are known to the skilled artisan, who can select the proper method based on the type of desired detectable label to be used.

Any of the antibodies or antigen-binding fragments described herein or generated by the methods of the invention can be incorporated into immunoassays to detect chemically-modified nucleic acid molecules in various samples, including biological samples. Accordingly, the present invention provides methods for detecting a chemically-modified nucleic acid molecule in a sample. In one embodiment, the methods comprise: providing a surface comprising a capture antibody or antigen-binding fragment thereof that specifically binds to the chemically-modified nucleic acid molecule; contacting the surface with a sample under conditions allowing the chemically-modified nucleic acid molecule, if present in the sample, to bind to the capture antibody or antigen-binding fragment thereof on the surface; contacting the surface with a detection reagent, wherein the detection reagent comprises a detectable label coupled to a binding partner that specifically binds to the chemically-modified nucleic acid molecule; and detecting a signal from the detectable label.

In certain embodiments of the detection methods of the invention, the capture antibody that specifically binds to the chemically-modified nucleic acid molecule is one of the pan-specific antibodies described herein, for example, the 14K10, 14F4, or 5117 antibody. In one embodiment, the capture antibody comprises a CDRL1 of SEQ ID NO: 1, a CDRL2 of SEQ ID NO: 14, a CDRL3 of SEQ ID NO: 25, a CDRH1 of SEQ ID NO: 51, a CDRH2 of SEQ ID NO: 64, and a CDRH3 of SEQ ID NO: 77. In another embodiment, the capture antibody comprises a CDRL1 of SEQ ID NO: 2, a CDRL2 of SEQ ID NO: 15, a CDRL3 of SEQ ID NO: 26, a CDRH1 of SEQ ID NO: 52, a CDRH2 of SEQ ID NO: 65, and a CDRH3 of SEQ ID NO: 78. In another embodiment, the capture antibody comprises a CDRL1 of SEQ ID NO: 3, a CDRL2 of SEQ ID NO: 16, a CDRL3 of SEQ ID NO: 27, a CDRH1 of SEQ ID NO: 53, a CDRH2 of SEQ ID NO: 66, and a CDRH3 of SEQ ID NO: 79. In some embodiments, the capture antibody comprises: (a) a light chain variable region comprising the sequence of SEQ ID NO: 38 and a heavy chain variable region comprising the sequence of SEQ ID NO: 90; (b) a light chain variable region comprising the sequence of SEQ ID NO: 39 and a heavy chain variable region comprising the sequence of SEQ ID NO: 91; or (c) a light chain variable region comprising the sequence of SEQ ID NO: 40 and a heavy chain variable region comprising the sequence of SEQ ID NO: 92. In other embodiments, the capture antibody comprises: (a) a light chain comprising the sequence of SEQ ID NO: 103 and a heavy chain comprising the sequence of SEQ ID NO: 116; (b) a light chain comprising the sequence of SEQ ID NO: 104 and a heavy chain comprising the sequence of SEQ ID NO: 117; or (c) a light chain comprising the sequence of SEQ ID NO: 105 and a heavy chain comprising the sequence of SEQ ID NO: 118.

In some embodiments of the detection methods of the invention in which the chemically-modified nucleic acid molecule to be detected is the 1851 RNAi construct, the capture antibody can be any of the 1851 RNAi construct-specific antibodies described herein, for example, the 17K13, 17F22, 20K24, 20P19, or 19F24 antibody. In one such embodiment, the capture antibody comprises a CDRL1 of SEQ ID NO: 4, a CDRL2 of SEQ ID NO: 17, a CDRL3 of SEQ ID NO: 28, a CDRH1 of SEQ ID NO: 54, a CDRH2 of SEQ ID NO: 67, and a CDRH3 of SEQ ID NO: 80. In another embodiment, the capture antibody comprises a CDRL1 of SEQ ID NO: 5, a CDRL2 of SEQ ID NO: 18, a CDRL3 of SEQ ID NO: 29, a CDRH1 of SEQ ID NO: 55, a CDRH2 of SEQ ID NO: 68, and a CDRH3 of SEQ ID NO: 81. In another embodiment, the capture antibody comprises a CDRL1 of SEQ ID NO: 6, a CDRL2 of SEQ ID NO: 19, a CDRL3 of SEQ ID NO: 30, a CDRH1 of SEQ ID NO: 56, a CDRH2 of SEQ ID NO: 69, and a CDRH3 of SEQ ID NO: 82. In yet another embodiment, the capture antibody comprises a CDRL1 of SEQ ID NO: 7, a CDRL2 of SEQ ID NO: 20, a CDRL3 of SEQ ID NO: 31, a CDRH1 of SEQ ID NO: 57, a CDRH2 of SEQ ID NO: 70, and a CDRH3 of SEQ ID NO: 83. In still another embodiment, the capture antibody comprises a CDRL1 of SEQ ID NO: 8, a CDRL2 of SEQ ID NO: 17, a CDRL3 of SEQ ID NO: 32, a CDRH1 of SEQ ID NO: 58, a CDRH2 of SEQ ID NO: 71, and a CDRH3 of SEQ ID NO: 84.

In some embodiments of the detection methods, the capture antibody comprises: (a) a light chain variable region comprising the sequence of SEQ ID NO: 41 and a heavy chain variable region comprising the sequence of SEQ ID NO: 93; (b) a light chain variable region comprising the sequence of SEQ ID NO: 42 and a heavy chain variable region comprising the sequence of SEQ ID NO: 94; (c) a light chain variable region comprising the sequence of SEQ ID NO: 43 and a heavy chain variable region comprising the sequence of SEQ ID NO: 95; (d) a light chain variable region comprising the sequence of SEQ ID NO: 44 and a heavy chain variable region comprising the sequence of SEQ ID NO: 96; or (e) a light chain variable region comprising the sequence of SEQ ID NO: 45 and a heavy chain variable region comprising the sequence of SEQ ID NO: 97. In other embodiments of the detection methods of the invention, the capture antibody comprises: (a) a light chain comprising the sequence of SEQ ID NO: 106 and a heavy chain comprising the sequence of SEQ ID NO: 119; (b) a light chain comprising the sequence of SEQ ID NO: 107 and a heavy chain comprising the sequence of SEQ ID NO: 120; (c) a light chain comprising the sequence of SEQ ID NO: 108 and a heavy chain comprising the sequence of SEQ ID NO: 121; (d) a light chain comprising the sequence of SEQ ID NO: 109 and a heavy chain comprising the sequence of SEQ ID NO: 122; or (e) a light chain comprising the sequence of SEQ ID NO: 110 and a heavy chain comprising the sequence of SEQ ID NO: 123.

The capture antibodies employed in the methods of the invention are preferably attached to or immobilized on a surface. The surface can be a bead or particle (e.g. a magnetic bead or particle comprising silica, latex, polystyrene, polycarbonate, polyacrylate, or polyvinylidene fluoride (PVDF)), a membrane (e.g. PVDF, nitrocellulose, polyethylene, or nylon membrane), a tube, a resin, a column, an electrode, or a well in an assay plate (e.g. a well in a microtiter plate). Such surfaces can comprise glass, cellulose-based materials, thermoplastic polymers, such as polyethylene, polypropylene, or polyester, sintered structures composed of particulate materials (e.g., glass or various thermoplastic polymers), or cast membrane film composed of nitrocellulose, nylon, polysulfone, or the like. All of these surface materials can be used in suitable shapes, such as films, sheets, or plates, or they may be coated onto or bonded or laminated to appropriate inert carriers, such as paper, glass, plastic films, or fabrics.

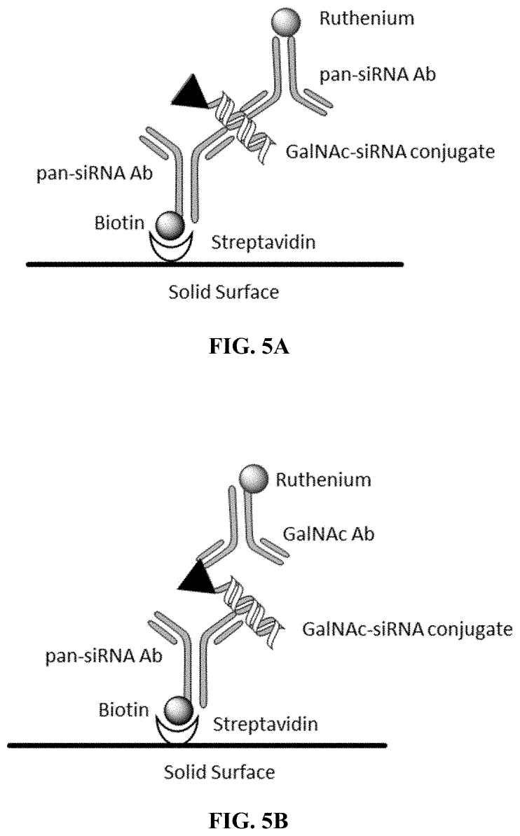

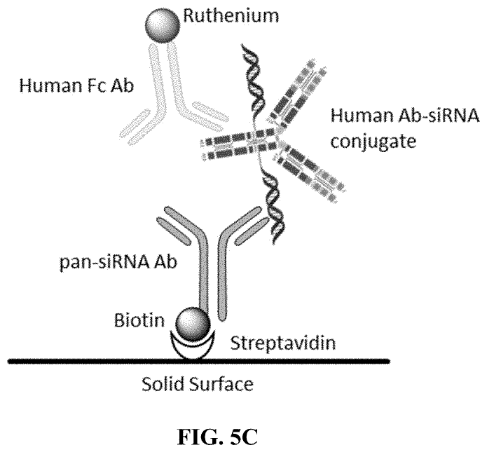

The capture antibodies can be immobilized on or attached to a surface by a variety of procedures known to those of skill in the art. The capture antibodies can be striped, deposited, or printed on the surface followed by drying of the surface to facilitate immobilization. Immobilization of the capture antibodies can take place through adsorption or covalent bonding. Depending on the nature of the surface, methods of derivatization to facilitate the formation of covalent bonds between the surface and the capture antibody can be used. Methods of derivatization can include treating the surface with a compound, such as glutaraldehyde or carbodiimide, and applying the capture antibody. The capture antibody can also be attached to the surface indirectly through a moiety coupled to the capture antibody that enables covalent or non-covalent binding, such as a moiety that has a high affinity to a component attached to the surface. For example, the capture antibody can be coupled to biotin, and the component attached to the surface can be avidin, streptavidin, or neutravidin (see, e.g. FIG. 5A to FIG. 5C). Other physical, chemical, or biological methods of immobilizing an antibody either directly or indirectly to a surface are known in the art and can be used to immobilize or attach the capture antibody to a surface.

Following contact of a sample with the surface comprising a capture antibody and any optional wash steps to remove unbound molecules, the detection methods of the invention comprise contacting the surface with a detection reagent and detecting a signal from a detectable label in the detection reagent. The detection reagent comprises a detectable label coupled to a binding partner that specifically binds to the chemically-modified nucleic acid molecule. The binding partner in the detection reagent can be an antibody or antigen-binding fragment thereof, an aptamer, a polynucleotide that hybridizes to the chemically-modified nucleic acid molecule, or other molecule that is able to specifically bind to the chemically-modified nucleic acid molecule.

In certain embodiments, the binding partner in the detection reagent is an antibody or antigen-binding fragment that specifically binds to the chemically-modified nucleic acid molecule. In some such embodiments, the antibody in the detection reagent is a polyclonal antibody. Polyclonal antibodies that bind to the chemically-modified nucleic acid molecule of interest can be generated by immunizing an immunocompetent animal (e.g. mouse, rabbit, rat, goat, or other mammal) with the chemically-modified nucleic acid molecule coupled to a carrier protein such as bovine serum albumin or keyhole limpet hemocyanin in the absence or presence of an adjuvant. In other embodiments, the antibody in the detection reagent is one of the pan-specific antibodies described herein, for example, the 14K10, 14F4, or 5117 antibody. In some such embodiments, both the capture antibody immobilized to a surface and the antibody in the detection reagent are pan-specific antibodies described herein. See, e.g., FIG. 5A. In such embodiments, the capture antibody and the antibody in the detection reagent can be the same pan-specific antibody (e.g. 14K10 antibody) or they can be different antibodies. In one particular embodiment, the antibody in the detection reagent comprises a CDRL1 of SEQ ID NO: 1, a CDRL2 of SEQ ID NO: 14, a CDRL3 of SEQ ID NO: 25, a CDRH1 of SEQ ID NO: 51, a CDRH2 of SEQ ID NO: 64, and a CDRH3 of SEQ ID NO: 77. In another particular embodiment, the antibody in the detection reagent comprises a light chain variable region comprising the sequence of SEQ ID NO: 38 and a heavy chain variable region comprising the sequence of SEQ ID NO: 90. In yet another particular embodiment, the antibody in the detection reagent comprises a light chain comprising the sequence of SEQ ID NO: 103 and a heavy chain comprising the sequence of SEQ ID NO: 116.

In embodiments of the methods of the invention in which the chemically-modified nucleic acid molecule to be detected is the 1851 RNAi construct, the antibody in the detection reagent can be any of the 1851 RNAi construct-specific antibodies described herein, for example, the 17K13, 17F22, 20K24, 20P19, or 19F24 antibody. In one such embodiment, the antibody in the detection reagent comprises a CDRL1 of SEQ ID NO: 4, a CDRL2 of SEQ ID NO: 17, a CDRL3 of SEQ ID NO: 28, a CDRH1 of SEQ ID NO: 54, a CDRH2 of SEQ ID NO: 67, and a CDRH3 of SEQ ID NO: 80. In another embodiment, the antibody in the detection reagent comprises a CDRL1 of SEQ ID NO: 5, a CDRL2 of SEQ ID NO: 18, a CDRL3 of SEQ ID NO: 29, a CDRH1 of SEQ ID NO: 55, a CDRH2 of SEQ ID NO: 68, and a CDRH3 of SEQ ID NO: 81. In another embodiment, the antibody in the detection reagent comprises a CDRL1 of SEQ ID NO: 6, a CDRL2 of SEQ ID NO: 19, a CDRL3 of SEQ ID NO: 30, a CDRH1 of SEQ ID NO: 56, a CDRH2 of SEQ ID NO: 69, and a CDRH3 of SEQ ID NO: 82. In some embodiments of the detection methods, the antibody in the detection reagent comprises: (a) a light chain variable region comprising the sequence of SEQ ID NO: 41 and a heavy chain variable region comprising the sequence of SEQ ID NO: 93; (b) a light chain variable region comprising the sequence of SEQ ID NO: 42 and a heavy chain variable region comprising the sequence of SEQ ID NO: 94; or (c) a light chain variable region comprising the sequence of SEQ ID NO: 43 and a heavy chain variable region comprising the sequence of SEQ ID NO: 95. In other embodiments of the detection methods of the invention, the antibody in the detection reagent comprises: (a) a light chain comprising the sequence of SEQ ID NO: 106 and a heavy chain comprising the sequence of SEQ ID NO: 119; (b) a light chain comprising the sequence of SEQ ID NO: 107 and a heavy chain comprising the sequence of SEQ ID NO: 120; or (c) a light chain comprising the sequence of SEQ ID NO: 108 and a heavy chain comprising the sequence of SEQ ID NO: 121.

For the detection of the 1851 RNAi construct specifically in biological samples, any of the 1851 RNAi construct-specific antibodies can be used as the capture antibody or the antibody in the detection reagent or both. For instance, in some embodiments of the detection methods of the invention, a pan-specific antibody described herein (e.g. 14K10 antibody) is used as the capture antibody and an 1851 RNAi construct-specific antibody described herein (e.g. 17K13, 17F22, or 20K24 antibody) is used as the antibody in the detection reagent. In other embodiments of the detection methods of the invention, an 1851 RNAi construct-specific antibody described herein (e.g. 17K13, 17F22, or 20K24 antibody) is used as the capture antibody and a pan-specific antibody described herein (e.g. 14K10 antibody) is used as the antibody in the detection reagent. In certain embodiments of the detection methods of the invention, an 1851 RNAi construct-specific antibody described herein is used both as the capture antibody and the antibody in the detection reagent. In such embodiments, the same 1851 RNAi construct-specific antibody can be used as both the capture antibody and antibody in the detection reagent. Alternatively, different antibodies can be used as the capture antibody and antibody in the detection reagent, e.g. 17K13 antibody as capture antibody and 20K24 antibody as the antibody in the detection reagent.

In certain embodiments, the chemically-modified nucleic acid molecule to be detected is covalently linked to a ligand comprising a GalNAc moiety, such as any of the GalNAc moieties described herein. In such embodiments, the binding partner in the detection reagent can be a molecule that specifically binds GalNAc residues, such as a lectin, ligand binding domain of an ASGR receptor, or an antibody or antigen-binding fragment thereof. In particular embodiments, the binding partner in the detection reagent is one of the GalNAc moiety-specific antibodies described herein, for example, the 14D4, 1613, 16A22, 17D13, or 18J5 antibody. In some such embodiments, the capture antibody immobilized to a surface is one of the pan-specific antibodies described herein (e.g. 14K10 antibody) and the antibody in the detection reagent is one of the GalNAc moiety-specific antibodies described herein (e.g. 14D4 antibody). See, e.g., FIG. 5B. In one such embodiment, the antibody in the detection reagent comprises a CDRL1 of SEQ ID NO: 9, a CDRL2 of SEQ ID NO: 21, a CDRL3 of SEQ ID NO: 33, a CDRH1 of SEQ ID NO: 59, a CDRH2 of SEQ ID NO: 72, and a CDRH3 of SEQ ID NO: 85. In another embodiment, the antibody in the detection reagent comprises a CDRL1 of SEQ ID NO: 10, a CDRL2 of SEQ ID NO: 22, a CDRL3 of SEQ ID NO: 34, a CDRH1 of SEQ ID NO: 60, a CDRH2 of SEQ ID NO: 73, and a CDRH3 of SEQ ID NO: 86. In another embodiment, the antibody in the detection reagent comprises a CDRL1 of SEQ ID NO: 11, a CDRL2 of SEQ ID NO: 19, a CDRL3 of SEQ ID NO: 35, a CDRH1 of SEQ ID NO: 61, a CDRH2 of SEQ ID NO: 74, and a CDRH3 of SEQ ID NO: 87. In yet another embodiment, the antibody in the detection reagent comprises a CDRL1 of SEQ ID NO: 12, a CDRL2 of SEQ ID NO: 23, a CDRL3 of SEQ ID NO: 36, a CDRH1 of SEQ ID NO: 62, a CDRH2 of SEQ ID NO: 75, and a CDRH3 of SEQ ID NO: 88. In still another embodiment, the antibody in the detection reagent comprises a CDRL1 of SEQ ID NO: 13, a CDRL2 of SEQ ID NO: 24, a CDRL3 of SEQ ID NO: 37, a CDRH1 of SEQ ID NO: 63, a CDRH2 of SEQ ID NO: 76, and a CDRH3 of SEQ ID NO: 89.

In some embodiments of the detection methods, the antibody in the detection reagent comprises: (a) a light chain variable region comprising the sequence of SEQ ID NO: 46 and a heavy chain variable region comprising the sequence of SEQ ID NO: 98; (b) a light chain variable region comprising the sequence of SEQ ID NO: 47 and a heavy chain variable region comprising the sequence of SEQ ID NO: 99; (c) a light chain variable region comprising the sequence of SEQ ID NO: 48 and a heavy chain variable region comprising the sequence of SEQ ID NO: 100; (d) a light chain variable region comprising the sequence of SEQ ID NO: 49 and a heavy chain variable region comprising the sequence of SEQ ID NO: 101; or (e) a light chain variable region comprising the sequence of SEQ ID NO: 50 and a heavy chain variable region comprising the sequence of SEQ ID NO: 102. In other embodiments of the detection methods of the invention, the antibody in the detection reagent comprises: (a) a light chain comprising the sequence of SEQ ID NO: 111 and a heavy chain comprising the sequence of SEQ ID NO: 124; (b) a light chain comprising the sequence of SEQ ID NO: 112 and a heavy chain comprising the sequence of SEQ ID NO: 125; (c) a light chain comprising the sequence of SEQ ID NO: 113 and a heavy chain comprising the sequence of SEQ ID NO: 126; (d) a light chain comprising the sequence of SEQ ID NO: 114 and a heavy chain comprising the sequence of SEQ ID NO: 127; or (e) a light chain comprising the sequence of SEQ ID NO: 115 and a heavy chain comprising the sequence of SEQ ID NO: 128.

In certain embodiments, the chemically-modified nucleic acid molecule to be detected is conjugated to an antibody or antigen-binding fragment thereof. In such embodiments, the binding partner in the detection reagent can be a target antigen (or fragment of the antigen containing the epitope) of the antibody or antigen-binding fragment. In other such embodiments, the binding partner in the detection reagent is an anti-idiotypic antibody. An anti-idiotypic antibody is an antibody that binds to the idiotype of another antibody. An idiotype of an antibody is the specific combination of idiotopes present within the antibody's variable regions. In still other such embodiments, the binding partner in the detection reagent is a protein that specifically binds to the Fc region of the antibody, such as an anti-Fc region antibody, protein A, or protein G. In some such embodiments, the capture antibody immobilized to a surface is one of the pan-specific antibodies described herein (e.g. 14K10 antibody) and the antibody in the detection reagent is an anti-Fc region antibody. See, e.g., FIG. 5C. In certain embodiments of the detection methods of the invention, the chemically-modified nucleic acid molecule to be detected is conjugated to a protein and the binding partner in the detection reagent is an antibody or antigen-binding fragment thereof that specifically binds to the protein.

The detectable label in the detection reagent can be any of the detectable labels described above. In some embodiments, the detectable label in the detection reagent is a radiolabel, an enzyme, a fluorophore, a chromophore, a chemiluminescent label, an electrochemiluminescence (ECL) luminophore, a metallic nanoparticle, or a metallic nanoshell. In certain embodiments, the detectable label in the detection reagent is a fluorophore (e.g. fluorescein, rhodamine, Alexa dyes molecules, etc.), metallic nanoparticle (e.g. gold nanoparticles, silver nanoparticles, composite nanoparticles, etc.), enzyme (e.g. alkaline phosphatase, horseradish peroxidase, beta-galactosidase, etc.), radiolabel (¹²⁵I, ¹³¹I, ³H, ³⁵S, etc.), or ECL luminophore (e.g. ruthenium complexes, iridium complexes, etc.). In one particular embodiment, the detectable label in the detection reagent is an ECL luminophore, such as a ruthenium complex.

In some embodiments, the detection reagent may comprise a second molecule that links the detectable label to the binding partner that specifically binds to the chemically-modified nucleic acid molecule. For instance, in embodiments where the binding partner that specifically binds to the chemically-modified nucleic acid molecule is an antibody, that antibody need not directly be covalently linked to a detectable label. Rather, a secondary antibody that is specific to antibodies of the species from which the first antibody is derived may be covalently linked to the detectable label. By way of illustration, where the binding partner that specifically binds to the chemically-modified nucleic acid molecule is one of the rabbit monoclonal antibodies described herein, a secondary antibody specific to rabbit antibodies can be covalently linked to the detectable label.

Following contact of the surface comprising the captured chemically-modified nucleic acid molecules with the detection reagent, the methods of the invention comprise detecting or measuring a signal from the detectable label in the detection reagent. A signal from the detectable label indicates that the target chemically-modified nucleic acid molecule is present in the sample. In some embodiments in which the chemically-modified nucleic acid molecule is conjugated to another molecule (e.g. GalNAc moiety or protein), a signal from the detectable label may also indicate that the target chemically-modified nucleic acid conjugate is intact, i.e. that the nucleic acid molecule remains covalently linked to the conjugate partner (i.e. GalNAc moiety or protein). The signal to be detected will depend on the type of detection label employed. For instance, signals from metallic nanoparticle or nanoshell labels can be detected by measuring the amount of light scattering or light absorption. Signals from fluorophores or ECL luminophores can be detected or measured as light intensity at particular emission wavelengths. When the detectable label is an enzyme, the signal is produced by adding a substrate of the enzyme that produces a detectable signal, such as a chromogenic, fluorogenic, or chemiluminescent substrate. Instruments, such as spectrophotometers, fluorescent/luminescent plate readers, and other instruments capable of detecting spectral and electrochemical changes are commercially available and known to those of skill in the art. In certain embodiments, detecting a signal from the detectable label provides a qualitative assessment (i.e. chemically-modified nucleic acid molecule is present in the sample). In other embodiments, detecting a signal from the detectable label provides a quantitative measurement of the amount of the chemically-modified nucleic acid molecule in the sample. For example, in certain embodiments, measurements of, e.g., light scattering, light absorption, or fluorescence/luminescence emission allows for the amount of chemically-modified nucleic acid molecule in the sample to be determined quantitatively. Such quantitation can be achieved by measuring the signal from the detectable label in samples containing known amounts of chemically-modified nucleic acid molecules, constructing calibration curves from the data, and determining the amount of chemically-modified nucleic acid molecules in a test sample from the calibration curves.

The detection methods of the invention can be used to assess the pharmacokinetic properties (e.g. bioavailability), metabolism, and distribution of chemically-modified nucleic acid molecules. For instance, samples taken at different time points from subjects administered a chemically-modified nucleic acid molecule can be evaluated in the detection methods of the invention to determine the half-life of the molecule in certain fluids or tissues or the time period when the molecule is distributed to certain tissue compartments. Thus, in certain embodiments, the present invention provides methods for assessing a pharmacokinetic profile of a chemically-modified nucleic acid molecule in a subject. In one embodiment, the method comprises administering the chemically-modified nucleic acid molecule to the subject; obtaining at least one sample from the subject following administration of the chemically-modified nucleic acid molecule; contacting the sample with at least one monoclonal antibody or antigen-binding fragment thereof of the invention (e.g. a pan-specific antibody) under conditions allowing the chemically-modified nucleic acid molecule, if present in the sample, to bind to the monoclonal antibody or antigen-binding fragment thereof, thereby forming a complex; and detecting the complex. Any of the assay formats described herein can be used to detect the complex of the monoclonal antibody or antigen-binding fragment thereof of the invention (e.g. a pan-specific antibody) and the chemically-modified nucleic acid molecule. Immunohistochemical methods using labeled forms of the monoclonal antibodies or antigen-binding fragments thereof of the invention as described herein can also be used to detect the complex of the monoclonal antibody or antigen-binding fragment thereof and the chemically-modified nucleic acid molecule. In some embodiments, a plurality of samples is obtained from the subject at different time points following administration of the chemically-modified nucleic acid molecules. In such embodiments, the level or concentration of the chemically-modified nucleic acid molecule in the samples can be measured to determine the change in concentration or level of the chemically-modified nucleic acid molecule over time. The samples can be bodily fluids, such as serum, plasma, urine, or blood. In other embodiments, the samples are tissue samples or tissue homogenates, such as tissue samples or homogenates from the liver, kidney, pancreas, or other organ. The subject can be a mammal, including a mouse, rat, dog, pig, or non-human primate (e.g. cynomolgus monkey). In some embodiments, the subject is a human.

The monoclonal antibodies or antigen-binding fragments of the invention can also be used in methods to detect anti-drug antibodies in subjects administered a chemically-modified nucleic acid-based drug. For example, labeled forms of the monoclonal antibodies or antigen-binding fragments thereof of the invention (e.g. pan-specific antibodies) can be used in a competitive immunoassay to detect the presence of anti-drug antibodies in a sample from a subject who has been administered a chemically-modified nucleic acid molecule. In such a competitive assay, the chemically-modified nucleic acid drug molecule is immobilized to a surface (e.g. a well of microtiter plate) and contacted with a sample obtained from a subject administered the chemically-modified nucleic acid molecule. A labeled form of a monoclonal antibody of the invention (e.g. a pan-specific monoclonal antibody coupled to a detectable label) is then added to the surface. If little or no anti-drug antibodies are present in the sample from the subject, the labeled monoclonal antibody will then be able to bind to the available chemically-modified nucleic acid molecule immobilized to the surface. If anti-drug antibodies are present in the sample, they will bind to the immobilized chemically-modified nucleic acid molecules leaving few or no available immobilized chemically-modified nucleic acid molecules to bind to the labeled monoclonal antibodies. Thus, any anti-drug antibodies present in the sample will compete with the labeled monoclonal antibodies for binding to the immobilized chemically-modified nucleic acid molecule. The signal from the detectable label on the monoclonal antibody of the invention will inversely correlate with the amount of anti-drug antibody in the subject's sample (i.e. a decrease in signal from the detectable label relative to a control with no sample indicates that the test sample contains anti-drug antibodies). Methods of immobilizing nucleic acid molecules to a surface are known to those of skill in the art and can include any of the methods described above for attaching nucleic acid molecules to nanobeads.

Accordingly, the present invention includes a method for detecting an anti-drug antibody to a chemically-modified nucleic acid molecule in a subject. In some embodiments, the method comprises providing a surface comprising the chemically-modified nucleic acid molecule; contacting the surface with a sample obtained from a subject administered the chemically-modified nucleic acid molecule; contacting the surface with a detection reagent, wherein the detection reagent comprises a monoclonal antibody of the invention coupled to a detectable label; and detecting a signal from the detectable label, wherein a signal from the detectable label is indicative of the absence of anti-drug antibody in the sample. In certain embodiments, the detection reagent comprises any one of the pan-specific antibodies of the invention coupled to a detectable label. In one particular embodiment, the detection reagent comprises the 14K10 antibody coupled to a detectable label. In other embodiments, the chemically-modified nucleic acid molecule is the 1851 RNAi construct and the detection reagent comprises any one of the 1851 RNAi construct-specific antibodies of the invention coupled to a detectable label. In some embodiments of the anti-drug antibody detection methods of the invention, the sample is a serum or plasma sample.

The methods of the invention can be used to detect or measure chemically-modified nucleic acid molecules in various types of samples. In some embodiments, the sample is a bodily fluid, such as blood, serum, plasma, cerebral spinal fluid, saliva, or urine. In other embodiments, the sample is a tissue (e.g. tissue homogenate), a cell lysate, or a sub-cellular fraction. The tissue may be from any organ, including, but not limited to, liver, kidney, pancreas, lung, or skin. In certain embodiments, the sample (bodily fluid, tissue, or cell sample) is obtained from an animal or human subject who has been administered the chemically-modified nucleic acid molecule. The sample may be obtained from a subject prior to, during, or after treatment with the chemically-modified nucleic acid molecule. In some embodiments, the samples are obtained from cell cultures that have been exposed to the chemically-modified nucleic acid molecules. In such embodiments, the sample may be a supernatant of the cell culture, a lysate of the cells in the culture, or a sub-cellular fraction of the cells in the culture.

Any type of chemically-modified nucleic acid molecule, such as those described herein, can be detected or measured in a sample according to the methods of the invention. In some embodiments, the chemically-modified nucleic acid molecule comprises one or more modified nucleotides selected from 2′-fluoro modified nucleotides, 2′-O-methyl modified nucleotides, 2′-O-methoxyethyl modified nucleotides, 2′-O-alkyl modified nucleotides, 2′-O-allyl modified nucleotides, BNAs, or combinations thereof. The chemically-modified nucleic acid molecules may also comprise one or more phosphorothioate internucleotide linkages.

In certain embodiments, the chemically-modified nucleic acid molecule to be detected or measured according to the methods of the invention is double-stranded. In some such embodiments, the chemically-modified nucleic acid molecule is an RNAi construct comprising a sense strand and an antisense strand. The sense strand and an antisense strand of the RNAi construct can each independently be about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length. In some embodiments, the RNAi construct is therapeutic (i.e. targeted to a gene or RNA molecule associated with a disease or disorder). The RNAi constructs can be any of the RNAi constructs listed in Table 6. In one embodiment, the RNAi construct is the 1851 RNAi construct (sense strand comprising the sequence of SEQ ID NO: 181 and antisense strand comprising the sequence of SEQ ID NO: 192).

In some embodiments, the chemically-modified nucleic acid molecules to be detected or measured according to the methods of the invention are covalently linked to a ligand, such as any of the ligands described above. In some such embodiments, the ligand comprises a GalNAc moiety. The GalNAc moiety can be a multivalent GalNAc moiety, such as a trivalent or tetravalent GalNAc moiety. In one embodiment, the GalNAc moiety has the structure of Structure 1. In another embodiment, the GalNAc moiety is the TL01 GalNAc moiety. In yet another embodiment, the GalNAc moiety is the TL02 GalNAc moiety. In still another embodiment, the GalNAc moiety is the TL03 GalNAc moiety.

The present invention includes kits for detecting chemically-modified nucleic acid molecules in a sample. In one embodiment, the kit comprises a capture antibody immobilized to a surface, wherein the capture antibody specifically binds to the chemically-modified nucleic acid molecule; a detection reagent comprising a detectable label coupled to a binding partner that specifically binds to the chemically-modified nucleic acid molecule; and instructions for contacting the sample with the immobilized capture antibody and detection reagent, and instructions for detecting a signal from the detectable label. Any of the capture antibodies and binding partners described above for use in the methods of the invention can be the capture antibodies and binding partners included in the kits.

In certain embodiments of the kits of the invention, the capture antibody is one of the pan-specific antibodies described herein, for example, the 14K10, 14F4, or 5117 antibody. In one embodiment, the capture antibody comprises a CDRL1 of SEQ ID NO: 1, a CDRL2 of SEQ ID NO: 14, a CDRL3 of SEQ ID NO: 25, a CDRH1 of SEQ ID NO: 51, a CDRH2 of SEQ ID NO: 64, and a CDRH3 of SEQ ID NO: 77. In a related embodiment, the capture antibody comprises a light chain variable region comprising the sequence of SEQ ID NO: 38 and a heavy chain variable region comprising the sequence of SEQ ID NO: 90. In another related embodiment, the capture antibody comprises a light chain comprising the sequence of SEQ ID NO: 103 and a heavy chain comprising the sequence of SEQ ID NO: 116.

In some embodiments, the detection reagent in the kits comprises one of the pan-specific antibodies described herein (e.g. 14K10, 14F4, or 5117 antibody) coupled to a detectable label. In one such embodiment, the antibody coupled to a detectable label comprises a CDRL1 of SEQ ID NO: 1, a CDRL2 of SEQ ID NO: 14, a CDRL3 of SEQ ID NO: 25, a CDRH1 of SEQ ID NO: 51, a CDRH2 of SEQ ID NO: 64, and a CDRH3 of SEQ ID NO: 77. In another such embodiment, the antibody coupled to a detectable label comprises a light chain variable region comprising the sequence of SEQ ID NO: 38 and a heavy chain variable region comprising the sequence of SEQ ID NO: 90. In yet another such embodiment, the antibody coupled to a detectable label comprises a light chain comprising the sequence of SEQ ID NO: 103 and a heavy chain comprising the sequence of SEQ ID NO: 116.

In other embodiments, the detection reagent in the kits comprises one of the GalNAc moiety-specific antibodies described herein (e.g. the 14D4, 1613, 16A22, 17D13, or 18J5 antibody) coupled to a detectable label. In one such embodiment, the antibody coupled to a detectable label comprises a CDRL1 of SEQ ID NO: 9, a CDRL2 of SEQ ID NO: 21, a CDRL3 of SEQ ID NO: 33, a CDRH1 of SEQ ID NO: 59, a CDRH2 of SEQ ID NO: 72, and a CDRH3 of SEQ ID NO: 85. In another such embodiment, the antibody coupled to a detectable label comprises a light chain variable region comprising the sequence of SEQ ID NO: 46 and a heavy chain variable region comprising the sequence of SEQ ID NO: 98. In yet another such embodiment, the antibody coupled to a detectable label comprises a light chain comprising the sequence of SEQ ID NO: 111 and a heavy chain comprising the sequence of SEQ ID NO: 124.

The detectable label coupled to the antibodies can be any of the detectable labels described herein. In some embodiments, the detectable label is a fluorophore, metallic nanoparticle, enzyme, radiolabel, or ECL luminophore. In one particular embodiment, the detectable label in the detection reagent is an ECL luminophore, such as a ruthenium complex.

In certain embodiments of the kits of the invention, the surface comprising the capture antibody can be a bead or particle, a membrane, a tube, a resin, a column, an electrode, or a well in an assay plate (e.g. a well in a microtiter plate). In one particular embodiment, the surface is a well in a microtiter plate.

The following examples, including the experiments conducted and the results achieved, are provided for illustrative purposes only and are not to be construed as limiting the scope of the appended claims.

### EXAMPLES

### Example 1. Generation of Monoclonal Antibodies to Chemically-Modified RNAi Constructs

To generate monoclonal antibodies to a trivalent N-acetyl-galactosamine (GalNAc)-conjugated RNAi construct, two different immunogens were designed and synthesized. The first immunogen comprised the GalNAc-conjugated RNAi construct 1851 conjugated to keyhole limpet hemocyanin (KLH) protein (KLH-1851 immunogen). The second immunogen comprised streptavidin nanobeads coated with biotinylated RNAi construct 1851 (Bead-1851 immunogen). Specifically, the 1851 RNAi construct was biotinylated at the 5′ end of the antisense strand via a PEG4-carbon six linker and mixed with streptavidin nanobeads having a mean diameter of 0.1 μm (Bangs Laboratories, Inc.; Catolog No. CP01000) in a 1:4 (w/w) ratio (RNAi constructs:nanobeads) (FIG. 1). The second immunogen was designed to present the oligonucleotide antigen in a multivalent form with a fixed orientation and spacing. The nanobeads used to create the second immunogen contained approximately 1600 to 2000 streptavidin groups per bead, which resulted in an equivalent density and rigidity of antigen display that was not achievable using conjugation to a carrier protein like KLH. The size and shape of the nanobeads were also believed to reduce the in vivo clearance rate of the immunogen to increase the exposure of the immunogen to the immune system.

The nucleotide sequences for the sense and antisense strands of RNAi construct 1851 as well as the structure of the GalNAc moiety are provided in Table 6 below. RNAi constructs were synthesized using solid phase phosphoramidite chemistry. Synthesis was performed on a MerMade12 (Bioautomation) instrument. A trivalent GalNAc moiety was conjugated to the 5′ end of the sense strand following automated synthesis. Following purification by anion exchange chromatography and desalting by size exclusion chromatography, the sense strand and antisense strand were annealed to create the duplex by mixing the strands in an equimolar ratio, heating the mixture to 90° C. for 5 min, and allowing the mixture to cool to room temperature.

Rabbits were immunized subcutaneously with either of the two immunogens on day 0, 7, 21, 42, 63, 91, 119, and 147. For the first injection, 100-200 μg of the immunogens was administered to the animals in combination with Complete Freund's Adjuvant. For all subsequent injections, 25-100 μg of the immunogens was administered in combination with Incomplete Freund's Adjuvant. Blood was collected from the animals at 7 and 14 days after each injection starting at day 28 to determine antibody titers and antigen specificity by ELISA. At the end of the immunization protocol, spleens were harvested from the animals, dissociated, and frozen. Thawed rabbit cells were single cell sorted on a FACS Aria III instrument into 384-well plates on biotinylated RNAi construct 1851 (detected by streptavidin conjugated to Alexa Fluor 647) and on anti-rabbit IgG antibody conjugated to Alexa Fluor 488 to select IgG positive, antigen-specific cells. Each well of the microtiter plate into which the cells were sorted contained RPMI media supplemented with fetal bovine serum (FBS), 10% activated rabbit splenocyte supernatant, and feeder cell culture. After 7 days in monoclonal culture and B-cell expansion, culture supernatants were collected for subsequent assays as described in more detail below.

Culture supernatants from rabbit B-cell expansion were screened for specificity in a standard colorimetric ELISA format. Four biotin-conjugated RNAi constructs were used for the screening assays: (i) 1851 RNAi construct (with GalNAc moiety), (ii) 1851 RNAi construct without GalNAc moiety, (iii) 6189 RNAi construct (with GalNAc moiety), and (iv) 6189 RNAi construct without GalNAc moiety. The 6189 RNAi construct has the same GalNAc moiety, chemical modification pattern, and format as the 1851 RNAi construct, but has different nucleotide sequences in the sense and antisense strands. The structure of the 6189 RNAi construct is provided in Table 6. For the RNAi constructs containing a trivalent GalNAc moiety at the 5′ end of the sense strand, biotin was covalently attached to the 5′ end of the antisense strand via a PEG4-carbon six linker. For the RNAi constructs lacking a GalNAc moiety, biotin was covalently attached to the 5′ end of the sense strand via a PEG4-carbon six linker. The biotinylated molecules were captured on neutravidin-coated microtiter plates and blocked with 1% non-fat dry milk in phosphate buffered saline. Culture supernatants were diluted 1:5 and added to each well. Antibody binding was determined using a secondary anti-rabbit IgG Fc antibody conjugated to horse radish peroxidase (HRP) and a TMB substrate (3,3′,5,5′-tetramethylbenzidine) followed by quenching with hydrochloric acid. The supernatants were screened initially with the 1851 RNAi construct (with GalNAc moiety) to confirm antigen-specific binding. Following this primary screen, the supernatants were then screened with all four antigens described above in a secondary screening assay to more specifically ascertain the antibody binding characteristics.

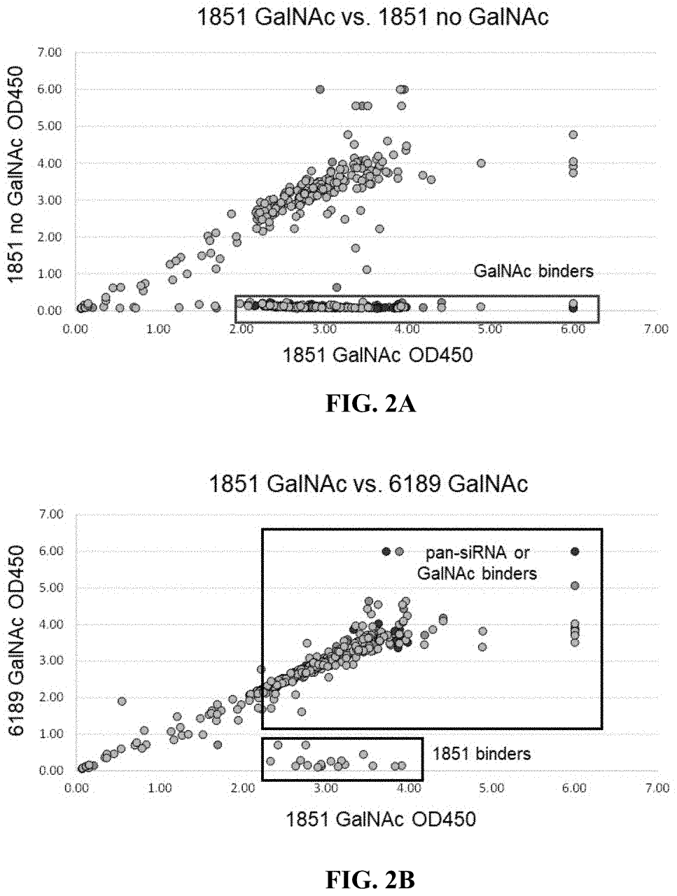

The results of the ELISA secondary screening assay revealed that the monoclonal antibodies fell into three separate categories in terms of binding specificity. Antibodies in the first category appeared to specifically recognize the GalNAc moiety (FIG. 2A). Antibodies in the second category specifically recognized the 1851 RNAi construct, whereas antibodies in a third category appeared to recognize the double-stranded RNA portion of the construct regardless of sequence (FIG. 2B). A summary of results of the ELISA screening assays illustrating the binding specificity for the rabbit monoclonal antibodies obtained from three different animals is provided in Table 5 below.

TABLE 5
Monoclonal Antibody Binding Specificity by ELISA

| | | | Secondary Screen | | |
|---|---|---|---|---|---|
| | | Antigen- | | 1851 | |
| | | specific | GalNAc | RNAi | |
| | | hits in | moiety- | construct- | Binders |
| Animal | | primary | specific | specific | to all four |
| No. | Immunogen | screen | binders | binders | antigens |
| 1 | KLH-1851 | 59 | 50 | 1 | 5 |
| 2 | KLH-1851 | 52 | 50 | 0 | 0 |
| 3 | Bead-1851 | 294 | 74 | 16 | 192 |

As shown in Table 5, the bead-based form of the RNAi construct was much more effective at generating antigen-specific antibodies than the RNAi construct conjugated to the KLH carrier protein, as the bead-based immunogen produced nearly a 5-fold greater number of antigen-specific antibodies. Moreover, antibodies generated from the Bead-1851 immunogen had a range of binding specificities, whereas antibodies generated with the KLH-1851 immunogen primarily recognized the GalNAc moiety of the RNAi construct. All but one of the 1851 RNAi construct-specific antibodies were generated using the bead-based form of the antigen. In addition, robust antibody titers were observed from rabbits immunized with the Bead-1851 immunogen as early as 42 days after initial immunization. These antibody titers were 70-fold to 250-fold greater than the titers observed for rabbits immunized with the KLH-1851 immunogen at the same time point (day 42; data not shown). Thus, this immunization method using multivalent nucleic acid-displaying nanobeads as an immunogen has proven to be a particularly effective way to produce monoclonal antibodies that bind to different aspects of nucleic acid molecules, which are known to be poorly immunogenic.

Rabbit B-cells producing the top antibody binders in each of the three binding specificity categories (e.g. GalNAc moiety-specific, 1851 RNAi construct-specific, and pan-RNAi construct-specific) were lysed for antibody sequencing and cloning. The monoclonal antibodies were recombinantly expressed in HEK 293-6E cells and purified. The amino acid sequences for select antibodies in each of the binding specificity categories are shown in Tables 1A-1B (CDR and variable regions) and Table 2 (full heavy and light chains). Nucleotide sequences encoding the variable regions and full chains for the antibodies are provided in Tables 3 and 4, respectively. The purified antibodies were evaluated to confirm binding specificity in further validation screens. In the validation screens, each of the antibodies was tested at three different concentrations (5, 1, and 0.2 μg/mL) for binding to each of the following antigens, which were attached by biotinylation to streptavidin-coated beads:
(i) 1851 RNAi construct (with GalNAc moiety);
(ii) 1851 RNAi construct without GalNAc moiety;
(iii) 6189 RNAi construct (with GalNAc moiety);
(iv) 6189 RNAi construct without GalNAc moiety; and
(v) β-GalNAc3 dendrimer

The β-GalNAc3 dendrimer comprised the following structure:

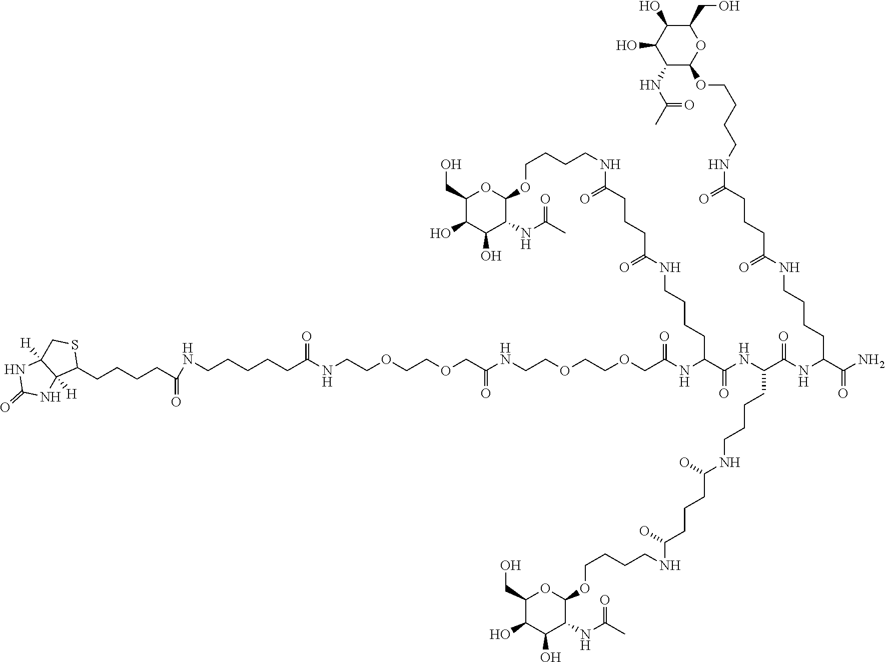

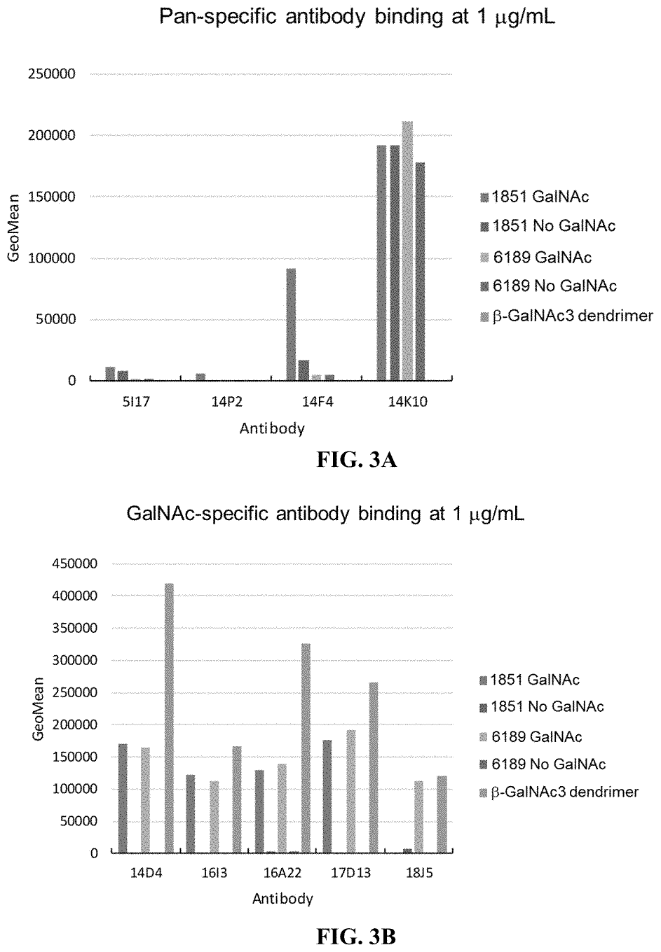

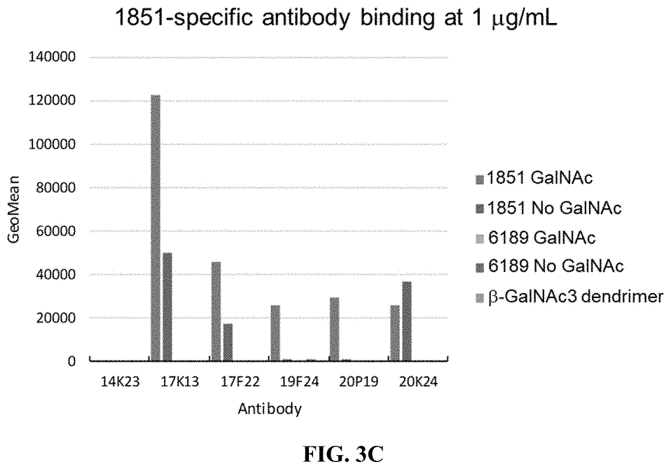

Antibody binding was detected by an anti-rabbit IgG antibody conjugated to Alexa Fluor 488 and analyzed on an Intellicyt iQue Screener flow cytometer. For the pan-RNAi construct-specific antibodies that appeared to recognize the double-stranded RNA structure of the molecule irrespective of nucleotide sequence, at a concentration of 1 μg/mL, one out of the four recombinant monoclonal antibodies tested (14K10 antibody) bound nearly equivalently to the four antigens comprising the double-stranded RNA structure and did not bind to the β-GalNAc3 dendrimer antigen, which lacked the RNA component (FIG. 3A). All five GalNAc moiety-specific recombinant monoclonal antibodies specifically recognized all antigens comprising the GalNAc moiety and did not bind to the two antigens lacking the GalNAc moiety (FIG. 3B). Note the lack of binding of the 18J5 antibody to the 1851 GalNAc antigen in FIG. 3B was the result of a technical error as binding of this antibody was observed at the other two antibody concentrations (data not shown). For the antibodies that appeared to be specific for the 1851 RNAi construct in initial screening, five of the six recombinant monoclonal antibodies that were evaluated were confirmed to bind specifically to the 1851 RNAi construct and not to the 6189 RNAi construct, which had different nucleotide sequences in each of the strands (FIG. 3C). The 19F24 and 20P19 antibodies only recognized the 1851 RNAi construct when the GalNAc moiety was present, suggesting that the epitope for these two antibodies may include some aspect of the GalNAc moiety structure in combination with part of the nucleobase structure of the RNAi construct. Interestingly, neither 19F24 nor 20P19 bound to the 6189 RNAi construct with the GalNAc moiety or the β-GalNAc3 dendrimer, ruling out that these two antibodies bind entirely to the GalNAc moiety.

To further evaluate the binding specificity of the pan-specific and 1851 RNAi construct-specific antibodies, each of the recombinant antibodies was evaluated in a competition binding assay. Three different RNAi constructs (1851, 1907, and 1418), the sequences of which are provided in Table 6, were preincubated with each antibody at a 55:1 molar ratio (RNAi construct:antibody) followed by exposure to streptavidin beads coated with biotinylated 1851 RNAi construct. The 1851 and 1907 RNAi constructs had the same core nucleotide sequences but different end structures with the 1851 construct having two blunt ends and the 1907 construct having two-nucleotide overhangs at the 3′ end of each strand. The 1418 construct had different nucleotide sequences than the 1851 and 1907 constructs as well as a different GalNAc moiety. Antibody binding to the 1851 RNAi construct-coated beads was detected by an anti-rabbit IgG antibody conjugated to Alexa Fluor 488 and analyzed on an Intellicyt iQue Screener flow cytometer. If the antibodies bound to the RNAi constructs during preincubation and thus competed with the 1851 RNAi construct-coated beads for binding to the antibodies, a decrease in binding signal to the beads was observed as compared to the binding signal in the absence of the RNAi constructs in the preincubation, which is represented as a percent inhibition in the figures. A higher percent inhibition reflects antibody binding to the competing RNAi construct.

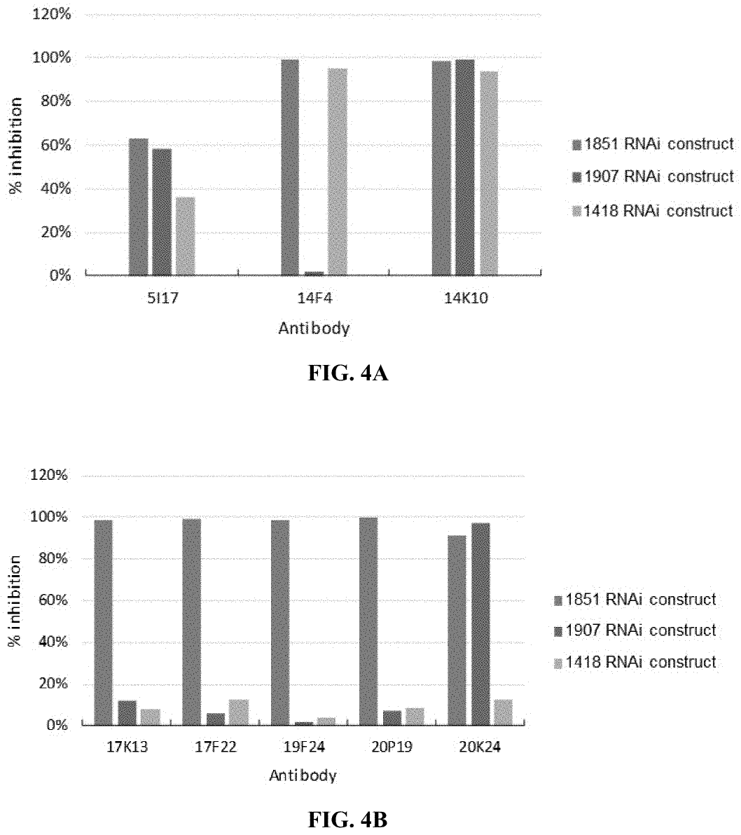

The results of the competition assay for the pan-specific antibodies and the 1851 RNAi construct-specific antibodies are shown in FIGS. 4A and 4B, respectively. Of the three recombinant pan-specific antibodies tested, all three RNAi constructs effectively inhibited binding of the 14K10 antibody to the 1851 RNAi construct-coated bead, confirming that this antibody recognizes some feature of the double-stranded RNA structure independent of nucleotide sequence (FIG. 4A). The 5117 antibody was also inhibited by all three RNAi constructs, but to a lesser degree than the 14K10 antibody. The 14F4 antibody exhibited significant binding to the 1851 and 1418 RNAi constructs, which had distinct nucleotide sequences and GalNAc moieties. However, 14F4 exhibited weak binding to the 1907 RNAi construct, which had a similar core nucleotide sequence as the 1851 RNAi construct.

As shown in FIG. 4B, all five 1851 RNAi construct-specific antibodies were inhibited from binding the antigen-coated beads by the 1851 RNAi construct, but not the 1418 RNAi construct, which had different nucleotide sequences. Four out of the five antibodies were also not inhibited from binding the antigen-coated beads by the 1907 RNAi construct, suggesting that these four antibodies are specific to the 1851 RNAi construct. The binding of the 20K24 antibody to the antigen-coated beads was inhibited by the 1907 RNAi construct as well as the 1851 RNAi construct. Because these two constructs share a common core sequence, this result suggests that the 20K24 antibody recognizes a particular contiguous sequence of nucleotides that is common to both constructs.

The results of the experiments described in this Example show that the described immunization method using multivalent nucleic acid-displaying nanobeads as an immunogen can effectively generate monoclonal antibodies that bind to unique structural features of the chemically-modified RNAi constructs. Antibodies with three different binding specificities were produced: (i) antibodies that bound to the double-stranded RNA structure of the RNAi constructs irrespective of nucleotide sequence (i.e. “pan-specific” antibodies), (ii) antibodies that specifically recognized the 1851 RNAi construct, and (iii) antibodies that specifically bound to the GalNAc moiety of the RNAi constructs. These antibodies can be used in a variety of assays to detect chemically-modified RNAi constructs or their metabolites in biological samples as described in more detail in Example 2.

### RNAi Constructs

Table 6 below lists the modifications in the sense and antisense sequences and the structure and site of conjugation for the GalNAc moiety for each of the GalNAc-conjugated RNAi constructs employed in the experiments described in the Examples. The nucleotide sequences in Table 6 are listed according to the following notations: A, U, G, and C=corresponding ribonucleotide; dT, dA, dG, dC=corresponding deoxyribonucleotide; a, u, g, and c=corresponding 2′-O-methyl ribonucleotide; Af, Uf, Gf, and Cf=corresponding 2′-deoxy-2′-fluoro (“2′-fluoro”) ribonucleotide; Phos=terminal nucleotide has a monophosphate group at its 5′ end; invAb=inverted abasic nucleotide (i.e. abasic nucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand); and invdX=inverted deoxyribonucleotide (i.e. deoxyribonucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand). Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. GalNAc structures are shown below Table 6. The TL01 and TL03 GalNAc moieties were conjugated to the sense strands of the RNAi constructs via a phosphorothioate linkage, whereas the TL02 GalNAc moiety was conjugated to the sense strand via a phosphodiester linkage.

TABLE 6
Exemplary Chemically-Modified RNAi Constructs

| | | Site of | | | | |
|---|---|---|---|---|---|---|
| Con- | | GalNAc | | SEQ | | SEQ |
| struct | GalNAc | Moiety | | ID | | ID |
| No. | Moiety | Conjug. | Sense Sequence (5′-3′) | NO: | Antisense Sequence (5′-3′) | NO: |
| 1851 | TL01 | 5′ end | csagccccuUfAfUfuguuauacgs | 181 | usCfsgUfaUfaacaaUfaAfgGfgGfcsUfsg | 192 |
| | | of sense | [invdA] | | | |
| | | strand | | | | |
| 6189 | TL01 | 5′ end | usucuuucuCfAfAfgaacgcugcs | 182 | usGfscAfgCfguucuUfgAfgAfaAfgsAfsa | 193 |
| | | of sense | [invdA] | | | |
| | | strand | | | | |
| 1907 | TL01 | 5′ end | gsccccuUfAfUfuguuauacgauus | 183 | usCfsgUfaUfaacaaUfaAfgGfgGfcsusu | 194 |
| | | of sense | [invAb] | | | |
| | | strand | | | | |
| 1418 | TL02 | 3′ end | [phos]GfsusGfgGfaAfgAfAf | 184 | [phos]asCfsuUfcAfuCfuuuCfuUfc | 195 |
| | | of sense | AfgAfuGfaAfgUfuUf | | CfcAfcsUfsu | |
| | | strand | | | | |
| 16081 | TL03 | 5′ end | gaggacugUfgCfCfCfAfcuucacs | 185 | asGfsugaaGfugggCfaCfaguccucsusu | 196 |
| | | of sense | [invAb] | | | |
| | | strand | | | | |
| 16082 | TL03 | 5′ end | augugggaAfgAfAfAfGfaugaags | 186 | asCfsuucaUfcuuuCfuUfcccacaususu | 197 |
| | | of sense | [invAb] | | | |
| | | strand | | | | |
| 16083 | TL03 | 5′ end | cccuaucaUfgAfCfCfAfaggagus | 187 | usAfscuccUfugguCfaUfgauagggsusu | 198 |
| | | of sense | [invAb] | | | |
| | | strand | | | | |
| 16084 | TL03 | 5′ end | gggaagaaAfgAfUfGfAfagucgcs | 188 | asGfscgacUfucauCfuUfucuucccsusu | 199 |
| | | of sense | [invAb] | | | |
| | | strand | | | | |
| 7213 | TL03 | 5′ end | [invAb]augccuUfuCfUfAfCfag | 189 | asGfscCfaCfUfguagAfaAfggcaususu | 200 |
| | | of sense | UfgGfcUfsusUf | | | |
| | | strand | | | | |
| 8172 | TL03 | 5′ end | uccuaugaCfuGfUfAfGfauuuuas | 190 | asUfsaaaaUfcuacAfgUfcauaggasusu | 201 |
| | | of sense | [invAb] | | | |
| | | strand | | | | |
| 10927 | TL03 | 5′ end | acacaaUfgCfUfCfAfgacgcaaus | 191 | usUfsgcguCfugagCfaUfugugususu | 202 |
| | | of sense | [invAb] | | | |
| | | strand | | | | |

The structure of the TL01 GalNAc moiety is:

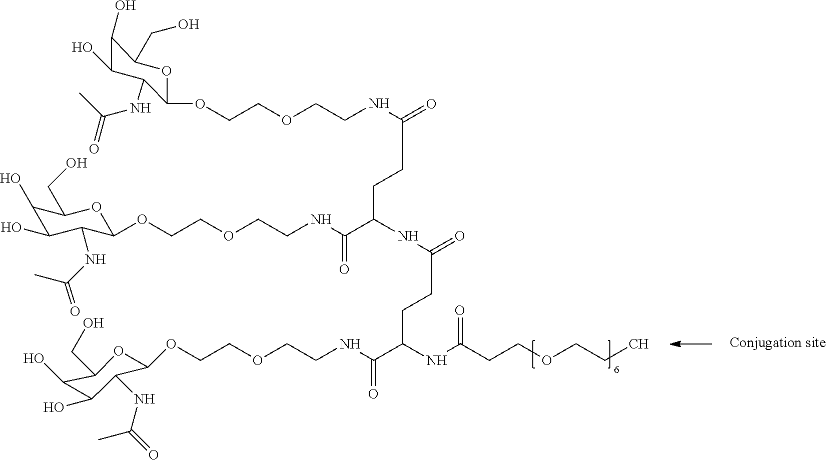

where the arrow denotes the site of conjugation to the nucleic acid molecule via a phosphorothiodiester bond.

The structure of the TL02 GalNAc moiety is:

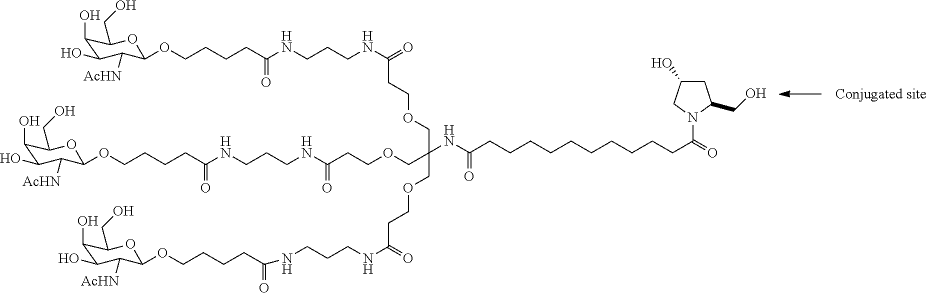 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O]

where the arrow denotes the site of conjugation to the nucleic acid molecule via a phosphodiester bond. “Ac” represents an acetyl group.

The structure of the TL03 GalNAc moiety is:

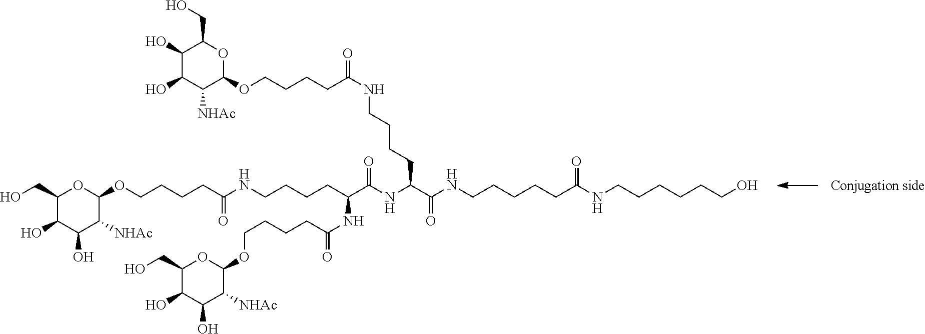 [SMILES: CC(=O)N[C@H]1[C@H](OCCCCC(=O)NCCCC[C@H](NC(=O)CCCCO[C@@H]2O[C@H](CO)[C@H](O)[C@H](O)[C@H]2NC(C)=O)C(=O)N[C@@H](CCCCNC(=O)CCCCO[C@@H]2O[C@H](CO)[C@H](O)[C@H](O)[C@H]2NC(C)=O)C(=O)NCCCCCC(=O)NCCCCCCO)O[C@H](CO)[C@H](O)[C@@H]1O]

where the arrow denotes the site of conjugation to the nucleic acid molecule via a phosphorothiodiester bond. “Ac” represents an acetyl group.

### Example 2. Immunoassays for Detection of Chemically-Modified RNAi Constructs

Three different immunoassays using one or more of the antibodies described in Example 1 were developed to detect chemically-modified RNAi constructs in biological samples, such as serum or tissue homogenates.

### Total Drug Assay

The first of such assays, referred to as a total drug assay, uses a pan-RNAi construct-specific antibody described in Example 1, such as the 14K10 antibody, as both a capture reagent and detection reagent to detect and quantify chemically-modified RNAi constructs regardless of nucleotide sequence in various samples. One embodiment of the total drug assay is shown schematically in FIG. 5A. In this embodiment, the 14K10 pan-RNAi construct-specific antibody was biotinylated and added at a concentration of 1 μg/mL in blocking buffer (5% nonfat powdered milk in Tris buffered saline (Blocker™ BLOTTO, ThermoFisher Scientific) to the wells of a streptavidin-coated gold microtiter plate (Meso Scale Diagnostics (MSD) GOLD™ streptavidin plate) and the plate was shaken for 30 minutes at room temperature. The plate was then washed with wash buffer (imidazole-buffered saline and Tween 20; supplied as a 20× Wash Solution Concentrate from KPL Inc.). Biological samples (serum, plasma, or tissue homogenate) were diluted 1:20 in blocking buffer and added to the wells of the microtiter plate and incubated for 1 hour at room temperature. The plate was again washed with wash buffer. Subsequently, a ruthenium-labeled 14K10 pan-RNAi construct-specific antibody was added at a concentration of 0.5 μg/mL to each of the samples in blocking buffer and incubated for 1 hour at room temperature. After washing the plate with wash buffer, the signal from the ruthenium label was read using an MSD electro-chemiluminescent reader (MSD Sector 5 600) and MSD Read Buffer T with surfactant.

### Intact Drug Assay for GalNAc-conjugated RNAi Constructs

Another immunoassay incorporating antibodies of the invention can be used to detect and quantify intact GalNAc-conjugated RNAi constructs (i.e. GalNAc moiety is still conjugated to RNA strand) in various biological samples. In this assay format, a pan-RNAi construct-specific antibody, such as the 14K10 antibody, is used as the capture reagent and a GalNAc moiety-specific antibody, such as the 14D4 antibody, is used as the detection reagent. This assay format is particularly useful for pharmacokinetic and metabolism studies to track when and where the GalNAc moiety is removed from the chemically-modified RNAi construct. One embodiment of this intact GalNAc-RNAi construct assay is illustrated schematically in FIG. 5B.

The assay procedure is similar to that described above for the total drug assay except that a GalNAc moiety-specific antibody of the invention is used as the detection antibody. Specifically, biotinylated 14K10 pan-specific antibody was added at a concentration of 1 μg/mL in blocking buffer to the wells of a streptavidin-coated gold microtiter plate (MSD GOLD™ streptavidin plate) and the plate was shaken for 30 minutes at room temperature. The plate was then washed with wash buffer. Biological samples (serum, plasma, or tissue homogenate) were diluted 1:20 in blocking buffer and added to the wells of the microtiter plate and incubated for 1 hour at room temperature. The plate was again washed with wash buffer. The 14D4 antibody, which specifically binds to the GalNAc moieties of the chemically-modified RNAi constructs, was labeled with ruthenium and added at a concentration of 0.5 μg/mL to each of the samples in blocking buffer and incubated for 1 hour at room temperature. After washing the plate with wash buffer, the signal from the ruthenium label was read using an MSD electro-chemiluminescent reader (MSD Sector 5 600) and MSD Read Buffer T with surfactant.

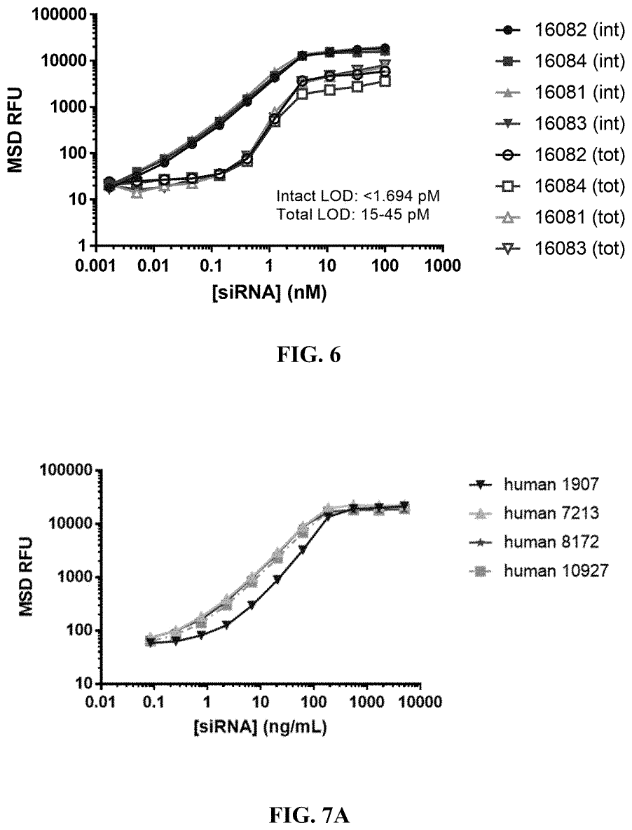

Both of the assay formats described above were evaluated with four different GalNAc-conjugated RNAi constructs (construct nos. 16081, 16082, 16083, and 16084) to determine the ability of the assays to detect both total drug and GalNAc-intact drug in human serum. The sequences for the GalNAc-conjugated RNAi constructs are provided in Table 6 above. Each of the GalNAc-conjugated RNAi constructs was diluted in human serum to generate an eleven-point standard curve (100 nM to 1.7 pM). The results of the two assays are shown in FIG. 6. The limit of detection (LOD) for the intact drug assay for all four constructs was less than 1.7 pM, whereas the LOD for the total drug assay was from about 15 to 45 pM for the different constructs. The results show that both assay formats can detect picomolar concentrations of the RNAi constructs in a biological matrix independent of the nucleotide sequences of the RNAi constructs.

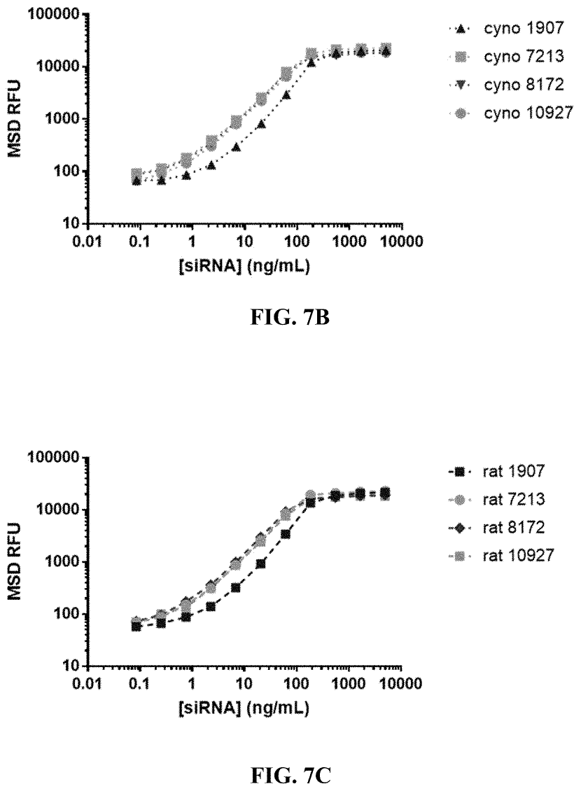

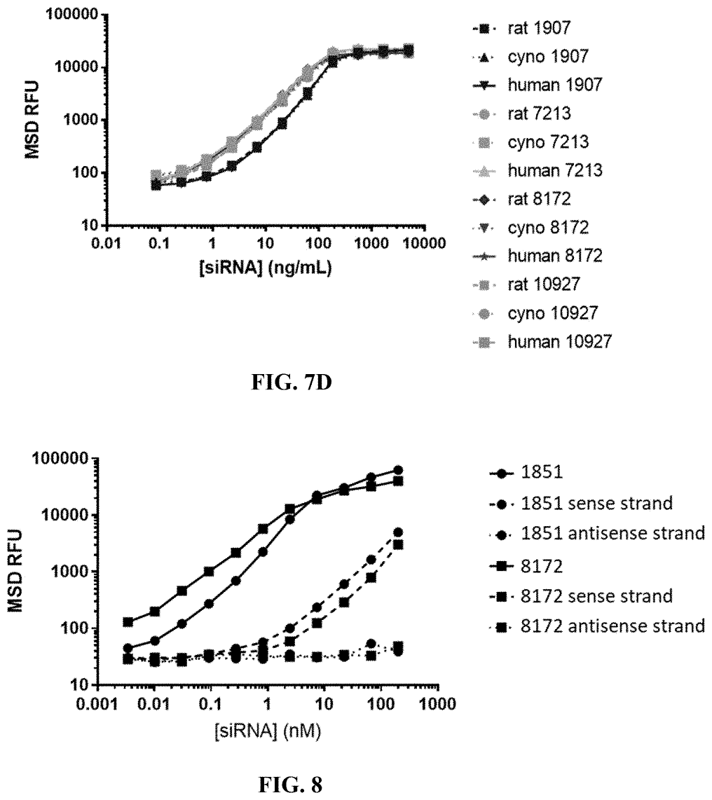

The GalNAc intact assay was further evaluated for the ability to detect additional GalNAc-conjugated RNAi constructs with distinct nucleotide sequences in matrices from three different species. Specifically, GalNAc-conjugated RNAi constructs 1907, 7213, 8172, and 10927, the sequences for which are provided in Table 6 above, were diluted in serum from humans, cynomolgus monkeys, or rats to generate an eleven-point standard curve (5000 ng/mL to 84 pg/mL). The assay was performed as described above. The results of the assay are shown in FIGS. 7A-7D and show that the assay can detect different GalNAc-conjugated RNAi constructs in all three serum samples from different species even though the RNAi constructs had different nucleotide sequences and patterns of chemical modifications. Also, the assay produced similar results for construct 1907 as for the other three RNAi constructs despite the 1907 construct having a different GalNAc moiety.

To explore the strand-specificity of the GalNAc intact assay, two different GalNAc-conjugated RNAi constructs and their separate sense and antisense strands were tested in mouse serum in the assay at different concentrations. The sequences and structure of the GalNAc moiety are provided in Table 6 above. The structure of the GalNAc moiety in the 1851 RNAi construct was different than that in the 8172 RNAi construct. The sequences of the sense and antisense strands of the RNAi constructs also differed. The sense strands employed in the experiment were conjugated to the GalNAc moiety, whereas the antisense strands were not conjugated to the GalNAc moiety. As shown in FIG. 8, the results of this experiment demonstrate that the GalNAc intact assay is specific for double-stranded constructs below 1 nM as the single-strand, GalNAc-conjugated sense strands were only detectable at concentrations above this threshold. As expected, no signal in the assay was observed for the single-stranded antisense strands, which lack the GalNAc moiety. These results further demonstrate the broad applicability of the assay in detecting intact GalNAc-conjugated double-strand RNA molecules irrespective of nucleotide sequence, chemical modification pattern, and structure of GalNAc moiety.

Any of the 1851 RNAi construct-specific antibodies described in Example 1, such as antibodies 17K13, 17F22, 19F24, 20P19, and 20K24, can be substituted for the pan-RNAi construct-specific antibody in the total drug assay or the intact GalNAc assay described above and depicted in FIGS. 5A and 5B to generate assays that are specific for the 1851 RNAi construct. Such assays are useful for assessing the pharmacokinetic profile, distribution, and/or metabolism of the 1851 RNAi construct specifically in subjects who have been administered the 1851 RNAi construct.

### Intact Drug Assay for Antibody-RNAi Construct Conjugate Molecules

The antibodies of the invention were incorporated into a third immunoassay to detect and quantitate intact antibody-RNAi construct conjugate molecules in biological samples. In this assay format, a pan-RNAi construct-specific antibody of the invention, such as the 14K10 antibody, is used to capture an antibody-RNAi construct conjugate molecule in a sample solution by binding to the RNA component of the conjugate molecule and immobilize the conjugate to a solid surface. The conjugate is subsequently detected and quantified using a labeled binding partner that specifically recognizes the antibody component of the conjugate molecule, such as an antibody that specifically binds to a human Fc region. This assay format is schematically shown in FIG. 5C.

Biotinylated 14K10 pan-specific RNAi construct antibody was added at a concentration of 1 μg/mL in blocking buffer (5% nonfat powdered milk in Tris buffered saline) to the wells of a streptavidin-coated gold microtiter plate (MSD GOLD™ streptavidin plate) and the plate was shaken for 30 minutes at room temperature. The plate was then washed with wash buffer (imidazole-buffered saline and Tween 20). Biological samples (serum, plasma, or tissue homogenate) were diluted 1:20 in blocking buffer and added to the wells of the microtiter plate and incubated for 1 hour at room temperature. The plate was again washed with wash buffer. Subsequently, a ruthenium-labeled mouse monoclonal antibody directed to the Fc region of human immunoglobulin (anti-human Fc antibody; 0.5 μg/mL) was added to each of the samples in blocking buffer and incubated for 1 hour at room temperature. After washing the plate with wash buffer, the signal from the ruthenium label was read using MSD Sector 5 600 electro-chemiluminescent reader and MSD Read Buffer T with surfactant.

Antibody-RNAi construct conjugate molecules were prepared by covalently attaching an RNAi construct comprising a sense strand having the sequence of SEQ ID NO: 190 and an antisense strand having the sequence of SEQ ID NO: 201 to a human monoclonal antibody (mAb) directed to a cell-surface receptor. A cysteine residue was substituted at position D70 in the light chains or at position E272 or T359 in the heavy chains of the mAb (amino acid position numbering is according to the EU numbering scheme) to create specific conjugation positions for the RNAi construct. The mAbs containing the cysteine substitutions (cys mAbs) were incubated with a solution of 2.5 mM cystamine and 2.5 mM cysteamine in 40 mM HEPES buffer, pH 7.5-8.5 for 15-20 hrs at RT and subsequently purified to provide bis-cysteamine-capped cys mAbs. The sense strand of the RNAi construct had a homoserine-aminohexanoic acid modification at its 5′ end, which was further functionalized with a bromoacetyl group using succinimidyl bromoacetate. The bis-cysteamine-capped cys mAb intermediate was partially reduced using tris(2-carboxyethyl)phosphine (TCEP) or triphenylphosphine-3,3′,3″-trisulfonic acid trisodium salt (TPPTS). Oxidation of the partially reduced cys mAb was subsequently performed with dehydroascorbic acid (DHAA), and oxidation was carried out at RT until only trace amount of reduced mAb species were observed. The bromoacetyl-RNAi construct was then added to the reaction mixture, and the alkylation was carried out at RT for 15-48 hrs. Prior to purification, 10 equivalents of N-Ethylmaleimide were added to the reaction mixture to cap any unreacted cysteines. The antibody-RNAi construct conjugate molecules with RNA-to-antibody ratio (RAR) of 1 and 2 were separated using anion exchange chromatography. A description of each of the conjugate molecules is provided in Table 7 below.

TABLE 7
Antibody-RNAi Construct Conjugate Molecules

| Molecule | Conjugation Site | RNA-to-Antibody |
|---|---|---|
| No. | in Cys mAb | Ratio |
| 15721 | D70C | 2 |
| 15722 | E272C | 1 |
| 15723 | E272C | 2 |
| 15724 | T359 | 1 |
| 15725 | T359 | 2 |

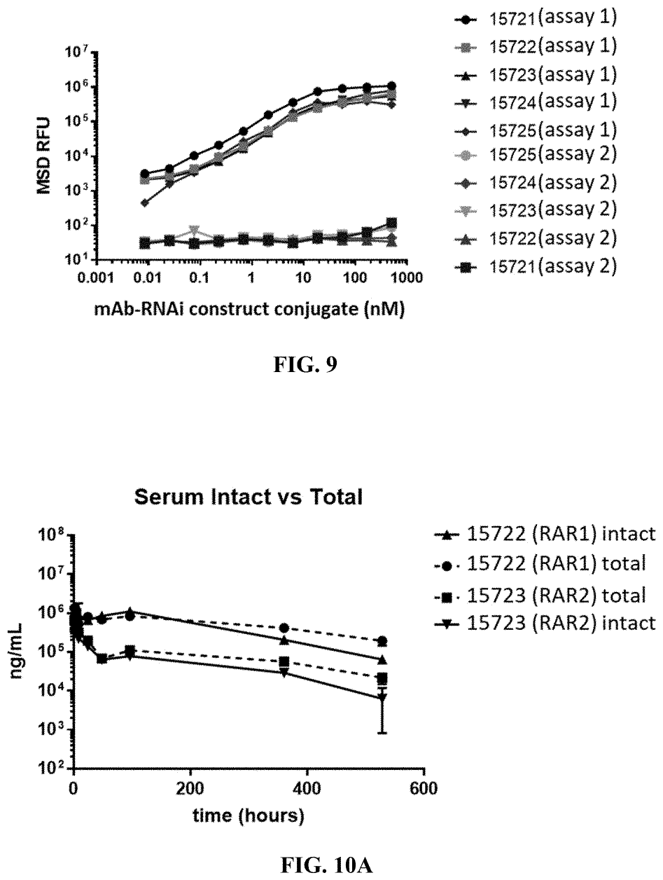

Each of the antibody-RNAi construct conjugate molecules in Table 7 was diluted in mouse serum to generate an eleven-point standard curve (500 nM to 8.5 pM) and measured using the intact drug assay for antibody-RNAi construct conjugate molecules described above and depicted in FIG. 5C (denoted as assay 1 in FIG. 9). As a negative control, the conjugate molecules were also evaluated in an immunoassay where the 14D4 anti-GalNAc moiety antibody was substituted for the 14K10 pan-specific RNAi construct antibody as the capture reagent in the assay shown in FIG. 5C (i.e. 14D4 Ab capture and an anti-human Fc antibody detect; denoted as assay 2 in FIG. 9). The results of the two assays are shown in FIG. 9. All five of the conjugates were detectable in mouse serum with the intact antibody-construct conjugate assay (14K10 Ab capture and an anti-human Fc Ab detect) and the assay was linear over most of the concentration range tested. Neither the conjugation site of the RNAi construct to the antibody nor the number of RNAi constructs conjugated to the antibody (i.e. RAR1 vs. RAR2) appeared to affect the performance of the assay. As expected, the antibody-RNAi construct conjugate molecules did not produce a signal in the immunoassay using the anti-GalNAc antibody as the capture agent (14D4 Ab capture and an anti-human Fc antibody detect) as these molecules did not contain a GalNAc moiety.

To demonstrate one application of the immunoassays of the invention, the intact antibody-RNAi construct assay described above and shown in FIG. 5C was used to analyze serum and tissue samples from mice treated with the mAb-RNAi construct conjugate molecules. C57B1/6 wild-type mice were injected intravenously with either the 15722 or 15723 mAb-RNAi conjugate molecule described in Table 7 at a dose of 12 mg/kg. The only difference between the 15722 conjugate and the 15723 conjugate was the number of RNAi constructs conjugated to the antibody; the 15722 conjugate contained one RNAi construct (i.e. RAR1), whereas the 15723 conjugate contained two RNAi construct molecules (i.e. RAR2). Serum was collected from the animals 5 min, 15 min, 30 min, and 1, 2, 4, 8, 24, 96, 360, and 528 hours following conjugate molecule administration. Tissues, including liver, pancreas, and kidney, were collected from the animals at 5 min, 15 min, and 4, 8, 24, 96, 360, and 528 hours following conjugate molecule administration.

The intact assay method for antibody-RNAi construct conjugate molecules described above was used to measure the amount of intact mAb-RNAi construct conjugate molecules in the serum and tissue samples collected from the animals treated with the conjugate molecules. The samples were diluted 1:20 in blocking buffer and added to the wells of a streptavidin-coated gold microtiter plate containing biotinylated 14K10 pan-specific RNAi construct antibody and incubated for 1 hour at room temperature. Following washing of the plate with wash buffer as described above, detection of the captured conjugate molecules was achieved using the ruthenium-labeled anti-human Fc mAb and the electro-chemiluminescent signal was read by an MSD Sector 5 600 electro-chemiluminescent reader. As a measure of total conjugate molecule in the serum and tissue samples, an anti-Fc/anti-Fc sandwich ELISA assay was employed. In the total conjugate molecule ELISA assay, a first biotinylated anti-human Fc antibody was used to capture any human mAb in the sample and a second ruthenium-labeled anti-human Fc antibody binding to a different epitope than the first anti-human Fc antibody was used to detect captured human mAb. The total conjugate molecule assay will detect naked human mAbs (i.e. mAbs that have lost the RNAi constructs) as well as mAbs with one or two linked RNAi constructs.

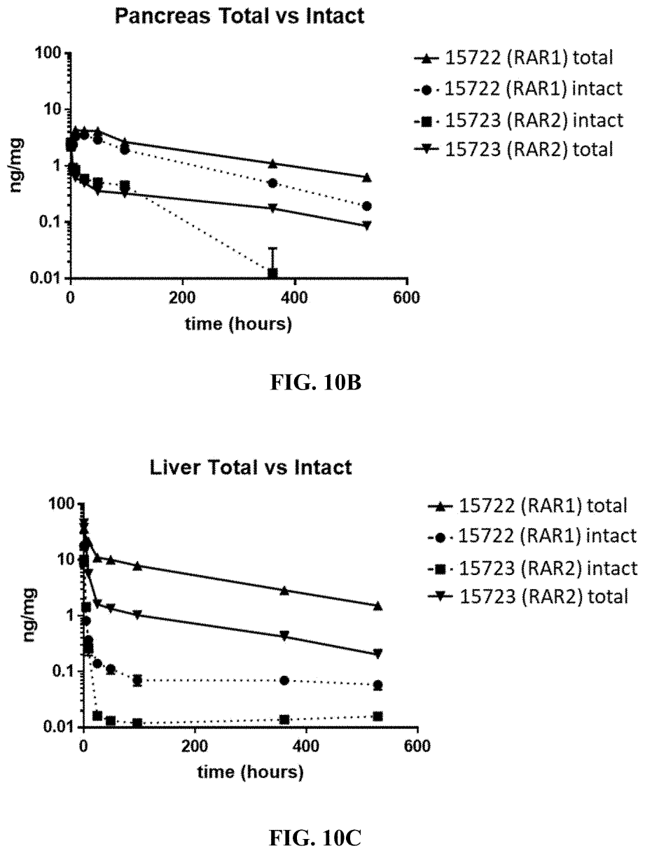

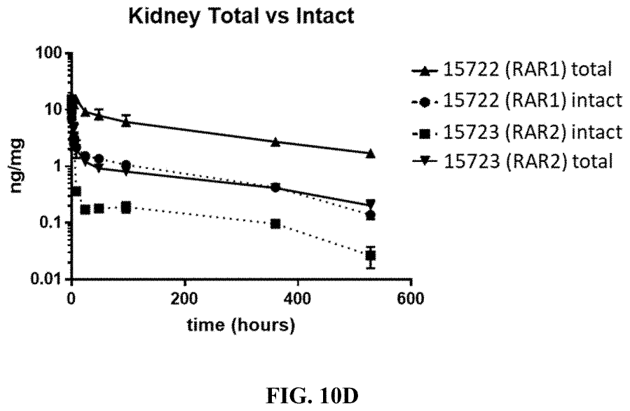

The results from the analysis of the serum samples are shown in FIG. 10A and the results from the analysis of the pancreas, liver, and kidney samples are shown in FIGS. 10B to 10D, respectively. Both conjugate molecules remained intact in the blood compartment for extended periods of time (FIG. 10A). The conjugate molecules also remained intact for prolonged periods of time in the pancreas, with the intact form of the RAR1 conjugate persisting longer than the intact form of the RAR2 conjugate (FIG. 10B). The conjugate molecules rapidly lost the RNAi construct components in liver and kidney (FIGS. 10C and 10D). These experimental results demonstrate that the assay methods and antibodies of the invention can be employed in pharmacokinetic and drug metabolism studies to assess the clearance profile and metabolic degradation of antibody-RNAi construct conjugate molecules in vivo.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

1. A method for generating a monoclonal antibody that specifically binds to a chemically-modified nucleic acid molecule comprising:
(a) conjugating a plurality of nucleic acid molecules to a bead to form an immunogen, wherein the nucleic acid molecules each comprise one or more modified nucleotides;
(b) administering the immunogen to an animal;
(c) obtaining splenocytes from the immunized animal;
(d) selecting splenocytes that are IgG positive and bind to the chemically-modified nucleic acid molecule thereby isolating antigen-specific antibody producing cells;
(e) plating the antigen-specific antibody producing cells in single-cell culture; and
(f) isolating the monoclonal antibody from the single-cell culture.

2. The method of claim 1, wherein the bead has an average diameter of at least 70 nm.

3. The method of claim 1, wherein the bead has an average diameter of about 50 nm to about 2 μm.

4. The method of claim 1, wherein the nucleic acid molecules are double-stranded.

5. The method of claim 4, wherein the nucleic acid molecules are RN-Ai constructs each comprising a sense strand and an anti sense strand.

6. The method of claim 5, wherein the sense strand and the antisense strand are each independently about 19 to about 30 nucleotides in length.

7. The method of claim 1, wherein the nucleic acid molecules are single-stranded.

8. The method of claim 1, wherein the nucleic acid molecules each comprise one or more modified nucleotides selected from 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNA), or combinations thereof.

9. The method of claim 1, wherein each of the nucleic acid molecules is covalently linked to a ligand comprising a carbohydrate.

10. The method of claim 9, wherein the carbohydrate is galactose, galactosamine, or N-acetyl-galactosamine.

11. The method of claim 10, wherein the ligand comprises a multivalent galactose moiety or multivalent N-acetyl-galactosamine moiety.

12. The method of claim 11, wherein the multivalent galactose moiety or multivalent N-acetyl-galactosamine moiety is trivalent or tetravalent.

13. The method of claim 1, wherein the animal to be administered the immunogen is a rabbit.

14. (canceled)

15. An isolated monoclonal antibody that specifically binds to a chemically-modified nucleic acid molecule independent of nucleotide sequence, wherein the monoclonal antibody comprises (i) a light chain variable region comprising complementarity determining regions CDRL1, CDRL2, and, CDRL3, and (ii) a heavy chain variable region comprising complementarity determining regions CDRH1, CDRH2, and CDRH3, and wherein:
(a) CDRL1, CDRL2, and CDRL3 have the sequence of SEQ NOs: 1, 14, and 25, respectively, and CDRH1, CDRH2, and CDRH3 have the sequence of SEQ ID NOs: 51, 64, and 77, respectively;
(b) CDRL1, CDRL2, and CDRL3 have the sequence of SEQ ID NOs: 2, 15, and 26, respectively, and CDRH1, CDRH2, and CDRH3 have the sequence of SEQ ID NOs: 52, 65, and 78, respectively; or
(c) CDRL1, CDRL2, and CDRL3 have the sequence of SEQ ID NOs: 3, 16, and 27, respectively, and CDRL1, CDRH2, and CDRH3 have the sequence of SEQ ID NOs: 53, 66, and 79, respectively.

16. (canceled)

17. An isolated monoclonal antibody that binds in a sequence specific manner to an RNAi construct comprising the nucleotide sequence of SEQ ID NO: 192, wherein the monoclonal antibody comprises (i) a light chain variable region comprising complementarity determining regions CDRL1, CDRL2, and CDRL3, and (ii) a heavy chain variable region comprising complementarity determining regions CDRH1, CDRH2, and CDRH3, and wherein:
(a) CDRL1, CDRL2, and CDRL3 have the sequence of SEQ ID NOs: 4, 17, and 28, respectively, and CDRH1, CDRH2, and CDRH3 have the sequence of SEQ ID NOs: 54, 67, and 80, respectively;
(b) CDRL1, CDRL2, and CDRL3 have the sequence of SEQ ID NOs: 5, 18, and 29, respectively, and CDRH1, CDRH2, and CDRH3 have the sequence of SEQ ID-NOs: 55, 68, and 81, respectively;
(c) CDRL1, CDRL2, and CDRL3 have the sequence of SEQ NOs: 6, 19, and 30, respectively, and CDRH1, CDRH2, and CDRH3 have the sequence of SEQ ID NOs: 56, 69, and 82, respectively;
(d) CDRL1, CDRL2, and CDRL3 have the sequence of SEQ ID NOs: 7, 20, and 31, respectively, and CDRH1, CDRH2, and CDRH3 have the sequence of SEQ ID NOs: 57, 70, and 83, respectively; or
(e) CDRL2, and CDRL3 have the sequence of SEQ NOs: 8, 17, and 32, respectively, and CDRH1, CDRH2, and CDRH3 have the sequence of SEQ ID NOs: 58, 71, and 84, respectively.

18. (canceled)

19. An isolated monoclonal antibody that specifically binds to a N-acetyl-galactosamine (GalNAc) moiety, wherein the monoclonal antibody comprises (i) a light chain variable region comprising complementarity determining regions CDRL1, CDRL2, and CDRL3, and (ii) a heavy chain variable region comprising complementarity determining regions CDRH1, CDRH2, and CDRH3, and wherein:
(a) CDRL1, CDRL2, and CDRL3 have the sequence of SEQ ID NOs: 9, 21, and 33, respectively, and CDRH1, CDRH2, and CDRH3 have the sequence of SEQ ID-NOs: 59, 72, and 85, respectively;
(b) CDRL1, CDRL2, and CDRL3 have the sequence of SEQ NOs: 10, 22, and 34, respectively, and CDRH1 CDRH2, and CDRH3 have the sequence of SEQ ID NOs: 60, 73, and 86, respectively;
(c) CDRL1, CDRL2, and CDRL3 have the sequence of SEQ ID NOs: 11, 19, and 35, respectively, and CDRH1, CDRH2, and CDRH3 have the sequence of SEQ ID NOs: 61, 74, and 87, respectively;
(d) CDRL2, and CDRL3 have the sequence of SEQ ID NOs: 12, 23, and 36, respectively, and CDRH1, CDRH2, and CDRH3 have the sequence of SEQ ID-NOs: 62, 75, and 88, respectively; or
(e) CDRL2, and CDRL3 have the sequence of SEQ NOs: 13, 24, and 37, respectively, and CDRH1, CDRH2, and CDRH3 have the sequence of SEQ ID NOs: 63, 76, and 89, respectively.

20-23. (canceled)

24. A method for detecting a chemically-modified nucleic acid molecule in a sample comprising:
(a) providing a surface comprising a capture antibody that specifically binds to the chemically-modified nucleic acid molecule, wherein the capture antibody is any one of the monoclonal antibodies of claim 15;
(b) contacting the surface with the sample under conditions allowing the chemically-modified nucleic acid molecule, if present in the sample, to bind to the capture antibody on the surface;
(c) contacting the surface with a detection reagent, wherein the detection reagent comprises a detectable label coupled to a binding partner that specifically binds to the chemically-modified nucleic acid molecule; and
(d) detecting a signal from the detectable label.

25. The method of claim 24, wherein the binding partner is a second antibody that specifically binds to the chemically-modified nucleic acid molecule.

26-32. (canceled)

33. The method of claim 24, wherein the detectable label is a fluorophore, metallic nanoparticle, enzyme, radiolabel, or ECL luminophore.

34-39. (canceled)

40. A kit for detecting a chemically-modified nucleic acid molecule in a sample comprising:
(a) a capture antibody immobilized to a surface, wherein the capture antibody specifically binds to the chemically-modified nucleic acid molecule and is any one of the monoclonal antibodies of claim 15;
(b) a detection reagent comprising a detectable label coupled to a binding partner that specifically binds to the chemically-modified nucleic acid molecule; and
(c) instructions for contacting the sample with the immobilized capture antibody and detection reagent, and instructions for detecting a signal from the detectable label.

41-46. (canceled)


SEQUENCE LISTING (202 sequences)

SEQ ID NO: 1 - protein, 13 aa, Artificial Sequence
  synthetic polypeptide
QASQSVLNNNYLS

SEQ ID NO: 2 - protein, 12 aa, Artificial Sequence
  synthetic polypeptide
QSSLSVNRSDLS

SEQ ID NO: 3 - protein, 13 aa, Artificial Sequence
  synthetic polypeptide
QASQSVANNNYLA

SEQ ID NO: 4 - protein, 13 aa, Artificial Sequence
  synthetic polypeptide
QSSQSVWQKNWLS

SEQ ID NO: 5 - protein, 13 aa, Artificial Sequence
  synthetic polypeptide
QASQSVYNNNNLA

SEQ ID NO: 6 - protein, 11 aa, Artificial Sequence
  synthetic polypeptide
QASESISSWLA

SEQ ID NO: 7 - protein, 11 aa, Artificial Sequence
  synthetic polypeptide
QASQSINNELA

SEQ ID NO: 8 - protein, 13 aa, Artificial Sequence
  synthetic polypeptide
QSSQSVWEKNWLS

SEQ ID NO: 9 - protein, 13 aa, Artificial Sequence
  synthetic polypeptide
QSSESVYENNDLS

SEQ ID NO: 10 - protein, 13 aa, Artificial Sequence
  synthetic polypeptide
QASQSLYKNTDLA

SEQ ID NO: 11 - protein, 13 aa, Artificial Sequence
  synthetic polypeptide
QSSQSVGNNAYLS

SEQ ID NO: 12 - protein, 12 aa, Artificial Sequence
  synthetic polypeptide
QASQDLYSNCLS

SEQ ID NO: 13 - protein, 11 aa, Artificial Sequence
  synthetic polypeptide
QASQSINSWLS

SEQ ID NO: 14 - protein, 7 aa, Artificial Sequence
  synthetic polypeptide
SASKLAT

SEQ ID NO: 15 - protein, 7 aa, Artificial Sequence
  synthetic polypeptide
LASNLES

SEQ ID NO: 16 - protein, 7 aa, Artificial Sequence
  synthetic polypeptide
QASTLAS

SEQ ID NO: 17 - protein, 7 aa, Artificial Sequence
  synthetic polypeptide
RASTLAS

SEQ ID NO: 18 - protein, 7 aa, Artificial Sequence
  synthetic polypeptide
YTSTLAS

SEQ ID NO: 19 - protein, 7 aa, Artificial Sequence
  synthetic polypeptide
YASTLAS

SEQ ID NO: 20 - protein, 7 aa, Artificial Sequence
  synthetic polypeptide
LAFTLAS

SEQ ID NO: 21 - protein, 7 aa, Artificial Sequence
  synthetic polypeptide
WASSLAS

SEQ ID NO: 22 - protein, 7 aa, Artificial Sequence
  synthetic polypeptide
FASNLAS

SEQ ID NO: 23 - protein, 7 aa, Artificial Sequence
  synthetic polypeptide
LTSTLAS

SEQ ID NO: 24 - protein, 7 aa, Artificial Sequence
  synthetic polypeptide
AASTLAS

SEQ ID NO: 25 - protein, 12 aa, Artificial Sequence
  synthetic polypeptide
AGYKNFGNDDNA

SEQ ID NO: 26 - protein, 12 aa, Artificial Sequence
  synthetic polypeptide
AGGYSSGNDRNA

SEQ ID NO: 27 - protein, 12 aa, Artificial Sequence
  synthetic polypeptide
AGYRSYINADNA

SEQ ID NO: 28 - protein, 12 aa, Artificial Sequence
  synthetic polypeptide
AGAYSSNSDVRA

SEQ ID NO: 29 - protein, 13 aa, Artificial Sequence
  synthetic polypeptide
QGEFACSTADCLT

SEQ ID NO: 30 - protein, 12 aa, Artificial Sequence
  synthetic polypeptide
AGYGSANDDKNG

SEQ ID NO: 31 - protein, 12 aa, Artificial Sequence
  synthetic polypeptide
QQGYIISGVDNV

SEQ ID NO: 32 - protein, 12 aa, Artificial Sequence
  synthetic polypeptide
AGAYSVNSDVRA

SEQ ID NO: 33 - protein, 12 aa, Artificial Sequence
  synthetic polypeptide
AGYKSMSTDGFA

SEQ ID NO: 34 - protein, 12 aa, Artificial Sequence
  synthetic polypeptide
AGYKSSTTDGFG

SEQ ID NO: 35 - protein, 10 aa, Artificial Sequence
  synthetic polypeptide
QAYYHVGVAA

SEQ ID NO: 36 - protein, 11 aa, Artificial Sequence
  synthetic polypeptide
QGFHGYGVGAA

SEQ ID NO: 37 - protein, 12 aa, Artificial Sequence
  synthetic polypeptide
QGYDGSSGSAAS

SEQ ID NO: 38 - protein, 112 aa, Artificial Sequence
  synthetic polypeptide
ALVMTQTPSPVSAAVGGTVTINCQASQSVLNNNYLSWYQQKPGQPPKLLIYSASKLATGVPSRFSGSGSGTQFTLTISGVQCDDAATYYCAGYKNFGNDDNAFGGGTEVVVK

SEQ ID NO: 39 - protein, 111 aa, Artificial Sequence
  synthetic polypeptide
AQVLTQTSSPVSVNMGGTVTINCQSSLSVNRSDLSWYQQKPGQPPKLLIYLASNLESGVPSRFKGSGSGTHFTLTINGVECDDAATYYCAGGYSSGNDRNAFGGGTEVVVK

SEQ ID NO: 40 - protein, 112 aa, Artificial Sequence
  synthetic polypeptide
ALVMTQTPSPVSAAVGGTVTISCQASQSVANNNYLAWYQQKPGQPPKLLIYQASTLASGVPSRFKGSGSGTQFTLTISGVECDDAATYYCAGYRSYINADNAFGGGTEVVVE

SEQ ID NO: 41 - protein, 112 aa, Artificial Sequence
  synthetic polypeptide
AQVLTQTPSSVSAAVGGTVTINCQSSQSVWQKNWLSWFQQKPGQPPKLLIYRASTLASGVPSRFKSSGSGTQFTLTISGVQCDDAATYYCAGAYSSNSDVRAFGGGTELVVK

SEQ ID NO: 42 - protein, 113 aa, Artificial Sequence
  synthetic polypeptide
AQVLTQTASPVSAAVGSTVTINCQASQSVYNNNNLAWYQQKPGQPPKLLIYYTSTLASGVSSRFKGSGSGTQFTLTISGMQCDDAATYYCQGEFACSTADCLTFGGGTEVVVK

SEQ ID NO: 43 - protein, 110 aa, Artificial Sequence
  synthetic polypeptide
ALVMTQTPASVSAAVGGTVTINCQASESISSWLAWYQQKPGQPPKLLIYYASTLASGVSSRFKGSGSRTEYTLTISDLECADAATYYCAGYGSANDDKNGFGGGTEVVVK

SEQ ID NO: 44 - protein, 110 aa, Artificial Sequence
  synthetic polypeptide
AYDMTQTPASVEVAVGGTVTIKCQASQSINNELAWYQQKPGQPPKLLIYLAFTLASGVPSRFKGSRSGTEFTLTISDLECADAATYYCQQGYIISGVDNVFGGGTEVVVK

SEQ ID NO: 45 - protein, 112 aa, Artificial Sequence
  synthetic polypeptide
AQVLTQTPSSVSAAVGGTVTINCQSSQSVWEKNWLSWFQQKPGQPPKLLIYRASTLASGVPSRFKSSGSGTQFTLTISGVQCDDAAVYYCAGAYSVNSDVRAFGGGTELVVK

SEQ ID NO: 46 - protein, 112 aa, Artificial Sequence
  synthetic polypeptide
AIVMTQTPSSKSVPVGGTVTINCQSSESVYENNDLSWYQQKPGQPPKLLIYWASSLASGVPSRFEGSGSGTQFTLTISNVVCDDAATYYCAGYKSMSTDGFAFGGGTEVVVK

SEQ ID NO: 47 - protein, 112 aa, Artificial Sequence
  synthetic polypeptide
AIVMTQTPSSKSVPVGGTVTISCQASQSLYKNTDLAWFQQKPGQPPKLLIYFASNLASGVTSRFKGSGSGTQFTLTISDVVCDDAATYYCAGYKSSTTDGFGFGGGTEVVVK

SEQ ID NO: 48 - protein, 110 aa, Artificial Sequence
  synthetic polypeptide
AQVLAQTPSSVSAAVGGTVTIDCQSSQSVGNNAYLSWYQQKPGQPPKLLIYYASTLASGVPSRFSGSGSGTHFTLTISGVQCDDAATYYCQAYYHVGVAAFGGGTEVVVK

SEQ ID NO: 49 - protein, 110 aa, Artificial Sequence
  synthetic polypeptide
DVVMTQTPASVSESVGGTVTIKCQASQDLYSNCLSWYQQKPGQRPKLLMYLTSTLASGVPSRFKGSGSGTDFTLTISDLECADAATYYCQGFHGYGVGAAFGGGTEVVVK

SEQ ID NO: 50 - protein, 110 aa, Artificial Sequence
  synthetic polypeptide
DVVMTQTPASVEAAVGGTVTIKCQASQSINSWLSWYQQKPGQRPKLLIYAASTLASGVSSRFKGSKSGTEFTLTISGVQCDDAATYYCQGYDGSSGSAASFGGGTEVVVK

SEQ ID NO: 51 - protein, 6 aa, Artificial Sequence
  synthetic polypeptide
SSYYMW

SEQ ID NO: 52 - protein, 5 aa, Artificial Sequence
  synthetic polypeptide
GHYMS

SEQ ID NO: 53 - protein, 6 aa, Artificial Sequence
  synthetic polypeptide
SNYYMC

SEQ ID NO: 54 - protein, 6 aa, Artificial Sequence
  synthetic polypeptide
GSYWIN

SEQ ID NO: 55 - protein, 5 aa, Artificial Sequence
  synthetic polypeptide
SYFMS

SEQ ID NO: 56 - protein, 6 aa, Artificial Sequence
  synthetic polypeptide
SGYYMC

SEQ ID NO: 57 - protein, 6 aa, Artificial Sequence
  synthetic polypeptide
NNYIMC

SEQ ID NO: 58 - protein, 6 aa, Artificial Sequence
  synthetic polypeptide
SSYWIN

SEQ ID NO: 59 - protein, 5 aa, Artificial Sequence
  synthetic polypeptide
RYVVD

SEQ ID NO: 60 - protein, 5 aa, Artificial Sequence
  synthetic polypeptide
YFGMY

SEQ ID NO: 61 - protein, 5 aa, Artificial Sequence
  synthetic polypeptide
SYDMG

SEQ ID NO: 62 - protein, 5 aa, Artificial Sequence
  synthetic polypeptide
TNAMT

SEQ ID NO: 63 - protein, 5 aa, Artificial Sequence
  synthetic polypeptide
DHYMS

SEQ ID NO: 64 - protein, 18 aa, Artificial Sequence
  synthetic polypeptide
CINGGSRGTTYYASWAKG

SEQ ID NO: 65 - protein, 17 aa, Artificial Sequence
  synthetic polypeptide
HIYGSGRGLWYANWAKG

SEQ ID NO: 66 - protein, 18 aa, Artificial Sequence
  synthetic polypeptide
CIYAGSSGSTYYASWAKG

SEQ ID NO: 67 - protein, 16 aa, Artificial Sequence
  synthetic polypeptide
IIAAGGRIWYASSVKG

SEQ ID NO: 68 - protein, 16 aa, Artificial Sequence
  synthetic polypeptide
IIYGRDKTYYATWTKG

SEQ ID NO: 69 - protein, 18 aa, Artificial Sequence
  synthetic polypeptide
CIYAGSGARTYYASWAKG

SEQ ID NO: 70 - protein, 17 aa, Artificial Sequence
  synthetic polypeptide
CISTLTTATYYATWAKG

SEQ ID NO: 71 - protein, 16 aa, Artificial Sequence
  synthetic polypeptide
IMPAGGRPYYATWAKG

SEQ ID NO: 72 - protein, 15 aa, Artificial Sequence
  synthetic polypeptide
TIGYGSTWYASWVKG

SEQ ID NO: 73 - protein, 16 aa, Artificial Sequence
  synthetic polypeptide
TIDSSDIIYYASWAKG

SEQ ID NO: 74 - protein, 16 aa, Artificial Sequence
  synthetic polypeptide
YIFINDNTYYATWAKG

SEQ ID NO: 75 - protein, 17 aa, Artificial Sequence
  synthetic polypeptide
YIYEGSGNTFYASWAKG

SEQ ID NO: 76 - protein, 16 aa, Artificial Sequence
  synthetic polypeptide
YISEGGATYYASWAKG

SEQ ID NO: 77 - protein, 12 aa, Artificial Sequence
  synthetic polypeptide
DPYGFSGSIYAL

SEQ ID NO: 78 - protein, 10 aa, Artificial Sequence
  synthetic polypeptide
YRPVDYVMDI

SEQ ID NO: 79 - protein, 12 aa, Artificial Sequence
  synthetic polypeptide
ERENYIGVGYYL

SEQ ID NO: 80 - protein, 10 aa, Artificial Sequence
  synthetic polypeptide
DDIGIPGGDI

SEQ ID NO: 81 - protein, 15 aa, Artificial Sequence
  synthetic polypeptide
SSSSGRGLYYGGMDP

SEQ ID NO: 82 - protein, 13 aa, Artificial Sequence
  synthetic polypeptide
DPYSVNDPVGDTL

SEQ ID NO: 83 - protein, 13 aa, Artificial Sequence
  synthetic polypeptide
DGWSGDGVITFNL

SEQ ID NO: 84 - protein, 10 aa, Artificial Sequence
  synthetic polypeptide
DDIGTPGGDI

SEQ ID NO: 85 - protein, 10 aa, Artificial Sequence
  synthetic polypeptide
GNVGSTGVSI

SEQ ID NO: 86 - protein, 10 aa, Artificial Sequence
  synthetic polypeptide
SGGVAGGDSV

SEQ ID NO: 87 - protein, 8 aa, Artificial Sequence
  synthetic polypeptide
GYFGGMDP

SEQ ID NO: 88 - protein, 8 aa, Artificial Sequence
  synthetic polypeptide
GYLGAMDP

SEQ ID NO: 89 - protein, 8 aa, Artificial Sequence
  synthetic polypeptide
GWLAAFDP

SEQ ID NO: 90 - protein, 121 aa, Artificial Sequence
  synthetic polypeptide
QSLEESGGDLVKPGASLTLTCTASGFSFSSSYYMWWVRQAPGKGLEWIACINGGSRGTTYYASWAKGRFTISKTSSTTVTLQMTSLTVADTATYFCARDPYGFSGSIYALWGPGTLVTVSS

SEQ ID NO: 91 - protein, 116 aa, Artificial Sequence
  synthetic polypeptide
QSLEESGGRLVTPGTPLTLSCKASGLSLSGHYMSWVRQAPGKGLEWIGHIYGSGRGLWYANWAKGRFTISKTSTTVDLKIISPTNEDTATYFCARYRPVDYVMDIWGPGTLVTVSL

SEQ ID NO: 92 - protein, 122 aa, Artificial Sequence
  synthetic polypeptide
QEQLEESGGDLVKPEGSLTLTCTASGFSFSSNYYMCWVRQAPGKGLEWIACIYAGSSGSTYYASWAKGRFTVSKTSSTTVTLQMTSLTAADTATYFCARERENYIGVGYYLWGPGTLVTVSS

SEQ ID NO: 93 - protein, 116 aa, Artificial Sequence
  synthetic polypeptide
EQLKESGGRLVTPGTPLTLTCTVSGFSLSGSYWINWVRQAPGKGLEWIAIIAAGGRIWYASSVKGRFTISKTSTTVDLKMTSPTTEDTATYFCARDDIGIPGGDIWGPGTLVTVSL

SEQ ID NO: 94 - protein, 121 aa, Artificial Sequence
  synthetic polypeptide
QEQLKESGGGLVTPGGTLTLTCTASGFTINSYFMSWVRQAPGKGLEWIGIIYGRDKTYYATWTKGRFTISKTSTTVDLIITSPTTEDTATYFCARSSSSGRGLYYGGMDPWGPGTLVTVSS

SEQ ID NO: 95 - protein, 122 aa, Artificial Sequence
  synthetic polypeptide
QSLEESGGDLVKPGASLTLTCTASGFSFSSGYYMCWVRQAPGKGLEWIACIYAGSGARTYYASWAKGRFTISKTSSTTVTLQMTSLTAADTATYFCARDPYSVNDPVGDTLWGPGTLVTVSS

SEQ ID NO: 96 - protein, 121 aa, Artificial Sequence
  synthetic polypeptide
ERLEESGGDLVKPGASLTLTCKASGADFNNNYIMCWVRQAPGKGLEWIACISTLTTATYYATWAKGRFTISTTSSTTVTLQMTSLTAADTATYFCAGDGWSGDGVITFNLWGPGTLVTVSS

SEQ ID NO: 97 - protein, 116 aa, Artificial Sequence
  synthetic polypeptide
QSLEESGGRLVTPGTPLTLTCTASGFSLSSSYWINWVRQAPGKGLEWIAIMPAGGRPYYATWAKGRFIISKTSTTVDLKMTSPTTEDTATYFCARDDIGTPGGDIWGPGTLVTVSL

SEQ ID NO: 98 - protein, 114 aa, Artificial Sequence
  synthetic polypeptide
QSVEESGGRLVTPGTPLTLTCTVSRIDLSRYVVDWVRQAPGEGLEWIGTIGYGSTWYASWVKGRFTISRTSTTVDLKMTSLTTEDTATYFCARGNVGSTGVSIWGPGTLVTVSL

SEQ ID NO: 99 - protein, 115 aa, Artificial Sequence
  synthetic polypeptide
QSVEESGGRLVAPGTPLTLTCTVSGFSLSYFGMYWVRQAPGRGLEWIGTIDSSDIIYYASWAKGRFTISKTSTTVDLKITSPTTEDTATYFCARSGGVAGGDSVWGPGTLVTVSL

SEQ ID NO: 100 - protein, 113 aa, Artificial Sequence
  synthetic polypeptide
QSLEESGGRLVTPGGYLTLTCTVSGFSLSSYDMGWVRQAPGKGLEWIGYIFINDNTYYATWAKGRFTISKTSTTMDLKMTSLTTEDTATYFCVRGYFGGMDPWGPGTLVTVSS

SEQ ID NO: 101 - protein, 114 aa, Artificial Sequence
  synthetic polypeptide
QSVEESGGRLVTPGASLTLTCTVSGIDFSTNAMTWVRQAPGKGLEWIGYIYEGSGNTFYASWAKGRFTISRTSTTVDLKMTSLTMEDTATYFCARGYLGAMDPWGPGTLVTVSS

SEQ ID NO: 102 - protein, 114 aa, Artificial Sequence
  synthetic polypeptide
QSVEESGGGLVKPGASLTLTCQVSGFSLSDHYMSWVRQAPGKGLEWVAYISEGGATYYASWAKGRFTISKTSSTTVDLKMTSLTTEDTATYFCARGWLAAFDPWGPGTLVTVSS

SEQ ID NO: 103 - protein, 216 aa, Artificial Sequence
  synthetic polypeptide
ALVMTQTPSPVSAAVGGTVTINCQASQSVLNNNYLSWYQQKPGQPPKLLIYSASKLATGVPSRFSGSGSGTQFTLTISGVQCDDAATYYCAGYKNFGNDDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 104 - protein, 215 aa, Artificial Sequence
  synthetic polypeptide
AQVLTQTSSPVSVNMGGTVTINCQSSLSVNRSDLSWYQQKPGQPPKLLIYLASNLESGVPSRFKGSGSGTHFTLTINGVECDDAATYYCAGGYSSGNDRNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 105 - protein, 216 aa, Artificial Sequence
  synthetic polypeptide
ALVMTQTPSPVSAAVGGTVTISCQASQSVANNNYLAWYQQKPGQPPKLLIYQASTLASGVPSRFKGSGSGTQFTLTISGVECDDAATYYCAGYRSYINADNAFGGGTEVVVEGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 106 - protein, 216 aa, Artificial Sequence
  synthetic polypeptide
AQVLTQTPSSVSAAVGGTVTINCQSSQSVWQKNWLSWFQQKPGQPPKLLIYRASTLASGVPSRFKSSGSGTQFTLTISGVQCDDAATYYCAGAYSSNSDVRAFGGGTELVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 107 - protein, 217 aa, Artificial Sequence
  synthetic polypeptide
AQVLTQTASPVSAAVGSTVTINCQASQSVYNNNNLAWYQQKPGQPPKLLIYYTSTLASGVSSRFKGSGSGTQFTLTISGMQCDDAATYYCQGEFACSTADCLTFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 108 - protein, 214 aa, Artificial Sequence
  synthetic polypeptide
ALVMTQTPASVSAAVGGTVTINCQASESISSWLAWYQQKPGQPPKLLIYYASTLASGVSSRFKGSGSRTEYTLTISDLECADAATYYCAGYGSANDDKNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 109 - protein, 214 aa, Artificial Sequence
  synthetic polypeptide
AYDMTQTPASVEVAVGGTVTIKCQASQSINNELAWYQQKPGQPPKLLIYLAFTLASGVPSRFKGSRSGTEFTLTISDLECADAATYYCQQGYIISGVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 110 - protein, 216 aa, Artificial Sequence
  synthetic polypeptide
AQVLTQTPSSVSAAVGGTVTINCQSSQSVWEKNWLSWFQQKPGQPPKLLIYRASTLASGVPSRFKSSGSGTQFTLTISGVQCDDAAVYYCAGAYSVNSDVRAFGGGTELVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 111 - protein, 216 aa, Artificial Sequence
  synthetic polypeptide
AIVMTQTPSSKSVPVGGTVTINCQSSESVYENNDLSWYQQKPGQPPKLLIYWASSLASGVPSRFEGSGSGTQFTLTISNVVCDDAATYYCAGYKSMSTDGFAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 112 - protein, 216 aa, Artificial Sequence
  synthetic polypeptide
AIVMTQTPSSKSVPVGGTVTISCQASQSLYKNTDLAWFQQKPGQPPKLLIYFASNLASGVTSRFKGSGSGTQFTLTISDVVCDDAATYYCAGYKSSTTDGFGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 113 - protein, 214 aa, Artificial Sequence
  synthetic polypeptide
AQVLAQTPSSVSAAVGGTVTIDCQSSQSVGNNAYLSWYQQKPGQPPKLLIYYASTLASGVPSRFSGSGSGTHFTLTISGVQCDDAATYYCQAYYHVGVAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 114 - protein, 214 aa, Artificial Sequence
  synthetic polypeptide
DVVMTQTPASVSESVGGTVTIKCQASQDLYSNCLSWYQQKPGQRPKLLMYLTSTLASGVPSRFKGSGSGTDFTLTISDLECADAATYYCQGFHGYGVGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 115 - protein, 214 aa, Artificial Sequence
  synthetic polypeptide
DVVMTQTPASVEAAVGGTVTIKCQASQSINSWLSWYQQKPGQRPKLLIYAASTLASGVSSRFKGSKSGTEFTLTISGVQCDDAATYYCQGYDGSSGSAASFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 116 - protein, 444 aa, Artificial Sequence
  synthetic polypeptide
QSLEESGGDLVKPGASLTLTCTASGFSFSSSYYMWWVRQAPGKGLEWIACINGGSRGTTYYASWAKGRFTISKTSSTTVTLQMTSLTVADTATYFCARDPYGFSGSIYALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 117 - protein, 439 aa, Artificial Sequence
  synthetic polypeptide
QSLEESGGRLVTPGTPLTLSCKASGLSLSGHYMSWVRQAPGKGLEWIGHIYGSGRGLWYANWAKGRFTISKTSTTVDLKIISPTNEDTATYFCARYRPVDYVMDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 118 - protein, 445 aa, Artificial Sequence
  synthetic polypeptide
QEQLEESGGDLVKPEGSLTLTCTASGFSFSSNYYMCWVRQAPGKGLEWIACIYAGSSGSTYYASWAKGRFTVSKTSSTTVTLQMTSLTAADTATYFCARERENYIGVGYYLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 119 - protein, 439 aa, Artificial Sequence
  synthetic polypeptide
EQLKESGGRLVTPGTPLTLTCTVSGFSLSGSYWINWVRQAPGKGLEWIAIIAAGGRIWYASSVKGRFTISKTSTTVDLKMTSPTTEDTATYFCARDDIGIPGGDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 120 - protein, 444 aa, Artificial Sequence
  synthetic polypeptide
QEQLKESGGGLVTPGGTLTLTCTASGFTINSYFMSWVRQAPGKGLEWIGIIYGRDKTYYATWTKGRFTISKTSTTVDLIITSPTTEDTATYFCARSSSSGRGLYYGGMDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 121 - protein, 445 aa, Artificial Sequence
  synthetic polypeptide
QSLEESGGDLVKPGASLTLTCTASGFSFSSGYYMCWVRQAPGKGLEWIACIYAGSGARTYYASWAKGRFTISKTSSTTVTLQMTSLTAADTATYFCARDPYSVNDPVGDTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 122 - protein, 444 aa, Artificial Sequence
  synthetic polypeptide
ERLEESGGDLVKPGASLTLTCKASGADFNNNYIMCWVRQAPGKGLEWIACISTLTTATYYATWAKGRFTISTTSSTTVTLQMTSLTAADTATYFCAGDGWSGDGVITFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 123 - protein, 439 aa, Artificial Sequence
  synthetic polypeptide
QSLEESGGRLVTPGTPLTLTCTASGFSLSSSYWINWVRQAPGKGLEWIAIMPAGGRPYYATWAKGRFIISKTSTTVDLKMTSPTTEDTATYFCARDDIGTPGGDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 124 - protein, 437 aa, Artificial Sequence
  synthetic polypeptide
QSVEESGGRLVTPGTPLTLTCTVSRIDLSRYVVDWVRQAPGEGLEWIGTIGYGSTWYASWVKGRFTISRTSTTVDLKMTSLTTEDTATYFCARGNVGSTGVSIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 125 - protein, 438 aa, Artificial Sequence
  synthetic polypeptide
QSVEESGGRLVAPGTPLTLTCTVSGFSLSYFGMYWVRQAPGRGLEWIGTIDSSDIIYYASWAKGRFTISKTSTTVDLKITSPTTEDTATYFCARSGGVAGGDSVWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 126 - protein, 436 aa, Artificial Sequence
  synthetic polypeptide
QSLEESGGRLVTPGGYLTLTCTVSGFSLSSYDMGWVRQAPGKGLEWIGYIFINDNTYYATWAKGRFTISKTSTTMDLKMTSLTTEDTATYFCVRGYFGGMDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 127 - protein, 437 aa, Artificial Sequence
  synthetic polypeptide
QSVEESGGRLVTPGASLTLTCTVSGIDFSTNAMTWVRQAPGKGLEWIGYIYEGSGNTFYASWAKGRFTISRTSTTVDLKMTSLTMEDTATYFCARGYLGAMDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 128 - protein, 437 aa, Artificial Sequence
  synthetic polypeptide
QSVEESGGGLVKPGASLTLTCQVSGFSLSDHYMSWVRQAPGKGLEWVAYISEGGATYYASWAKGRFTISKTSSTTVDLKMTSLTTEDTATYFCARGWLAAFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 129 - DNA, 336 bp, Artificial Sequence
  synthetic polynucleotide
gcgcttgtgatgacccagactccatcccctgtgtctgcagctgtgggaggcacagtcaccatcaactgccaggccagtcagagtgttcttaataacaactacttatcctggtatcagcagaaaccagggcagcctcccaagctcctgatctattctgcatccaaactggcaactggggtcccatcgcggttcagcggcagtggatctgggacacagtttactctcaccatcagcggcgtgcagtgtgacgatgctgccacttactactgtgcaggctataaaaatttcggtaatgatgataatgctttcggcggagggaccgaggtggtggtcaaa

SEQ ID NO: 130 - DNA, 333 bp, Artificial Sequence
  synthetic polynucleotide
gcgcaagtgctgacccagacatcatcccccgtgtctgtaaatatgggaggcacagtcaccatcaactgccagtccagtctgagtgttaataggagcgacttatcctggtatcagcagaaaccagggcagcctcccaaactcctgatctatctggcatccaatctggaatctggggtcccctcgcggttcaaaggcagtggatctgggacgcacttcactctcaccatcaacggcgtggaatgtgacgatgctgccacttactactgtgcaggcggttatagtagtggtaatgataggaatgctttcggcggagggaccgaggtggtggtcaaa

SEQ ID NO: 131 - DNA, 336 bp, Artificial Sequence
  synthetic polynucleotide
gcgcttgtgatgacccagactccatcccctgtgtctgcagctgtgggaggcacagtcaccatcagttgccaggccagtcagagtgttgctaataacaactacttagcctggtatcagcagaaaccagggcagcctcccaagctcctgatttaccaggcatccactctggcatctggggtcccatcgcggttcaaaggcagtggatctgggacacagttcactctcaccatcagtggcgtggagtgtgacgatgctgccacttactactgtgcaggctatcgaagttatattaatgctgataatgctttcggcggagggaccgaggtggtggtcgaa

SEQ ID NO: 132 - DNA, 336 bp, Artificial Sequence
  synthetic polynucleotide
gcccaagtgctgacccagactccatcctccgtgtctgcagctgtgggaggcacagtcaccatcaattgccagtccagtcagagtgtttggcagaaaaactggttatcctggtttcagcagaaaccagggcagcctcccaagctcctgatctacagggcgtccactctggcatctggggtcccatcgcggttcaagagcagtggatctgggacacagttcactctcaccatcagcggcgtgcagtgtgacgatgctgccacttactactgtgcaggcgcttatagtagtaatagtgatgttagggctttcggcggagggaccgagctggtggtcaaa

SEQ ID NO: 133 - DNA, 339 bp, Artificial Sequence
  synthetic polynucleotide
gcccaagtgctgacccagactgcatcgcccgtgtctgcagctgtgggaagcacagtcaccatcaattgccaggccagtcagagtgtttataataacaacaacttagcctggtatcagcagaaaccagggcagcctcccaagctcctgatctattatacatccactctggcatctggggtctcatcgcggttcaaaggcagtggatctgggacacagttcactctcaccatcagcggcatgcagtgtgacgatgctgccacttactactgtcaaggcgaatttgcttgtagtactgctgattgtcttactttcggcggagggaccgaggtggtggtcaaa

SEQ ID NO: 134 - DNA, 330 bp, Artificial Sequence
  synthetic polynucleotide
gcgcttgtgatgacccagactccagcctccgtgtctgcagctgtgggaggcacagtcaccatcaattgccaggccagtgagagcattagcagttggttagcctggtatcagcagaaaccagggcagcctcccaaactcctgatctattatgcatccactctggcatctggggtctcatcgcggttcaaaggcagtggatctaggacagagtacactcttaccatcagcgacctggagtgtgccgatgctgccacttactactgtgcaggctatggaagtgctaatgatgataaaaatggtttcggcggagggaccgaggtggtggtcaaa

SEQ ID NO: 135 - DNA, 330 bp, Artificial Sequence
  synthetic polynucleotide
gcctatgatatgacccagactccagcctctgtggaggtagctgtgggaggcacagtcaccatcaagtgccaggccagtcagagtattaacaatgaattggcctggtatcagcagaaaccagggcagcctcccaagctcctgatctatctggcattcactctggcatctggggtcccatcgcggttcaaaggcagtagatctgggacagagttcactctcaccattagcgacctggagtgtgccgatgctgccacttactactgtcaacagggttatattattagtggtgttgataatgttttcggcggagggaccgaggtggtggtcaaa

SEQ ID NO: 136 - DNA, 336 bp, Artificial Sequence
  synthetic polynucleotide
gcccaagtgctgacccagactccatcctccgtgtctgcagctgtgggaggcacagtcaccatcaattgccagtccagtcagagtgtttgggagaaaaactggttatcctggtttcagcagaaaccagggcagcctcccaagctcctgatctacagggcatccactctggcatctggggtcccatcgcggttcaagagcagtggatctgggacacagttcactctcaccatcagcggcgtgcagtgtgacgatgctgccgtttattattgtgcaggcgcttatagtgttaatagtgatgttagggctttcggcggagggaccgagctggtggtcaaa

SEQ ID NO: 137 - DNA, 336 bp, Artificial Sequence
  synthetic polynucleotide
gccatcgtgatgacccagactccatcttccaagtctgtccctgtgggaggcacagtcaccatcaattgccagtccagtgagagtgtttatgagaacaacgacttatcctggtatcagcagaaaccagggcagcctcccaagctcctgatctactgggcatccagcctggcatctggggtcccatcgcggttcgaaggcagtggatctgggacacagttcactctcaccatcagcaatgtggtgtgtgacgatgctgccacttactactgtgcaggatataaaagtatgagtactgatggctttgctttcggcggagggaccgaggtggtggtcaaa

SEQ ID NO: 138 - DNA, 336 bp, Artificial Sequence
  synthetic polynucleotide
gccatcgtgatgacccagactccatcttccaagtctgtccctgtgggaggcacagtcaccatcagttgccaggccagtcagagtctttataagaacaccgacttagcctggtttcaacagaaaccagggcagcctcccaaactcctgatctattttgcatcgaatctggcatctggagtcacatcgcggttcaaaggcagtggatctgggacacagttcactctcaccatcagcgatgtggtgtgtgacgatgctgccacttactactgtgcaggatataaaagtagtactactgatgggtttggtttcggcggagggaccgaggtggtggtcaaa

SEQ ID NO: 139 - DNA, 330 bp, Artificial Sequence
  synthetic polynucleotide
gcgcaagtgctggcccagactccatcctccgtgtctgcagctgtgggaggcacagtcaccatcgattgccagtccagtcagagtgttggtaataacgcctatttatcctggtatcagcagaaaccagggcagcctcccaagctcctgatctattatgcatccactctggcatctggggtcccctcccggttcagtggcagtggatctgggacacacttcactctcaccatcagcggcgtgcagtgtgacgatgctgccacttactactgtcaagcctattaccatgttggtgttgctgctttcggcggagggaccgaggtggtggtcaaa

SEQ ID NO: 140 - DNA, 330 bp, Artificial Sequence
  synthetic polynucleotide
gacgtcgtgatgacccagactccagcctccgtgtctgaatctgtgggaggcacagtcaccatcaagtgccaggccagtcaggatctttatagtaattgtttatcctggtatcagcagaaaccagggcagcgtcccaagctcctgatgtatctgacatccactctggcatctggggtcccatcgcggttcaaaggcagtggatctgggacagatttcactctcaccatcagcgacctggagtgtgccgatgctgccacttactactgtcaaggcttccatggttatggtgttggtgccgctttcggcggagggaccgaggtggtggtcaag

SEQ ID NO: 141 - DNA, 330 bp, Artificial Sequence
  synthetic polynucleotide
gacgtcgtgatgacccagactccagcctccgtggaggcagctgtgggaggcacagtcaccatcaagtgccaggccagtcagagcattaatagttggttatcctggtatcagcagaaaccagggcagcgtcccaaactcctgatctatgctgcatccactctggcatctggggtctcatcgcggttcaaaggcagtaaatctgggacagagttcactctcaccatcagcggtgtgcagtgtgacgatgctgccacttattattgtcaaggctatgatggtagtagtggtagtgctgctagtttcggcggagggaccgaggtggtggtcaaa

SEQ ID NO: 142 - DNA, 363 bp, Artificial Sequence
  synthetic polynucleotide
cagtcgctggaggagtccgggggagacctggtcaagcctggggcatccctgacactcacctgcacagcctctggattctccttcagtagcagctattacatgtggtgggtccgccaggctccagggaaggggctggagtggatcgcatgcattaatggtggtagtcgtggtaccacttactacgcgagctgggcgaaaggccgattcaccatctccaaaacctcgtcgaccacggtgactctgcaaatgaccagtctgacagtcgcggacacggccacctatttctgtgcgagagatccatatggttttagtggtagtatttatgccttgtggggcccaggcaccctggtcaccgtctcctca

SEQ ID NO: 143 - DNA, 348 bp, Artificial Sequence
  synthetic polynucleotide
cagtcgctggaggagtccgggggtcgcctggtcacgcctgggacacccctgacactctcctgcaaagcctctggactctccctcagtggccactacatgagctgggtccgccaggctccagggaaggggctggagtggatcggacacatttatggtagtggccgtggtttatggtacgcgaactgggcgaaaggccgattcaccatctccaagacctcgaccacggtagatctgaaaattatcagtccgacaaacgaggacacggccacctatttctgtgccagatatcgccctgttgattatgtgatggacatctggggcccaggcaccctggtcaccgtctcgtta

SEQ ID NO: 144 - DNA, 366 bp, Artificial Sequence
  synthetic polynucleotide
caggagcagctggaggagtccgggggagacctggtcaagcctgagggatccctgacactcacctgcacagcctctggattctccttcagtagcaactactacatgtgctgggtccgccaggctccagggaagggactggagtggatcgcatgcatttatgctggtagtagtggtagcacttactacgcgagctgggcgaaaggccggttcaccgtctccaaaacctcgtcgaccacggtgactctgcaaatgaccagtctgacagccgcggacacggccacctatttctgtgcgagagagcgtgaaaattatattggtgttggttactatttgtggggcccaggcaccctggtcaccgtctcctca

SEQ ID NO: 145 - DNA, 348 bp, Artificial Sequence
  synthetic polynucleotide
gagcagctgaaggagtccgggggtcgcctggtcacgcctgggacacccctgacactcacctgcaccgtctctggcttctccctcagtggcagctattggataaattgggttcgccaggctccagggaaggggctggaatggatcgcgattattgcggcaggtgggcgtatatggtacgcgagctcggtgaaaggccgattcaccatctccaaaacctcgaccacggtggatctgaaaatgaccagtccgacaaccgaggacacggccacctatttctgtgccagagacgatattggtattcctggtggggacatctggggcccaggcaccctggtcaccgtctcctta

SEQ ID NO: 146 - DNA, 363 bp, Artificial Sequence
  synthetic polynucleotide
caggagcagctgaaggagtccggaggaggcctggtaacgcctggaggaaccctgacactcacctgcacagcctctggattcaccatcaatagttacttcatgagctgggtccgccaggctccagggaagggactggaatggatcggaatcatttatggtcgtgataagacatactacgcgacctggacgaaaggccgattcaccatctccaaaacctcgaccacggtggatctgataatcaccagtccgacaaccgaggacacggccacctatttctgtgccagaagttctagtagtggtaggggtttatattacggtggcatggacccctggggcccagggaccctcgtcaccgtctcttca

SEQ ID NO: 147 - DNA, 366 bp, Artificial Sequence
  synthetic polynucleotide
cagtcgttggaggagtccgggggagacctggtcaagcctggggcatccctgacactcacctgcacagcctctggattctccttcagtagcggctactacatgtgctgggtccgccaggctccagggaaggggctggagtggatcgcatgtatttatgctggtagtggtgctagaacttactacgcgagctgggcgaaaggccgattcaccatctccaaaacctcgtcgaccacggtgactctgcaaatgaccagtctgacagccgcggacacggccacctatttctgtgcgagagacccatatagcgtgaatgatcctgttggcgataccttgtggggcccaggcaccctggtcaccgtctcctca

SEQ ID NO: 148 - DNA, 363 bp, Artificial Sequence
  synthetic polynucleotide
gagcgcctggaggagtccgggggagacctggtcaagcctggggcatccctgacactcacctgcaaagcctctggagccgacttcaataacaactacataatgtgttgggtccgccaggctccagggaaggggctggagtggatcgcatgcattagcactctcactactgccacttactacgcgacctgggcaaaaggccgattcaccatctccacaacctcgtcgaccacggtgactctgcaaatgaccagcctgacagccgcggacacggccacctatttctgtgcgggagatggttggagtggtgatggtgtgatcacatttaacttgtggggcccaggcaccctggtcaccgtctcctca

SEQ ID NO: 149 - DNA, 348 bp, Artificial Sequence
  synthetic polynucleotide
cagtcgctggaggagtccgggggtcgcctggtcacgcctgggacacccttgacactcacctgcacagcctctggattctccctcagtagcagctactggataaactgggttcgccaggctccagggaagggcctggaatggatcgcgattatgccggcaggtgggcgtccttactacgcgacctgggcaaaaggccgattcatcatctccaaaacctcgaccacggtggatctgaaaatgaccagtccgacaaccgaggacacggccacctatttctgtgccagagacgatattggtactcctggtggggacatctggggcccaggcaccctggtcaccgtctcctta

SEQ ID NO: 150 - DNA, 342 bp, Artificial Sequence
  synthetic polynucleotide
cagtcggtggaggagtccgggggtcgcctggtcacgcctgggacacccctgacactcacctgcacagtctctagaatcgacctcagtagatatgtggtggactgggtccgccaggctccaggggaggggctggaatggatcggaaccattggttatggtagcacatggtacgcgagctgggtgaaaggccgattcaccatctccagaacctcgaccacggtggatctgaaaatgaccagtctgacaaccgaggacacggccacctatttctgtgccaggggaaatgttgggagtactggggtcagcatctggggcccaggcacgctggtcaccgtctcctta

SEQ ID NO: 151 - DNA, 345 bp, Artificial Sequence
  synthetic polynucleotide
cagtcggtggaggagtccgggggtcgcctggtcgcgcctgggacacccctgacactcacctgcaccgtctctggattctccctcagttactttggaatgtactgggtccgccaggctccagggagggggctggaatggatcggaaccattgatagtagtgatatcatatactacgcgagctgggcgaaaggccgattcaccatctccaaaacctcgaccacggtggatctgaaaatcaccagtccgacaaccgaggacacggccacctatttctgtgccagatctggtggtgtggctggtggagatagcgtctggggcccaggcaccctggtcaccgtctcctta

SEQ ID NO: 152 - DNA, 339 bp, Artificial Sequence
  synthetic polynucleotide
cagtcactggaggagtccgggggtcgcctggtcacgcctggaggatacctgacactcacctgcacagtctctggattctccctcagcagctatgacatgggctgggtccgccaggctccagggaaggggctggagtggatcggatacatttttattaatgataacacatactacgcgacctgggcgaaaggccgattcaccatctccaagacctcgaccacgatggatctgaaaatgaccagtctgacaaccgaggacacggccacctatttctgtgtcagaggatattttggcggcatggacccctggggcccagggaccctcgtcaccgtctcttca

SEQ ID NO: 153 - DNA, 342 bp, Artificial Sequence
  synthetic polynucleotide
cagtcggtggaggagtccgggggtcgcctggtcacgcctggggcatccctgacactcacctgcacagtctctggaatcgacttcagtaccaatgcaatgacctgggtccgccaggctccagggaaggggctggaatggatcggatatatttatgagggtagtggtaatacattctacgcgagctgggcgaaaggccgattcaccatttccagaacctcgaccacggtggatttgaaaatgaccagtctgacaatggaggacacggccacctatttctgtgccagaggatatctcggcgccatggacccctggggcccagggaccctcgtcaccgtctcttca

SEQ ID NO: 154 - DNA, 342 bp, Artificial Sequence
  synthetic polynucleotide
cagtcagtggaggagtccgggggaggcctggtcaagcctggggcatccctgacactcacctgtcaagtctctggattctccctcagtgaccactacatgagctgggtccgccaggctccagggaaggggctggaatgggtcgcatatattagtgagggtggtgccacatactacgcgagctgggcaaaaggccgattcaccatctccaaaacctcgtcgaccacggtggatctgaaaatgaccagtctgacaaccgaggacacggccacctatttttgtgccagaggatggcttgctgcttttgatccctggggcccaggcaccctggtcaccgtctcctca

SEQ ID NO: 155 - DNA, 648 bp, Artificial Sequence
  synthetic polynucleotide
gcgcttgtgatgacccagactccatcccctgtgtctgcagctgtgggaggcacagtcaccatcaactgccaggccagtcagagtgttcttaataacaactacttatcctggtatcagcagaaaccagggcagcctcccaagctcctgatctattctgcatccaaactggcaactggggtcccatcgcggttcagcggcagtggatctgggacacagtttactctcaccatcagcggcgtgcagtgtgacgatgctgccacttactactgtgcaggctataaaaatttcggtaatgatgataatgctttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 156 - DNA, 645 bp, Artificial Sequence
  synthetic polynucleotide
gcgcaagtgctgacccagacatcatcccccgtgtctgtaaatatgggaggcacagtcaccatcaactgccagtccagtctgagtgttaataggagcgacttatcctggtatcagcagaaaccagggcagcctcccaaactcctgatctatctggcatccaatctggaatctggggtcccctcgcggttcaaaggcagtggatctgggacgcacttcactctcaccatcaacggcgtggaatgtgacgatgctgccacttactactgtgcaggcggttatagtagtggtaatgataggaatgctttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 157 - DNA, 648 bp, Artificial Sequence
  synthetic polynucleotide
gcgcttgtgatgacccagactccatcccctgtgtctgcagctgtgggaggcacagtcaccatcagttgccaggccagtcagagtgttgctaataacaactacttagcctggtatcagcagaaaccagggcagcctcccaagctcctgatttaccaggcatccactctggcatctggggtcccatcgcggttcaaaggcagtggatctgggacacagttcactctcaccatcagtggcgtggagtgtgacgatgctgccacttactactgtgcaggctatcgaagttatattaatgctgataatgctttcggcggagggaccgaggtggtggtcgaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 158 - DNA, 648 bp, Artificial Sequence
  synthetic polynucleotide
gcccaagtgctgacccagactccatcctccgtgtctgcagctgtgggaggcacagtcaccatcaattgccagtccagtcagagtgtttggcagaaaaactggttatcctggtttcagcagaaaccagggcagcctcccaagctcctgatctacagggcgtccactctggcatctggggtcccatcgcggttcaagagcagtggatctgggacacagttcactctcaccatcagcggcgtgcagtgtgacgatgctgccacttactactgtgcaggcgcttatagtagtaatagtgatgttagggctttcggcggagggaccgagctggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 159 - DNA, 651 bp, Artificial Sequence
  synthetic polynucleotide
gcccaagtgctgacccagactgcatcgcccgtgtctgcagctgtgggaagcacagtcaccatcaattgccaggccagtcagagtgtttataataacaacaacttagcctggtatcagcagaaaccagggcagcctcccaagctcctgatctattatacatccactctggcatctggggtctcatcgcggttcaaaggcagtggatctgggacacagttcactctcaccatcagcggcatgcagtgtgacgatgctgccacttactactgtcaaggcgaatttgcttgtagtactgctgattgtcttactttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 160 - DNA, 642 bp, Artificial Sequence
  synthetic polynucleotide
gcgcttgtgatgacccagactccagcctccgtgtctgcagctgtgggaggcacagtcaccatcaattgccaggccagtgagagcattagcagttggttagcctggtatcagcagaaaccagggcagcctcccaaactcctgatctattatgcatccactctggcatctggggtctcatcgcggttcaaaggcagtggatctaggacagagtacactcttaccatcagcgacctggagtgtgccgatgctgccacttactactgtgcaggctatggaagtgctaatgatgataaaaatggtttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 161 - DNA, 642 bp, Artificial Sequence
  synthetic polynucleotide
gcctatgatatgacccagactccagcctctgtggaggtagctgtgggaggcacagtcaccatcaagtgccaggccagtcagagtattaacaatgaattggcctggtatcagcagaaaccagggcagcctcccaagctcctgatctatctggcattcactctggcatctggggtcccatcgcggttcaaaggcagtagatctgggacagagttcactctcaccattagcgacctggagtgtgccgatgctgccacttactactgtcaacagggttatattattagtggtgttgataatgttttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 162 - DNA, 648 bp, Artificial Sequence
  synthetic polynucleotide
gcccaagtgctgacccagactccatcctccgtgtctgcagctgtgggaggcacagtcaccatcaattgccagtccagtcagagtgtttgggagaaaaactggttatcctggtttcagcagaaaccagggcagcctcccaagctcctgatctacagggcatccactctggcatctggggtcccatcgcggttcaagagcagtggatctgggacacagttcactctcaccatcagcggcgtgcagtgtgacgatgctgccgtttattattgtgcaggcgcttatagtgttaatagtgatgttagggctttcggcggagggaccgagctggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 163 - DNA, 648 bp, Artificial Sequence
  synthetic polynucleotide
gccatcgtgatgacccagactccatcttccaagtctgtccctgtgggaggcacagtcaccatcaattgccagtccagtgagagtgtttatgagaacaacgacttatcctggtatcagcagaaaccagggcagcctcccaagctcctgatctactgggcatccagcctggcatctggggtcccatcgcggttcgaaggcagtggatctgggacacagttcactctcaccatcagcaatgtggtgtgtgacgatgctgccacttactactgtgcaggatataaaagtatgagtactgatggctttgctttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 164 - DNA, 648 bp, Artificial Sequence
  synthetic polynucleotide
gccatcgtgatgacccagactccatcttccaagtctgtccctgtgggaggcacagtcaccatcagttgccaggccagtcagagtctttataagaacaccgacttagcctggtttcaacagaaaccagggcagcctcccaaactcctgatctattttgcatcgaatctggcatctggagtcacatcgcggttcaaaggcagtggatctgggacacagttcactctcaccatcagcgatgtggtgtgtgacgatgctgccacttactactgtgcaggatataaaagtagtactactgatgggtttggtttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 165 - DNA, 642 bp, Artificial Sequence
  synthetic polynucleotide
gcgcaagtgctggcccagactccatcctccgtgtctgcagctgtgggaggcacagtcaccatcgattgccagtccagtcagagtgttggtaataacgcctatttatcctggtatcagcagaaaccagggcagcctcccaagctcctgatctattatgcatccactctggcatctggggtcccctcccggttcagtggcagtggatctgggacacacttcactctcaccatcagcggcgtgcagtgtgacgatgctgccacttactactgtcaagcctattaccatgttggtgttgctgctttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 166 - DNA, 642 bp, Artificial Sequence
  synthetic polynucleotide
gacgtcgtgatgacccagactccagcctccgtgtctgaatctgtgggaggcacagtcaccatcaagtgccaggccagtcaggatctttatagtaattgtttatcctggtatcagcagaaaccagggcagcgtcccaagctcctgatgtatctgacatccactctggcatctggggtcccatcgcggttcaaaggcagtggatctgggacagatttcactctcaccatcagcgacctggagtgtgccgatgctgccacttactactgtcaaggcttccatggttatggtgttggtgccgctttcggcggagggaccgaggtggtggtcaagggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 167 - DNA, 642 bp, Artificial Sequence
  synthetic polynucleotide
gacgtcgtgatgacccagactccagcctccgtggaggcagctgtgggaggcacagtcaccatcaagtgccaggccagtcagagcattaatagttggttatcctggtatcagcagaaaccagggcagcgtcccaaactcctgatctatgctgcatccactctggcatctggggtctcatcgcggttcaaaggcagtaaatctgggacagagttcactctcaccatcagcggtgtgcagtgtgacgatgctgccacttattattgtcaaggctatgatggtagtagtggtagtgctgctagtttcggcggagggaccgaggtggtggtcaaaggtgatccagttgcacctactgtcctcctcttcccaccatctagcgatgaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcagtaggaagaactgt

SEQ ID NO: 168 - DNA, 1332 bp, Artificial Sequence
  synthetic polynucleotide
cagtcgctggaggagtccgggggagacctggtcaagcctggggcatccctgacactcacctgcacagcctctggattctccttcagtagcagctattacatgtggtgggtccgccaggctccagggaaggggctggagtggatcgcatgcattaatggtggtagtcgtggtaccacttactacgcgagctgggcgaaaggccgattcaccatctccaaaacctcgtcgaccacggtgactctgcaaatgaccagtctgacagtcgcggacacggccacctatttctgtgcgagagatccatatggttttagtggtagtatttatgccttgtggggcccaggcaccctggtcaccgtctcctcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 169 - DNA, 1317 bp, Artificial Sequence
  synthetic polynucleotide
cagtcgctggaggagtccgggggtcgcctggtcacgcctgggacacccctgacactctcctgcaaagcctctggactctccctcagtggccactacatgagctgggtccgccaggctccagggaaggggctggagtggatcggacacatttatggtagtggccgtggtttatggtacgcgaactgggcgaaaggccgattcaccatctccaagacctcgaccacggtagatctgaaaattatcagtccgacaaacgaggacacggccacctatttctgtgccagatatcgccctgttgattatgtgatggacatctggggcccaggcaccctggtcaccgtctcgttagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 170 - DNA, 1335 bp, Artificial Sequence
  synthetic polynucleotide
caggagcagctggaggagtccgggggagacctggtcaagcctgagggatccctgacactcacctgcacagcctctggattctccttcagtagcaactactacatgtgctgggtccgccaggctccagggaagggactggagtggatcgcatgcatttatgctggtagtagtggtagcacttactacgcgagctgggcgaaaggccggttcaccgtctccaaaacctcgtcgaccacggtgactctgcaaatgaccagtctgacagccgcggacacggccacctatttctgtgcgagagagcgtgaaaattatattggtgttggttactatttgtggggcccaggcaccctggtcaccgtctcctcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 171 - DNA, 1317 bp, Artificial Sequence
  synthetic polynucleotide
gagcagctgaaggagtccgggggtcgcctggtcacgcctgggacacccctgacactcacctgcaccgtctctggcttctccctcagtggcagctattggataaattgggttcgccaggctccagggaaggggctggaatggatcgcgattattgcggcaggtgggcgtatatggtacgcgagctcggtgaaaggccgattcaccatctccaaaacctcgaccacggtggatctgaaaatgaccagtccgacaaccgaggacacggccacctatttctgtgccagagacgatattggtattcctggtggggacatctggggcccaggcaccctggtcaccgtctccttagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 172 - DNA, 1332 bp, Artificial Sequence
  synthetic polynucleotide
caggagcagctgaaggagtccggaggaggcctggtaacgcctggaggaaccctgacactcacctgcacagcctctggattcaccatcaatagttacttcatgagctgggtccgccaggctccagggaagggactggaatggatcggaatcatttatggtcgtgataagacatactacgcgacctggacgaaaggccgattcaccatctccaaaacctcgaccacggtggatctgataatcaccagtccgacaaccgaggacacggccacctatttctgtgccagaagttctagtagtggtaggggtttatattacggtggcatggacccctggggcccagggaccctcgtcaccgtctcttcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 173 - DNA, 1335 bp, Artificial Sequence
  synthetic polynucleotide
cagtcgttggaggagtccgggggagacctggtcaagcctggggcatccctgacactcacctgcacagcctctggattctccttcagtagcggctactacatgtgctgggtccgccaggctccagggaaggggctggagtggatcgcatgtatttatgctggtagtggtgctagaacttactacgcgagctgggcgaaaggccgattcaccatctccaaaacctcgtcgaccacggtgactctgcaaatgaccagtctgacagccgcggacacggccacctatttctgtgcgagagacccatatagcgtgaatgatcctgttggcgataccttgtggggcccaggcaccctggtcaccgtctcctcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 174 - DNA, 1332 bp, Artificial Sequence
  synthetic polynucleotide
gagcgcctggaggagtccgggggagacctggtcaagcctggggcatccctgacactcacctgcaaagcctctggagccgacttcaataacaactacataatgtgttgggtccgccaggctccagggaaggggctggagtggatcgcatgcattagcactctcactactgccacttactacgcgacctgggcaaaaggccgattcaccatctccacaacctcgtcgaccacggtgactctgcaaatgaccagcctgacagccgcggacacggccacctatttctgtgcgggagatggttggagtggtgatggtgtgatcacatttaacttgtggggcccaggcaccctggtcaccgtctcctcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 175 - DNA, 1317 bp, Artificial Sequence
  synthetic polynucleotide
cagtcgctggaggagtccgggggtcgcctggtcacgcctgggacacccttgacactcacctgcacagcctctggattctccctcagtagcagctactggataaactgggttcgccaggctccagggaagggcctggaatggatcgcgattatgccggcaggtgggcgtccttactacgcgacctgggcaaaaggccgattcatcatctccaaaacctcgaccacggtggatctgaaaatgaccagtccgacaaccgaggacacggccacctatttctgtgccagagacgatattggtactcctggtggggacatctggggcccaggcaccctggtcaccgtctccttagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 176 - DNA, 1311 bp, Artificial Sequence
  synthetic polynucleotide
cagtcggtggaggagtccgggggtcgcctggtcacgcctgggacacccctgacactcacctgcacagtctctagaatcgacctcagtagatatgtggtggactgggtccgccaggctccaggggaggggctggaatggatcggaaccattggttatggtagcacatggtacgcgagctgggtgaaaggccgattcaccatctccagaacctcgaccacggtggatctgaaaatgaccagtctgacaaccgaggacacggccacctatttctgtgccaggggaaatgttgggagtactggggtcagcatctggggcccaggcacgctggtcaccgtctccttagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 177 - DNA, 1314 bp, Artificial Sequence
  synthetic polynucleotide
cagtcggtggaggagtccgggggtcgcctggtcgcgcctgggacacccctgacactcacctgcaccgtctctggattctccctcagttactttggaatgtactgggtccgccaggctccagggagggggctggaatggatcggaaccattgatagtagtgatatcatatactacgcgagctgggcgaaaggccgattcaccatctccaaaacctcgaccacggtggatctgaaaatcaccagtccgacaaccgaggacacggccacctatttctgtgccagatctggtggtgtggctggtggagatagcgtctggggcccaggcaccctggtcaccgtctccttagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 178 - DNA, 1308 bp, Artificial Sequence
  synthetic polynucleotide
cagtcactggaggagtccgggggtcgcctggtcacgcctggaggatacctgacactcacctgcacagtctctggattctccctcagcagctatgacatgggctgggtccgccaggctccagggaaggggctggagtggatcggatacatttttattaatgataacacatactacgcgacctgggcgaaaggccgattcaccatctccaagacctcgaccacgatggatctgaaaatgaccagtctgacaaccgaggacacggccacctatttctgtgtcagaggatattttggcggcatggacccctggggcccagggaccctcgtcaccgtctcttcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 179 - DNA, 1311 bp, Artificial Sequence
  synthetic polynucleotide
cagtcggtggaggagtccgggggtcgcctggtcacgcctggggcatccctgacactcacctgcacagtctctggaatcgacttcagtaccaatgcaatgacctgggtccgccaggctccagggaaggggctggaatggatcggatatatttatgagggtagtggtaatacattctacgcgagctgggcgaaaggccgattcaccatttccagaacctcgaccacggtggatttgaaaatgaccagtctgacaatggaggacacggccacctatttctgtgccagaggatatctcggcgccatggacccctggggcccagggaccctcgtcaccgtctcttcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 180 - DNA, 1311 bp, Artificial Sequence
  synthetic polynucleotide
cagtcagtggaggagtccgggggaggcctggtcaagcctggggcatccctgacactcacctgtcaagtctctggattctccctcagtgaccactacatgagctgggtccgccaggctccagggaaggggctggaatgggtcgcatatattagtgagggtggtgccacatactacgcgagctgggcaaaaggccgattcaccatctccaaaacctcgtcgaccacggtggatctgaaaatgaccagtctgacaaccgaggacacggccacctatttttgtgccagaggatggcttgctgcttttgatccctggggcccaggcaccctggtcaccgtctcctcagggcaacctaaggctccatcagtcttcccactggccccctgctgcggggacacacccagctccacggtgaccttgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 181 - RNA, 20 nt, Artificial Sequence
  synthetic modified polynucleotide
  modified_base (1)..(20): combination of 2′-fluoro, 2′-O-methyl, inverted deoxyadenosine, phosphorothioate linked ribonucleotides
cagccccuuauuguuauacg

SEQ ID NO: 182 - RNA, 20 nt, Artificial Sequence
  synthetic modified polynucleotide
  modified_base (1)..(20): combination of 2′-fluoro, 2′-O-methyl, inverted deoxyadenosine, phosphorothioate linked ribonucleotides
uucuuucucaagaacgcugc

SEQ ID NO: 183 - RNA, 21 nt, Artificial Sequence
  synthetic modified polynucleotide
  modified_base (1)..(21): combination of 2′-fluoro, 2′-O-methyl, inverted abasic, phosphorothioate linked ribonucleotides
gccccuuauuguuauacgauu

SEQ ID NO: 184 - RNA, 21 nt, Artificial Sequence
  synthetic modified polynucleotide
  modified_base (1)..(21): combination of 2′-fluoro, 2′-O-methyl, phosphorothioate linked ribonucleotides
gugggaagaaagaugaaguuu

SEQ ID NO: 185 - RNA, 20 nt, Artificial Sequence
  synthetic modified polynucleotide
  modified_base (1)..(20): combination of 2′-fluoro, 2′-O-methyl, inverted abasic, phosphorothioate linked ribonucleotides
gaggacugugcccacuucac

SEQ ID NO: 186 - RNA, 20 nt, Artificial Sequence
  synthetic modified polynucleotide
  modified_base (1)..(20): combination of 2′-fluoro, 2′-O-methyl, inverted abasic, phosphorothioate linked ribonucleotides
augugggaagaaagaugaag

SEQ ID NO: 187 - RNA, 20 nt, Artificial Sequence
  synthetic modified polynucleotide
  modified_base (1)..(20): combination of 2′-fluoro, 2′-O-methyl, inverted abasic, phosphorothioate linked ribonucleotides
cccuaucaugaccaaggagu

SEQ ID NO: 188 - RNA, 20 nt, Artificial Sequence
  synthetic modified polynucleotide
  modified_base (1)..(20): combination of 2′-fluoro, 2′-O-methyl, inverted abasic, phosphorothioate linked ribonucleotides
gggaagaaagaugaagucgc

SEQ ID NO: 189 - RNA, 21 nt, Artificial Sequence
  synthetic modified polynucleotide
  modified_base (1)..(21): combination of 2′-fluoro, 2′-O-methyl, inverted abasic, phosphorothioate linked ribonucleotides
augccuuucuacaguggcuuu

SEQ ID NO: 190 - RNA, 20 nt, Artificial Sequence
  synthetic modified polynucleotide
  modified_base (1)..(20): combination of 2′-fluoro, 2′-O-methyl, inverted abasic, phosphorothioate linked ribonucleotides
uccuaugacuguagauuuua

SEQ ID NO: 191 - RNA, 20 nt, Artificial Sequence
  synthetic modified polynucleotide
  modified_base (1)..(20): combination of 2′-fluoro, 2′-O-methyl, inverted abasic, phosphorothioate linked ribonucleotides
acacaaugcucagacgcaau

SEQ ID NO: 192 - RNA, 21 nt, Artificial Sequence
  synthetic modified polynucleotide
  modified_base (1)..(21): combination of 2′-fluoro, 2′-O-methyl, phosphorothioate linked ribonucleotides
ucguauaacaauaaggggcug

SEQ ID NO: 193 - RNA, 21 nt, Artificial Sequence
  synthetic modified polynucleotide
  modified_base (1)..(21): combination of 2′-fluoro, 2′-O-methyl, phosphorothioate linked ribonucleotides
ugcagcguucuugagaaagaa

SEQ ID NO: 194 - RNA, 21 nt, Artificial Sequence
  synthetic modified polynucleotide
  modified_base (1)..(21): combination of 2′-fluoro, 2′-O-methyl, phosphorothioate linked ribonucleotides
ucguauaacaauaaggggcuu

SEQ ID NO: 195 - RNA, 21 nt, Artificial Sequence
  synthetic modified polynucleotide
  modified_base (1)..(21): combination of 2′-fluoro, 2′-O-methyl, phosphorothioate linked ribonucleotides
acuucaucuuucuucccacuu

SEQ ID NO: 196 - RNA, 23 nt, Artificial Sequence
  synthetic modified polynucleotide
  modified_base (1)..(23): combination of 2′-fluoro, 2′-O-methyl, phosphorothioate linked ribonucleotides
agugaagugggcacaguccucuu

SEQ ID NO: 197 - RNA, 23 nt, Artificial Sequence
  synthetic modified polynucleotide
  modified_base (1)..(23): combination of 2′-fluoro, 2′-O-methyl, phosphorothioate linked ribonucleotides
acuucaucuuucuucccacauuu

SEQ ID NO: 198 - RNA, 23 nt, Artificial Sequence
  synthetic modified polynucleotide
  modified_base (1)..(23): combination of 2′-fluoro, 2′-O-methyl, phosphorothioate linked ribonucleotides
uacuccuuggucaugauaggguu

SEQ ID NO: 199 - RNA, 23 nt, Artificial Sequence
  synthetic modified polynucleotide
  modified_base (1)..(23): combination of 2′-fluoro, 2′-O-methyl, phosphorothioate linked ribonucleotides
agcgacuucaucuuucuucccuu

SEQ ID NO: 200 - RNA, 21 nt, Artificial Sequence
  synthetic modified polynucleotide
  modified_base (1)..(21): combination of 2′-fluoro, 2′-O-methyl, phosphorothioate linked ribonucleotides
agccacuguagaaaggcauuu

SEQ ID NO: 201 - RNA, 23 nt, Artificial Sequence
  synthetic modified polynucleotide
  modified_base (1)..(23): combination of 2′-fluoro, 2′-O-methyl, phosphorothioate linked ribonucleotides
auaaaaucuacagucauaggauu

SEQ ID NO: 202 - RNA, 21 nt, Artificial Sequence
  synthetic modified polynucleotide
  modified_base (1)..(21): combination of 2′-fluoro, 2′-O-methyl, phosphorothioate linked ribonucleotides
uugcgucugagcauuguguuu